(19) United States
(12) Patent Application Publication
(10) Pub. No.: US 2020/0261418 A1
(43) Pub. Date: Aug. 20, 2020

(54) HDAC INHIBITOR IN COMBINATION WITH IMMUNE CHECKPOINT MODULATOR FOR CANCER THERAPY

(71) Applicant: 4SC AG, Planegg-Martinsried (DE)

(72) Inventors: Susanne DANHAUSER-RIEDL, Baldham (DE); Frank HERMANN, Munich (DE); Roland BAUMGARTNER, Planegg (DE); Svetlana HAMM, Eichenau (DE); Rene BARTZ, Penzberg (DE)

(73) Assignee: 4SC AG, Planegg-Martinsried (DE)

(21) Appl. No.: 16/644,751

(22) PCT Filed: Sep. 7, 2018
(86) PCT No.: PCT/EP2018/074186

§ 371 Date: Mar. 5, 2020

(30) Foreign Application Priority Data
Sep. 8, 2017 (EP) 17190233.1
Sep. 8, 2017 (EP) 17190238.0
Sep. 8, 2017 (EP) 17190242.2

(51) Int. Cl. A61K 31/4155; A61P 35/00; A61K 39/395

(52) U.S. Cl. CPC A61K 31/4155; A61K 39/3955; A61P 35/00

(57) ABSTRACT

The invention relates to methods, compositions and uses for the of treatment of cancer comprising the administration of an HDAC inhibitor as defined herein for the treatment of cancer in combination with at least one immune checkpoint modulator as defined herein.

### FIELD OF APPLICATION OF THE INVENTION

The invention relates to medical applications of an HDAC inhibitor in combination with at least one immune checkpoint modulator in the treatment of cancer.

### KNOWN TECHNICAL BACKGROUND

Skin cancer is the most common human malignancy. Globally, there are about 2-3 million cases of skin cancer each year. The incidence of cutaneous malignant melanoma has been increasing worldwide, the annual increase is estimated between 3% to 7% (Giblin A V, Thomas J M. J Plast Reconstr Aesthet Surg 2007; 60: 32-40; Kasper B, et al. Crit Rev Oncol Hematol 2007; Markovic S N et al. Mayo Clin Proc 2007; 82: 364-380; Garbe C, Leiter U. Clin Dermatol 2009; 27: 3-9; Karim-Kos H E, et al. Eur J Cancer 2008; 44: 1345-1389), accounting to around 132,000 new cases of cutaneous melanoma per year internationally, as estimated by the WHO (WHO data found at: http://www.who.int/uv/faq/skincancer/en/indexl.html, accessed 2 Mar. 2010). The highest incidence rates have been reported for Australia, New Zealand, and the United States (US). The 2002 age-standardized (world) incidences ranged from 33.8 to 38.5/100,000 population for males and from 29.2 to 29.5/100,000 population for females for Australia and New Zealand, respectively (Giblin A V, Thomas J M. J Plast Reconstr Aesthet Surg 2007; 60: 32-40). Metastatic melanoma has been associated with a very poor prognosis as no widely effective therapy was available until recently. Treatment consisted mainly of single agent chemotherapy as dacarbazine or temozolomide. In the EU, dacarbazine is indicated as systemic therapy for the treatment of advanced melanoma regardless of line of therapy. Dacarbazine demonstrates an objective response rate (ORR) of 13% with a median OS ranging from 5.6 to 11 months among 8 randomized studies; and a 1-year OS rate ranging from 20% to 30% among 5 randomized studies (Yang A S, Chapman P B. Hematol Oncol Clin North Am. 2009; 3(3):583-97). In the past years, new treatment options in the targeted therapy (BRAF-(EMA. SmPC Tafinlar. (PDF-File), downloaded from http://ec.europa.eu/health/documents/community-register/2014/20140630128759/anx,_128759_en.pdf, accessed May 2017) and MEK-inhibitor (EMA. SmPC Mekinist. (PDF-File), downloaded from http://ec.europa.eu/health/documents/community-register/2014/20140630128759/anx_128759_en.pdf. Accessed May 2017) for BRAF-mutated melanoma) and the immuno-oncology area (regardless of BRAF-mutation status) were approved by regulatory agencies and substantially added to the therapeutic armamentarium for this disease. Recently, the role of the immune system in cancer has gained increasing interest and treatment options harnessing the body's own immune system have either already received marketing approval or are currently being developed. Of particular interest are treatments that abrogate the tumors' ability to effectively suppress immune responses by activating negative regulatory pathways (also called checkpoints) that are associated with immune homeostasis or allow the cancer to avoid destruction by the immune system over all. Two such checkpoints, cytotoxic T-lymphocyte protein 4 (CTLA4) and programmed cell death protein 1 (PD-1) and its ligand (PD-L1), have garnered the most attention so far. CTLA4 is a negative regulator of T cells that acts to control T-cell activation by competing with the co-stimulatory molecule CD28 for binding to shared ligands CD80 (also known as B7.1) and CD86 (also known as B7.2). The cell-surface receptor PD-1 is expressed by T cells on activation during priming or expansion and binds to one of two ligands, PD-L1 and PD-L2. Many types of cells can express PD-L1, including tumor cells and immune cells. Binding of PD-L1 or PD-L2 to PD-1 generates an inhibitory signal that attenuates the activity of T cells. Monoclonal antibodies that target either CTLA-4, PD-1 or PD-L1 can block this binding and boost the immune response against cancer cells.

In the field of immuno-oncology, the FDA granted approval for Ipilimumab “for the treatment of unresectable or metastatic melanoma” on 25 Mar. 2011. The EMA granted approval “for the treatment of advanced (unresectable or metastatic) melanoma in adults who have received prior therapy” on 13 Jul. 2011, and subsequently broadened the indication on 31 Oct. 2013 by granting approval for the first-line advanced melanoma. In 2015, the EMA approved with Nivolumab and Pembrolizumab two anti-PD1 antibodies for the treatment of advanced malignant melanoma. The approval for Pembrolizumab was mainly based on the results of the randomized Phase II study (Keynote-002) and a randomized Phase III (Keynote-006) study. The Keynote-002 study was an open-label, multi-center randomized Phase 2 trial to compare two dose levels of Pembrolizumab versus investigator-choice chemotherapy in patients with advanced melanoma. Patients were randomized to receive either Pembrolizumab 2 mg/kg every 3 weeks, Pembrolizumab 10 mg/kg every 3 weeks or investigator-choice chemotherapy.

Histone deacetylases (HDACs) are enzymes that catalyze the removal of acetyl groups from specific histone sites in particular at promoter and enhancer regions, which is an essential part of regulation of cellular gene transcription. HDACs also regulate gene expression in an indirect fashion by mediating the acetylation of non-histone proteins such as DNA-binding proteins, transcription factors, signal transducers, DNA repair and chaperon proteins (Ververis K et al., Biologics: Targets and Therapy 7: 47-60, 2013; Vitt D et al., Targeting histone acetylation. In: RSC Drug Discovery Series No. 48: Epigenetics for Drug Discovery. Editor: Nessa Carey. The Royal Society of Chemistry, 2016).

HDAC inhibitors have been described to cause growth arrest with subsequent differentiation or apoptosis of tumor cells, whereas normal cells are not affected. As summarized in a review article by Marks et al. (Nature Reviews Cancer, 2001, Volume 1, page 194-202), HDAC inhibitors cause cell-cycle arrest in G1 and/or G2 phase. Growth-inhibitory effects have been documented in vitro in virtually all transformed cell types, including cell lines that arise from both hematological and epithelial tumors. The growth inhibitory cellular mechanism of the HDAC inhibitors has been described as a specific induction of expression of the cell cycle inhibitor CDKN1A (p21). Additionally, this review article summarizes the induction of growth arrest in tumor-bearing mice by HDAC inhibitors. Efficacy of HDAC inhibitors has been demonstrated in animal models of diverse cancer types such as breast, prostate, lung and stomach cancers, neuroblastoma and leukemia.

Treatments of many cancer types by HDAC inhibitors have been described in the available literature. HDAC inhibition has an effect on the expression of a number of proteins playing pivotal roles in tumor-relevant processes, such as HER2/neu, VEGF, raf-1, cyclin A and B, Bax, Bad, p53, c-myc, Caspase 3, p21 and ERα. According to a review by Villar-Garea et al. (Int. J. Cancer: 112, 171-178 (2004)) cancer is understood to be an epigenetic as well as a genetic disease and the main goal using HDAC inhibitors would be restoration of gene expression of those tumor-suppressor genes that have been transcriptionally silenced by promoter-associated histone deacetylation. Drummond et al. (Annu. Rev. Pharmacol. Toxicol. 2005. 45:495-528) review the molecular mechanism and outcome of histone and non-histone substrates in cancer cells, which are effectors of HDAC, while HDAC also facilitates the acetylation of several key proteins other than histones. According to said review, acetylation is a key posttranslational modification of many proteins responsible for regulating critical intracellular pathways, and many of these substrates are tissue/development specific (EKLF, GATA-1, ERα, MyoD), oncogenic (c-Myb), tumor-suppressing (p53), or even rather ubiquitous (TFIIE, TFIIF, TCF, HNF-4) transcription factors. Modulation of those proteins can lead to induction of cell cycle arrest, differentiation and apoptosis, all of which are desirable mechanisms for treatment of cancer. Kelly et al. (Expert Opin Invest Drugs, 11(12), 2002) provides a further review on HDAC inhibitors in general and their application in cancer therapy.

The official US NIH website http://clinicaltrials.gov lists (status: February 2016) 545 clinical trials for cancer indications treated with HDAC inhibitors, among others various forms of leukemia (e.g. CML, CLL, AML), myelodysplastic syndrome, lymphoma including non-hodgkin's lymphoma, multiple myeloma, plasma cell neoplasm, solid tumors in general, small intestine cancer, mesothelioma, prostate, breast (male and female), lung cancer (including non-small and small cell), neuroendocrine, malignant epithelial neoplasms, pancreas, skin cancer (including melanoma), multiple myeloma, cervix, renal cell, head and neck, gastric, ovarian, liver cancer, colon, rectal, thymoma, fallopian tube, peritoneal, nasopharyngeal, vestibular schwannoma, meningioma, acoustic neuroma, neurofibromatosis type 2, thyroid, urothelial, gliomas, brain, esophagus, astrocytoma, anaplastic oligodendroglioma, giant cell glioblastoma, glioblastoma, gliosarcoma, mixed glioma and brain neoplasm.

4SC-202 (E)-N-(2-aminophenyl)-3-(1-((4-(1-methyl-1H-pyrazol-4-yl)phenyl)sulfonyl)-1H-pyrrol-3-yl)acrylamide is an orally available HDAC inhibitor histone-deacetylase (HDAC) inhibitor.

4SC-202 has been evaluated in a Phase I clinical trial (TOPAS) in 24 heavily pre-treated patients with different types of blood cancer. 4SC-202 was well tolerated with few and/or manageable adverse events. Positive signs of anti-tumor efficacy were observed with one complete remission for 28 months and one partial responder for 8 months. Findings also exhibited disease control in 83% of the patients and long-term stabilization in 50% of patients.

WO 2006/097474 A1 describes certain N-sulphonylpyrrole derivatives and their medical utility.

WO 2009/112522 A1 describes salts of certain N-sulphonylpyrrole derivatives and their medical utility.

### BRIEF DESCRIPTION OF THE FIGURES

FIG. 1: Results of Preclinical murine in vivo experiment with CD38 cells (see Example A): a) tumor growth—X-axis is days, Y-axis is tumor volume in mm³, groups are from top to bottom: vehicle (upside down triangle), anti-PD-1 AB (solid square), 20 mg/kg BID 4SC-202 (solid diamond), 20 mg/kg BID 4SC-202+anti-PD-1 AB (solid circle), 60 mg/kg BID 4SC-202 (open circle), 60 mg/kg BID 4SC-202+anti-PD-1 AB (solid triangle); b) Kaplan-Meier plot survival—X-axis is days, Y-axis is survival in %, groups are from left to right: vehicle (solid line), anti-PD-1 AB (dashed line), 60 mg/kg BID 4SC-202 (dotted line), 60 mg/kg BID 4SC-202+anti-PD-1 AB (dashed/dotted line).

FIG. 2: Results of Preclinical murine in vivo experiment with CT26 cells (see Example A): a) tumor growth—X-axis is days, Y-axis is tumor volume in mm³, groups are from top to bottom: vehicle (solid circle), anti-PD-L1 AB (open circle), 20 mg/kg BID 4SC-202 (solid triangle), 20 mg/kg BID 4SC-202+anti-PD-1 AB (upside down triangle; b) Kaplan-Meier plot survival—X-axis is days, Y-axis is survival in %, groups are from left to right: vehicle (solid line), anti-PD-L1 AB (dashed line), 20 mg/kg BID 4SC-202 (dotted line), 20 mg/kg BID 4SC-202+anti-PD-1 AB (dashed/dotted line).

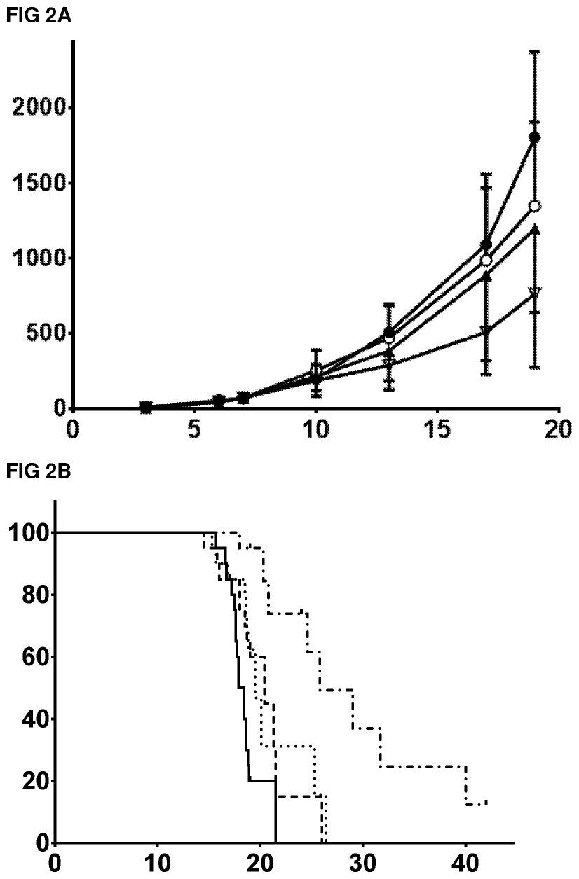

FIG. 3: Results of the food effect dog study (Example B), for a) fasted and b) fed conditions. In each case, x-axis is time (h) and y-axis is concentration observed (μg/L).

### DESCRIPTION OF THE INVENTION

It has now been found unexpectedly that the combination therapy utilizing an HDAC inhibitor of the present invention with at least one immune checkpoint modulator shows beneficial efficacy compared with immune checkpoint modulator monotherapy. Moreover, the specific dosing as detailed herein shows particularly beneficial effects, such as allowing for a decreased dosage of the HDAC inhibitor and thus favorable tolerability of the treatment. These effects are particularly pronounced in certain specific cancers, as detailed herein.

Certain embodiments of the present invention are listed in the following items:
1. Use of an HDAC inhibitor of the below general formula I or a salt or solvate thereof for the manufacture of a medicament for the treatment of cancer to be used in combination with at least one immune checkpoint modulator,

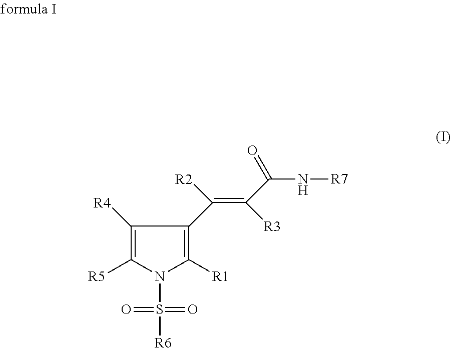

in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene,
either Q1 is substituted by R61 and/or R62, and is Aa1, Hh1, Ha1, Ha2, Ha3, Ha4 or Ah1, or Q1 is unsubstituted, and is Ha2, Ha3 or Ha4,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, —U-T3-N(R613)R614, -T4-Het3, or —V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups, which are selected independently from a group consisting of phenyl and naphthyl, and which are linked together via a single bond,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cyc1, in which Cyc1 is a ring system of formula Ia

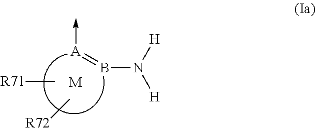

in which
A and B are C (carbon),
R71 and R72 are independently hydrogen, halogen, 1-4C-alkyl, or 1-4C-alkoxy, M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur.
2. The use according to item 1, wherein the HDAC inhibitor is (E)-N-(2-aminophenyl)-3-(1-((4-(1-methyl-1H-pyrazol-4-yl)phenyl)sulfonyl)-1H-pyrrol-3-yl)acrylamide (also known as 4SC-202).
3. The use according to item 2, wherein (E)-N-(2-aminophenyl)-3-(1-((4-(1-methyl-1H-pyrazol-4-yl)phenyl)sulfonyl)-1H-pyrrol-3-yl)acrylamide is administered in a dose of 150 to 250, particularly 175 to 225, more particularly 190 to 210, even more particularly 195 to 205, yet even more particularly about 200 mg/day,
   or, in an alternative embodiment, (E)-N-(2-aminophenyl)-3-(1-((4-(1-methyl-1H-pyrazol-4-yl)phenyl)sulfonyl)-1H-pyrrol-3-yl)acrylamide is administered in a dose of 80 to 120, particularly 90 to 110, more particularly 95 to 105, even more particularly about 100 mg/day,
   or, in another alternative embodiment, (E)-N-(2-aminophenyl)-3-(1-((4-(1-methyl-1H-pyrazol-4-yl)phenyl)sulfonyl)-1H-pyrrol-3-yl)acrylamide is administered in a dose of 300 to 500, particularly 350 to 450, more particularly 375 to 425, even more particularly 390 to 410, yet even more particularly 395 to 405, yet even more particularly about 400 mg/day,
   wherein the aforementioned daily doses are optionally administered in two portions (each half of the aforementioned amounts), twice daily, particularly one each in the morning and evening (wherein particularly the evening dose is administered 10-14, more particularly 11-13, even more particularly about 12 hours after the morning dose).
4. The use according to any one of items 1 to 3, wherein the at least one immune checkpoint modulator is selected from the group consisting of a) inhibitors of anti-inflammatory immune checkpoints including PD-1, CTLA-4, A2AR, B7-H3 (also known as CD276), B7-H4 (also known as VTCN1), BTLA, IDO, KIR, LAG3, TIM-3, VISTA (V-domain Ig suppressor of T cell activation) and their respective ligands including PD-L1, PD-L2, and galectin), and b) agonists of pro-inflammatory immune checkpoints including CD27, CD40, OX40, GITR, CD137, CD28, ICOS, and their respective ligands, including CD70 CD80, CD86, CD40L, CD137 ligand, OX40L, GITR ligand and ICOSL, more particularly selected from the group consisting of PD-1 inhibitors, PD-L1 inhibitors, CTLA-4 inhibitors, IDO inhibitors and LAG3 inhibitors,
   even more particularly selected from the group consisting of PD-1 inhibitors and PD-L1 inhibitors.

In particular embodiments, the immune checkpoint modulator is a molecule for which binding to an immune checkpoint is determinable in an ELISA assay, in particular with an EC₅₀ 250 nM or lower, more particularly 100 nM or lower, even more particularly 75 nM or lower. A particular ELISA useable in this context, in particular for biologicals, more particularly for antibodies, is the following assay:

Material & Methods:
Immune checkpoint modulator
recombinant Immune checkpoint (particularly human)
capture antibody specific to immune checkpoint modulator, coupled to horse radish peroxidase (HRP)
ELISA Plate 96 Well high binding (Greiner #655061)
PBS (e.g. Gibco #21300-058)
BSA (e.g. Sigma # A3733)
Tween20 (e.g. Sigma # P1379)
TMB (3,3′,5,5′-Tetramethylbenzidin) ELISA substrate (e.g. 1-Step™ Ultra TMB-ELISA Substrate Solution by Thermo #34029)
Blocking solution: 1% BSA in PBS
Washing solution: PBS with 0.05% Tween
Stopping solution: sulphuric acid 250 mM
1. dissolve 1 μg/mL recombinant Immune checkpoint in PBS, add 100 μL of said solution per well to ELISA 96 well plates, incubate the plates for 12-24 h at 4° C. (to coat the wells with Immune checkpoint)
2. subsequently, remove solution and wash each well twice with 200 μL washing solution
3. subsequently add 200 μL of blocking solution per well, incubate at room temperature (about 22° C.) for 1 hour
4. subsequently, remove blocking solution and wash each well with 1×200 μL washing solution
5. subsequently, add 100 μL serial dilutions of Immune checkpoint modulator in PBS with 1% BSA to the respective wells (a particularly suitable range of serial dilutions could comprise 1 μM, 0.5 μM, 0.25 μM, 0.125 μM, 0.06 μM, 0.03 μM, 0.015 μM, 8 nM, 4 nM and 2 nM), incubate for 1 hour at room temperature
6. subsequently, remove supernatant and wash each well with 4× with 200 μL washing solution
7. subsequently, add 100 μL per well of capture antibody solution in PBS with 1% BSA (particularly 50-150, more particularly 75-125, even more particularly 90-110, yet even more particularly about 100 ng/mL) (e.g. goat anti human IgG-HRP), incubate 45 min at room temperature
8. subsequently, remove supernatant and wash each well with 6× with 200 μL washing solution
9. subsequently, add 100 μL per well of TMB substrate
10. upon sufficient completion of the reaction (i.e. when a color gradient is visible between the different checkpoint modulator dilutions, typically 5-20, particularly 5-15 minutes, more about 10 minutes after substrate addition), add 100 μL 250 mM sulfuric acid per well (to stop the reaction)
11. subsequently, measure absorption at 450 nm in a suitable plate reader (e.g. Tecan Sunrise)
12. plot data as relative absorption at 450 nm versus Immune checkpoint modulator concentration and calculate EC₅₀ values using a suitable curve fit to a suitable pharmacological model (e.g. Emax model) using a suitable software (e.g. Graphpad Prism).

For instance, recombinant Immune checkpoint and capture antibody can be, in specific forms of the assay: recombinant human PD-1 (e.g. R&D #8986-PD-100), recombinant human PD-L1 (e.g. acro # PD1-H5229), recombinant human CTLA-4 (e.g. abcam # ab169909); anti human IgG HRP (e.g. Sigma # A0170-1 ML).

Alternative competition format: The above assay procedure can be run in the competition format which is e.g. suitable to determine binding of small molecule Immune checkpoint modulators.

In step 5, add serial dilutions of Immune checkpoint modulators—for small molecules, suitable dilutions could comprise 100 μM, 50 μM, 25 μM, 12.5 μM, 6 μM, 3 μM, 1.5 μM, 800 nM, 400 nM, 200 nM, 100 nM, 50 nM, 25 nM, 12.5 nM, 6 nM, 3 nM and 1.5 nM—and add recombinant Immune checkpoint ligand (e.g. in serial dilutions in a matrix pattern versus serial dilutions of Immune checkpoint modulators to determine suitable concentration of Immune checkpoint ligand, which may be in the a similar range as the aforementioned dilutions). In step 7, the capture antibody is specific for the recombinant Immune checkpoint ligand. Suitable IC50 values for small molecules are 500 nM or lower, particularly 250 nM or lower, more particularly 100 nM or lower, even more particularly 75 nM or lower, yet even more particularly 50 nM or lower.

The immune checkpoint modulators may be small molecules (having a molecular weight of about 600 or lower, particularly 500 or lower, more particularly 400 or lower) or biologicals (as used herein such as antibodies, modified antibodies, antibody fragments and scaffold proteins).

In particular embodiments, the at least one immune checkpoint modulator is an antibody, more particularly a human antibody or a humanized antibody.

In particular embodiments, the at least one immune checkpoint modulator is selected from the group consisting of Ipilimumab, pembrolizumab, avelumab, nivolumab, durvalumab, tremelimumab, BCD-100 (Biocad), PDR-001 (Novartis), REGN-2810 (Regeneron), Camrelizumab (Shanghai Hengrui), SHR-1210 (Incyte), AGEN-2034 (Agenus), BGBA-317 (BeiGene), BMS-936559 (ViiV Healthcare), CX-072 (CytomX), CX-188 (CytomX), GNS-1480 (Genosco/Yuhan), IBI-308 (Eli Lilly/Innovent), JNJ-63723283 (J&J), JS-001 (Shanghai Junshi), MEDI-0680 (Medlmmune), AMP-224 (Medlmmune), BGB-A317 (BeiGene/Celgene), BI-754091 (Boehringer), CA-170 (Curis/Aurigene), CBT-501 (CBT Pharma), Genolimzunab (Genor), CBT-502 (CBT Pharma), FAZ-053 (Novartis), GLS-010 (Harbin/Wuxi/Arcus), AB122 (Harbin/Wuxi/Arcus), LY-3300054 (Eli Lilly), KN-035 (AlphaMab), M-7824 (Merck KGaA), MAG-012 (MacroGenics), MGD-013 (MacroGenics), PF-06801591 (Pfizer), SHR-1316 (Jiangsu Hengrui), TSR-042 (Tesaro), CS-1001 (CStone Pharma), HLX-10 (Shanghai Henlius), MCLA-145 (Merus/Incyte), AM-0001 (ARMO Bio), AVA-004 (Avacta), STI-al014 (Lee's Pharma/Sorrento), hAb-21 (Suzhou Stainwei), AK103 (Akeso Bio), AK104 (Akeso Bio), AK105 (Akeso Bio), AK106 (Akeso Bio), AK112 (Akeso Bio), BBI (Boston Biomedicals), BH-2922 (Beijing Hanmi), BH-2941 (Beijing Hanmi), BH 2950 (Beijing Hanmi), CA-327 (Curis/Aurigene), CBA-0710 (Sorrento), CK-301 (TG therapeutic), ENUM-244C8 (Enumeral), FS-118 (F-star Alpha/Merck KGaA), HTI-1316 (Hengrui Therapeutics), IKT-201 (Icell Kealex), IKT-202 (Icell Kealex), vaccinia virus expressing checkpoint inhibitor (Icell Kealex), JS-003 (Shanghai Junshi), JTX-4014 (Jounce/Celgene), KD033 (Kadmon/Jinghua Pharma), KY-1003 (Kymab), MCLA-134 (Merus), MSB-2311 (MABSPACE Bio), PRS-332 (Pieris/Servier), RXI-762 (Rxi Pharmaceuticals), SN-PD07 (Synovel), SN-PDL01 (Synovel), STI-A1110 (Sorrento/Servier), XmAb20717 (Xencor), AT16201 (AIMM), HLX-20 (Shanghai Henlius), IMM-1802 (ImmuneOnco Biopharma Shanghai), IMM-25 (ImmuneOnco Biopharma Shanghai), IMM-2502 (ImmuneOnco Biopharma Shanghai), IMM-2503 (ImmuneOnco Biopharma Shanghai), IMM-2504 (ImmuneOnco Biopharma Shanghai), CDX-1127 (Celldex Therapeutics NKTR-214 (Nektar Therapeutics), MED10562 (AstraZeneca), MED16469 (AstraZeneca), MED16383(AstraZeneca), MGA271 (MacroGenics), Lirilumab, and atezolizumab, more particularly the immune checkpoint modulator is selected from the group consisting of Ipilimumab, pembrolizumab, avelumab, nivolumab, durvalumab, tremelimumab, Genolimzunab, Lirilumab, and atezolizumab, even more particularly the immune checkpoint modulator is selected from the group consisting of pembrolizumab, avelumab and nivolumab, yet even more particularly pembrolizumab.
5. The use according to item 4, wherein pembrolizumab is administered in a dose of 2 mg/kg, or in a dose of 200 mg, nivolumab is administered in a dose of 3 mg/kg, or in a dose of 240 mg or in a dose of 480 mg, ipilimumab is administered in a dose of 3 mg/kg or in a dose of 10 mg/kg, avelumab is administered in a dose of 10 mg/kg, atezolizumab is administered in a dose of 1200 mg, durvalumab is administered in a dose of 1500 mg, tremelimumab is administered in a dose of 1 mg/kg, in a dose of 75 mg.

Particularly, pembrolizumab is administered in a dose of 2 mg/kg, more particularly every three weeks, or alternatively in a (fixed) dose of 200 mg, more particularly every three weeks. Particularly, nivolumab is administered in a dose of 3 mg/kg, more particularly every two weeks, or alternatively in a (fixed) dose of 240 mg, more particularly every two weeks, or alternatively in a (fixed) dose of 480 mg, more particularly every four weeks.

Particularly, ipilimumab is administered in a dose of 3 mg/kg, more particularly every three weeks, or alternatively in a dose of 10 mg/kg, more particularly every three weeks.

Particularly, avelumab is administered in a dose of 10 mg/kg, more particularly every two weeks.

Particularly, atezolizumab is administered in a (fixed) dose of 1200 mg, more particularly every three weeks.

Particularly, durvalumab is administered in a (fixed) dose of 1500 mg, more particularly every four weeks.

Particularly, tremelimumab is administered in a dose of 1 mg/kg, more particularly every four weeks, or in a (fixed) dose of 75 mg, more particularly every four weeks.

As used above in the context of the administration of the immune checkpoint modulator, the term fixed dose is meant to refer to a dose that is equally administered to every patient, i.e. that does not take into account the respective patient's body weight.

The at least one immune checkpoint modulator is to be administered in a dose that is typically used by the physician for the respective immune checkpoint modulator, in particular the dose approved by the respective governmental authorities. Typically, immune checkpoint modulators that are biologicals (such as antibodies, modified antibodies, antibody fragments and scaffold proteins) are to be administered only on day one of a treatment cycle, which may be a particular treatment cycle as described herein. This is due to their long half-life in the patient's system.

The term antibody in the meaning of the invention comprises all antibodies, antibody fragments, and derivatives thereof that are capable of binding to an antigen, in this case the immune checkpoint. This encompasses the complete monoclonal antibodies and also the epitope-binding fragments of these antibodies. In this connection, the epitope binding fragments (also referred to herein as antibody fragments or antibody derivatives) comprise all regions of the antibody that are capable of binding to the antigen. Examples of particular antibody fragments in accordance with the invention comprise, but expressly are not limited to, Fab, Fab′, F(ab′)2, Fd, individual chain (single chain) variable fragments (scFv), single-chain antibodies, disulfide-linked variable fragments (sdFv), and fragments that either contain a variable region of the light chain (V_{L}) or a variable region of the heavy chain (V_{H}). Moreover, they include recombinantly prepared antibodies, such as diabodies, and tetrabodies.

Antibody fragments contain the variable regions either alone or in combination with further regions that are selected from the hinge region and the first, second and third regions of the constant region (C_{H}1, C_{H}2, C_{H}3). Also, the term antibody comprises chimeric antibodies in which different regions of the antibody originate from different species, for example, antibodies with a murine variable region combined with a human constant region.

Antibody fragments are optionally linked with each other by a linker. The linker comprises a short (particularly 10 to 20 amino acid residues), flexible peptide sequence that is selected such that the antibody fragment has such a three dimensional folding of VL and VH that it exhibits the antigen specificity of the complete antibody. Particular linkers are glycine-serine linkers with the structure (GlyₓSer_{y}) with x and y selected from 1 to 10, particularly 3 to 5.

Moreover, particular linkers are comprised of a peptide sequence that can increase the protease resistance of the antibody derivatives.

As used herein, scaffolds are protein structures possessing the ability to specifically bind to immune checkpoints and show comparable binding strength and selectivity as an antibody binding to said immune checkpoints.
6. The use according to any one of items 1 to 5, wherein the HDAC inhibitor is administered on days 1 to 14, or on days 1, 3, 5, 7, and 9, and the at least one immune checkpoint modulator is administered on day 1 in a 21-day treatment cycle, or
   wherein the HDAC inhibitor is administered on days 1 to 7, or on days 1, 3, and 5, and the at least one immune checkpoint modulator is administered on day 1 in a 14-day treatment cycle.

The treatment cycles as described herein can be repeated one or more times, and typically are repeated as often as necessary, which is typically to be determined by the physician, e.g. based on the disease state (progressive disease, stable disease, tumor regression, etc.), and/or the tolerability of the treatment.
7. The use according to any one of items 1 to 6, wherein the treatment comprises a first treatment cycle wherein only the HDAC inhibitor is administered (and no immune checkpoint modulator is administered) prior to administering the HDAC inhibitor and the immune checkpoint modulator (in combination).
8. The use according to any one of items 1 to 7, wherein the treatment comprises administering the HDAC inhibitor to the patient having said cancer in the non-fasted state.

In an alternative embodiment, the treatment comprises administering the HDAC inhibitor to the patient having said cancer in the fasted state, which is in particular that the patient having said cancer does not receive food 2 hours before and 1 hour after each treatment.
9. The use according to any one of items 1 to 8, wherein said cancer is a solid tumor;
   or alternatively a refractory, non-responding or relapsed to immune checkpoint modulator therapy (wherein particularly refractory means no stabilization is achieved with immune checkpoint modulator therapy, non-responding means the best response achieved with immune checkpoint modulator therapy is stable disease for 6 months or less followed by disease progression, relapsed means temporary response shrinkage followed by disease progression, wherein disease status including response, progression, stabilization is determined according to RECIST or immune related RECIST (irRECIST) criteria version-reference Eisenhauer et al. 2009 Eur J Cancer, 45, 228-247; Nishino M et al., Clin Cancer Res. 2013 Jul. 15; 19(14):3936-4 3);
   or alternatively an immunologically cold tumor (means in particular that it is insufficiently infiltrated by T-cells; not sufficiently visible by the immune system; exhibiting an insufficient amount of tumor antigen presentation, in particular proteins of the tumor antigen presentation machinery, e.g. via MHC I or II—this can be determined e.g. via immune histochemistry, methods of which are well known in the field, such as for example the methods described in Arpita Kabiraj et al., Int J Biol Med Res. 2015; 6(3): 5204-5210 and references therein to the specific methods), particularly, this is a tumor with an immune cell infiltration corresponding to an immunoscore of 0-2, more particularly 0 or 1, more particularly 1, or in alternative embodiments an immunoscore of 2-4, 2-3 or 4,
   or alternatively suitable for treatment with an immune checkpoint modulator, wherein this is particularly a cancer for which an immune checkpoint modulator therapy is approved, i.e. that has received market approval by the regulatory authorities in at least one country,
   or in particular a cancer selected from the group consisting of
   melanoma (in particular ocular and uveal, but also including skin melanoma), head and neck, renal, NSCLC, microsatellite-instable carcinoma (lynch syndrome, in particular gastroesophageal and colorectal), urothelial carcinoma including bladder, merkel cell carcinoma, hodgkin lymphoma, gastric, oesophageal, non-hodgkin lymphoma, SCLC, sarkoma, mesothelioma, glioblastoma, microsatellite stable (in particular gastroesophageal and colorectal), pancreas, HCC, prostata, basal cell carcinoma, CTCL, and squamous cell carcinoma;
   more particularly melanoma (in particular ocular and uveal, but also including skin melanoma), head and neck, renal, NSCLC, microsatellite-instable carcinoma (lynch syndrome, in particular gastroesophageal and colorectal), urothelial carcinoma including bladder, merkel cell carcinoma, hodgkin lymphoma), gastric, oesophageal, non-hodgkin lymphoma, SCLC, sarkoma, mesothelioma, glioblastoma, microsatellite stable (in particular gastroesophageal and colorectal), pancreas, and HCC;
   even more particularly melanoma (in particular ocular and uveal, but also including skin melanoma), head and neck, renal, NSCLC, microsatellite-instable carcinoma (lynch syndrome, in particular gastroesophageal and colorectal), urothelial carcinoma including bladder, merkel cell carcinoma, hodgkin lymphoma, gastric, oesophageal, non-hodgkin lymphoma, and SCLC;
   yet even more particularly melanoma (in particular ocular and uveal, but also including skin melanoma), head and neck, renal, NSCLC, microsatellite-instable carcinoma (lynch syndrome, in particular gastroesophageal and colorectal), urothelial carcinoma including bladder, merkel cell carcinoma, and hodgkin lymphoma.
   In a particular embodiment the cancer is cutaneous melanoma.

The number of immune cells and/or its ratio versus the total cell number in a tumor in the context of the present invention is determinable by standard methods known to the skilled person and in particular embodiments determinable in a formalin-fixed paraffin-embedded tumor sample obtainable from the patient by

1) cutting a 5-101M slicea of said sample,

2) fixing the slices in 4% PF,

3) rinsing twice in PBS for 2 minutes,

4) adding commercially available serum (5% in PBS) and incubating for 20 minutes,

5) adding a primary commercially available antibody against CD3+ or CD8+ and incubating for 60 min (dilution of 51 μg/ml in PBS),

6) rinsing twice in PBS for 2 minutes,

7) adding a secondary biotinylated antibody (binding to the constant region of the primary antibody) and incubating for 30 min,

8) rinsing twice in PBS for 2 minutes,

9) add streptavidin-peroxidase (e.g. Jackson Immunoresearch), incubate for 30 min,

10) rinsing twice in PBS for 2 minutes,

11) add developer (e.g. AEC Substrate Chromogen Ready-to-Use, Dako # K3464), particularly until sufficiently stained (typically observe development under microscope, typically for 5 min)

12) rinse with water

13) counterstain with commercially available HTX solution

14) mount in water-based mounting media (e.g. DAKO)

15) determine CD3+ or CD8+ cell number

16) optionally determine cell number ratio by dividing CD3+ or CD8+ cell number by total cell number in tumor volume (e.g. based on typical cell numbers in said specific cancer type);

Or by the following assay

For each tumor sample, stain 2 slides are using an automated immunohistochemistry staining instrument (BenchMark XT, Ventana): one with CD3 and one with CD8 ready-to-use monoclonal antibodies (HalioDx).

Perform staining with ultraView Universal DAB Detection Kit (Ventana), followed by counterstaining (Bluing Reagent, Ventana).

Wash stained slides, dehydrate, mount and coverslip.

Obtain digital images of stained slides using a whole slide scanner (Nanozoomer XR, Hamamatsu), and analyze by a software program (Immunoscore®Analyzer, HalioDx) or count to determine cell numbers and optionally determine ratio as above.

(Optional: One separate control slide with 3 external controls—1 negative tissue (placenta) and 2 positive (1 tissue: tonsil and a cell line pellet)—is processed identically in each IHC run, and allows monitoring of the staining and scanning steps.)
10. The use according to any one of items 1 to 9, wherein the patient having said cancer has received at least one prior systemic treatment against said cancer,
   particularly at least one prior systemic chemotherapeutic treatment against said cancer, more particularly at least one prior systemic treatment comprising the administration of at least one immune checkpoint modulator,
   or more particularly at least one prior systemic treatment comprising the administration of at least one immune checkpoint modulator, even more particularly at least one immune checkpoint modulator selected from the group consisting of pembrolizumab, avelumab and nivolumab, yet even more particularly Pembrolizumab or Nivolumab.

In certain embodiments of the present invention, said prior systemic chemotherapeutic treatment is a treatment of administrating one or more chemotherapeutic agents systemically, such chemotherapeutic agent may be used alone or in combination with further agents.

In certain embodiments of the present invention, said patient having said cancer has received at least one prior systemic treatment comprising the administration of at least one immune checkpoint modulator, particularly at least one inhibitor of anti-inflammatory immune checkpoints, more particularly of PD-1, even more particularly Pembrolizumab or Nivolumab, against said cancer and said patient was a non-responder or said cancer was refractory or relapsed to said at least one prior systemic treatment.

In a certain specific embodiment of the present invention, said patient having said cancer has received at least one prior systemic treatment comprising the administration of Nivolumab in combination with Ipilimumab against said cancer and said patient was a non-responder or said cancer was refractory or relapsed to said at least one prior systemic treatment.
11. The use according to any one of items 1 to 10, wherein said cancer is resistant to immune checkpoint modulator therapy,
   particularly to therapy with at least one immune checkpoint modulator, even more particularly at least one immune checkpoint modulator selected from the group consisting of pembrolizumab, avelumab and nivolumab, yet even more particularly Pembrolizumab or Nivolumab.
12. An HDAC inhibitor of the general formula I as explained to, e.g., in item 1, or a salt or solvate thereof in combination with at least one immune checkpoint modulator for use in the treatment of cancer.
13. The HDAC inhibitor of general formula I or a salt or solvate thereof in combination with at least one immune checkpoint modulator for use in the treatment of cancer according to item 12, wherein the HDAC inhibitor is (E)-N-(2-aminophenyl)-3-(1-((4-(1-methyl-1H-pyrazol-4-yl)phenyl)sulfonyl)-1H-pyrrol-3-yl)acrylamide (also known as 4SC-202).
14. The HDAC inhibitor of general formula I or a salt or solvate thereof in combination with at least one immune checkpoint modulator for use in the treatment of cancer according to item 13, wherein (E)-N-(2-aminophenyl)-3-(1-((4-(1-methyl-1H-pyrazol-4-yl)phenyl)sulfonyl)-1H-pyrrol-3-yl)acrylamide is administered in a dose of 150 to 250 mg/day, wherein the aforementioned daily doses are optionally administered in two portions, twice daily.
15. The HDAC inhibitor of general formula I or a salt or solvate thereof in combination with at least one immune checkpoint modulator for use in the treatment of cancer according to any one of items 12 to 14, wherein the at least one immune checkpoint modulator is selected from the group consisting of a) inhibitors of anti-inflammatory immune checkpoints including PD-1, CTLA-4, A2AR, B7-H3 (also known as CD276), B7-H4 (also known as VTCN1), BTLA, IDO, KIR, LAG3, TIM-3, VISTA (V-domain Ig suppressor of T cell activation) and their respective ligands including PD-L1, PD-L2, and galectin), and b) agonists of pro-inflammatory immune checkpoints including CD27, CD40, OX40, GITR, CD137, CD28, ICOS, and their respective ligands, including CD70 CD80, CD86, CD40L, CD137 ligand, OX40L, GITR ligand and ICOSL.
16. The HDAC inhibitor of general formula I or a salt or solvate thereof in combination with at least one immune checkpoint modulator for use in the treatment of cancer according to any one of items 12 to 15, wherein the at least one immune checkpoint modulator is selected from the group consisting of Ipilimumab, pembrolizumab, avelumab, nivolumab, durvalumab, tremelimumab, BCD-100 (Biocad), PDR-001 (Novartis), REGN-2810 (Regeneron), Camrelizumab (Shanghai Hengrui), SHR-1210 (Incyte), AGEN-2034 (Agenus), BGBA-317 (BeiGene), BMS-936559 (ViiV Healthcare), CX-072 (CytomX), CX-188 (CytomX), GNS-1480 (Genosco/Yuhan), IBI-308 (Eli Lilly/Innovent), JNJ-63723283 (J&J), JS-001 (Shanghai Junshi), MEDI-0680 (Medlmmune), AMP-224 (Medlmmune), BGB-A317 (BeiGene/Celgene), BI-754091 (Boehringer), CA-170 (Curis/Aurigene), CBT-501 (CBT Pharma), Genolimzunab (Genor), CBT-502 (CBT Pharma), FAZ-053 (Novartis), GLS-010 (Harbin/Wuxi/Arcus), AB122 (Harbin/Wuxi/Arcus), LY-3300054 (Eli Lilly), KN-035 (AlphaMab), M-7824 (Merck KGaA), MAG-012 (MacroGenics), MGD-013 (MacroGenics), PF-06801591 (Pfizer), SHR-1316 (Jiangsu Hengrui), TSR-042 (Tesaro), CS-1001 (CStone Pharma), HLX-10 (Shanghai Henlius), MCLA-145 (Merus/Incyte), AM-0001 (ARMO Bio), AVA-004 (Avacta), STI-a1014 (Lee's Pharma/Sorrento), hAb-21 (Suzhou Stainwei), AK103 (Akeso Bio), AK104 (Akeso Bio), AK105 (Akeso Bio), AK106 (Akeso Bio), AK112 (Akeso Bio), BBI (Boston Biomedicals), BH-2922 (Beijing Hanmi), BH-2941 (Beijing Hanmi), BH 2950 (Beijing Hanmi), CA-327 (Curis/Aurigene), CBA-0710 (Sorrento), CK-301 (TG therapeutic), ENUM-244C8 (Enumeral), FS-118 (F-star Alpha/Merck KGaA), HTI-1316 (Hengrui Therapeutics), IKT-201 (Icell Kealex), IKT-202 (Icell Kealex), vaccinia virus expressing checkpoint inhibitor (Icell Kealex), JS-003 (Shanghai Junshi), JTX-4014 (Jounce/Celgene), KD033 (Kadmon/Jinghua Pharma), KY-1003 (Kymab), MCLA-134 (Merus), MSB-2311 (MABSPACE Bio), PRS-332 (Pieris/Servier), RXI-762 (Rxi Pharmaceuticals), SN-PD07 (Synovel), SN-PDL01 (Synovel), STI-A1110 (Sorrento/Servier), XmAb20717 (Xencor), AT16201 (AIMM), HLX-20 (Shanghai Henlius), IMM-1802 (ImmuneOnco Biopharma Shanghai), IMM-25 (ImmuneOnco Biopharma Shanghai), IMM-2502 (ImmuneOnco Biopharma Shanghai), IMM-2503 (ImmuneOnco Biopharma Shanghai), IMM-2504 (ImmuneOnco Biopharma Shanghai), CDX-1127 (Celldex Therapeutics NKTR-214 (Nektar Therapeutics), MEDI0562 (AstraZeneca), MED16469 (AstraZeneca), MED16383(AstraZeneca), MGA271 (MacroGenics), Lirilumab, and atezolizumab.
17. The HDAC inhibitor of general formula I or a salt or solvate thereof in combination with at least one immune checkpoint modulator for use in the treatment of cancer according to item 16, wherein pembrolizumab is administered in a dose of 2 mg/kg, or in a dose of 200 mg, nivolumab is administered in a dose of 3 mg/kg, or in a dose of 240 mg or in a dose of 480 mg, ipilimumab is administered in a dose of 3 mg/kg or in a dose of 10 mg/kg, avelumab is administered in a dose of 10 mg/kg, atezolizumab is administered in a dose of 1200 mg, durvalumab is administered in a dose of 1500 mg, tremelimumab is administered in a dose of 1 mg/kg, in a dose of 75 mg.
18. The HDAC inhibitor of general formula I or a salt or solvate thereof in combination with at least one immune checkpoint modulator for use in the treatment of cancer according to any one of items 12 to 17, wherein the HDAC inhibitor is administered on days 1 to 14, or on days 1, 3, 5, 7, and 9, and the at least one immune checkpoint modulator is administered on day 1 in a 21-day treatment cycle, or
   wherein the HDAC inhibitor is administered on days 1 to 7, or on days 1, 3, and 5, and the at least one immune checkpoint modulator is administered on day 1 in a 14-day treatment cycle.
19. The HDAC inhibitor of general formula I or a salt or solvate thereof to be used in combination with at least one immune checkpoint modulator for use in the treatment of cancer according to any one of items 12 to 18, wherein the treatment comprises a first treatment cycle wherein only the HDAC inhibitor is administered prior to administering the HDAC inhibitor and the immune checkpoint modulator.
20. The HDAC inhibitor of general formula I or a salt or solvate thereof in combination with at least one immune checkpoint modulator for use in the treatment of cancer according to any one of items 12 to 19, wherein the treatment comprises administering the HDAC inhibitor to the patient having said cancer in the non-fasted state.
21. The HDAC inhibitor of general formula I or a salt or solvate thereof in combination with at least one immune checkpoint modulator for use in the treatment of cancer according to any one of items 12 to 20, wherein said cancer is a solid tumor.
22. The HDAC inhibitor of general formula I or a salt or solvate thereof in combination with at least one immune checkpoint modulator for use in the treatment of cancer according to any one of items 12 to 21, wherein said cancer is selected from the group consisting of melanoma including ocular, uveal and skin melanoma, head and neck, renal, NSCLC, microsatellite-instable carcinoma including lynch syndrome including gastroesophageal and colorectal, urothelial carcinoma including bladder, merkel cell carcinoma, hodgkin lymphoma, gastric, oesophageal, non-hodgkin lymphoma, SCLC, sarkoma, mesothelioma, glioblastoma, microsatellite stable including gastroesophageal and colorectal, pancreas, HCC, prostata, basal cell carcinoma, CTCL, and squamous cell carcinoma.
23. The HDAC inhibitor of general formula I or a salt or solvate thereof in combination with at least one immune checkpoint modulator for use in the treatment of cancer according to any one of items 12 to 22, wherein said cancer is a refractory, non-responding or relapsed to immune checkpoint modulator therapy.
24. The HDAC inhibitor of general formula I or a salt or solvate thereof in combination with at least one immune checkpoint modulator for use in the treatment of cancer according to any one of items 12 to 23, wherein the patient having said cancer has received at least one prior systemic treatment against said cancer.
25. The HDAC inhibitor of general formula I or a salt or solvate thereof in combination with at least one immune checkpoint modulator for use in the treatment of cancer according to any one of items 12 to 24, wherein said cancer is resistant to immune checkpoint modulator therapy.
26. Other embodiments relate to methods of treatment of a patient in need thereof in accordance with any of the items 1 to 24.

In particular embodiments of the present invention, the patient is a human patient. The patient is a subject suffering from cancer, in particular the specific cancer types described herein.

The HDAC inhibitor is meant to be inclusive of the respective salts, solvates and hydrates.

As used herein, immune checkpoints are molecules (e.g. receptors on the membrane of T cells or their respective ligands) modulating the immune system, for instance attenuating (anti-inflammatory) or increasing (pro-inflammatory) an immune response. Many cancers protect themselves from the immune system by inhibiting the signal s leading to the destruction of tumor cells. An immune checkpoint modulator is particularly an agent that aims to alter the immune system of the patient, in particular increase T cell response, to control, stabilize or reduce tumor growth. They have a potential for a complete eradication of the disease.

In the present invention, the HDAC inhibitor and the at least one immune checkpoint modulator are typically to be administered in therapeutically effective amounts.

The HDAC inhibitor is in a first aspect (aspect A) a compound of formula I

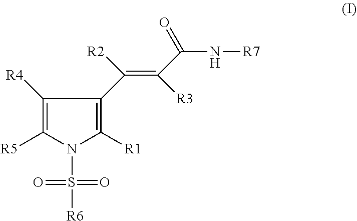

in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene, either
Q1 is substituted by R61 and/or R62, and is Aa1, Hh1, Ha1, Ha2, Ha3, Ha4 or Ah1,

or
Q1 is unsubstituted, and is Ha2, Ha3 or Ha4,

in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, —U-T3-N(R613)R614, -T4-Het3, or —V-T5-Het4, in which
T2 is a bond or 1-4C-alkylene,
R61 1 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups,
   which are selected independently from a group consisting of phenyl and naphthyl, and
   which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cyc1, in which Cyc1 is a ring system of formula Ia

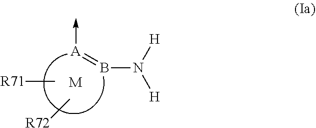

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur,

and the salts of these compounds.

The HDAC inhibitor is in a second aspect (aspect B), which is an embodiment of aspect A, a compound of formula I, in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene,

either
Q1 is substituted by R61 and/or R62, and is Aa1, Hh1, Ha1, Ha2, Ha3 or Ah1,
or Q1 is unsubstituted, and is Ha2 or Ha3,

in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, or —U-T3-N(R613)R614, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R614 is hydrogen or 1-4C-alkyl,
or R613 and R614 together and with inclusion of the nitrogen atom, to which they are bonded, form a heterocyclic ring Het2, in which Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
   which are selected independently from a group consisting of phenyl and naphthyl,
   and
   which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cyc1, in which Cyc1 is a ring system of formula Ia

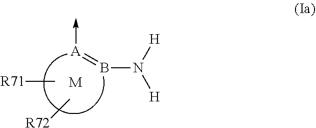

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which
Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,

and the salts of these compounds.

The HDAC inhibitor is in a third aspect (aspect C), which is also an embodiment of aspect A, a compound of formula I,

in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond, or 1-4C-alkylene,

either
Q1 is substituted by R61 and/or R62, and is Aa1, Hh1, Ha1, Ha2, Ha3 or Ah1,
or Q1 is unsubstituted, and is Ha2 or Ha3,

in which
R61 is 1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, or -T2-N(R611)R612, in which
T2 is a bond or 1-4C-alkylene,
R611 and R612 are independently hydrogen or 1-4C-alkyl,
or R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
   which are selected independently from a group consisting of phenyl and naphthyl, and
   which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an aryl-heteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cyc1, in which Cyc1 is a ring system of formula Ia

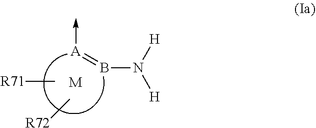

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,

and the salts of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and particularly the ethyl and methyl radicals.

2-4C-Alkyl represents a straight-chain or branched alkyl radical having 2 to 4 carbon atoms. Examples which may be mentioned are the butyl, isobutyl, sec-butyl, tert-butyl, propyl, isopropyl and particularly the ethyl radicals.

3-7C-Cycloalkyl stands for cyclopropyl, cyclobutyl, cyclopentyl, cyclohexyl and cycloheptyl, of which cyclopropyl, cyclobutyl and cyclopentyl are particular examples.

3-7C-Cycloalkylmethyl stands for a methyl radical, which is substituted by one of the abovementioned 3-7C-cycloalkyl radicals. Particular examples which may be mentioned are the cyclopropylmethyl, the cyclobutylmethyl and the cyclopentylmethyl radicals.

1-4C-Alkylene is a branched or, particularly, straight chain alkylene radical having 1 to 4 carbon atoms. Examples which may be mentioned are the methylene (—CH₂—), ethylene (dimethylene) (—CH₂—CH₂—), trimethylene (—CH₂—CH₂—CH₂—) and the tetramethylene (—CH₂—CH₂—CH₂—CH₂—) radical.

2-4C-Alkylene is a branched or, particularly, straight chain alkylene radical having 2 to 4 carbon atoms. Examples which may be mentioned are the ethylene (dimethylene) (—CH₂—CH₂—), trimethylene (—CH₂—CH₂—CH₂—) and the tetramethylene (—CH₂—CH₂—CH₂—CH₂—) radical.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and particularly the ethoxy and methoxy radicals.

1-4C-Alkoxy-1-4C-alkyl stands for one of the abovementioned 1-4C-alkyl radicals, which is substituted by one of the abovementioned 1-4C-alkoxy radicals. Examples which may be mentioned are the methoxymethyl, 2-methoxyethyl, 3-methoxypropyl and the 2-ethoxyethyl radical.

1-4C-Alkoxy-2-4C-alkyl stands for one of the abovementioned 2-4C-alkyl radicals, which is substituted by one of the abovementioned 1-4C-alkoxy radicals. Examples which may be mentioned are the 2-methoxyethyl, 3-methoxypropyl and the 2-ethoxyethyl radical.

Hydroxy-1-4C-alkyl stands for one of the abovementioned 1-4C-alkyl radicals which is substituted by hydroxyl. Examples which may be mentioned are the hydroxymethyl radical, the 2-hydroxyethyl radical or the 3-hydroxypropyl radical.

Hydroxy-2-4C-alkyl stands for one of the abovementioned 2-4C-alkyl radicals which is substituted by hydroxyl. Examples which may be mentioned are the 2-hydroxyethyl radical or the 3-hydroxypropyl radical.

Phenyl-1-4C-alkyl represents one of the abovementioned 1-4C-alkyl radicals, which is substituted by a phenyl radical. Examples which may be mentioned are the benzyl and phenethyl radicals.

Mono- or Di-1-4C-alkylamino radicals contain in addition to the nitrogen atom, one or two of the abovementioned 1-4C-alkyl radicals. Particular examples red are the di-1-4C-alkylamino radicals, especially the dimethylamino, the diethylamino and the diisopropylamino radical.

Mono- or Di-1-4C-alkylaminocarbonyl radicals contain in addition to the carbonyl group one of the abovementioned mono- or di-1-4C-alkylamino radicals. Examples which may be mentioned are the N-methyl- the N,N-dimethyl-, the N-ethyl-, the N-propyl-, the N,N-diethyl- and the N-isopropylaminocarbonyl radical, of which the N,N-dimethylaminocarbonyl radical is a particular example.

Mono- or Di-1-4C-alkylaminosulphonyl stands for a sulphonyl group to which one of the abovementioned mono- or di-1-4C-alkylamino radicals is bonded. Examples which may be mentioned are the methylaminosulphonyl, the dimethylaminosulphonyl and the ethylaminosulphonyl radical, of which the N,N-dimethylaminosulphonyl (dimethylsulphamoyl) radical [(CH₃)₂NS(O)₂—] is a particular example.

An 1-4C-Alkylcarbonylamino radical is, for example, the propionylamino (C₂H₅C(O)NH—) and the acetylamino (acetamido) radical (CH₃C(O)NH—).

An 1-4C-Alkylsulphonylamino radical is, for example, the ethanesulphonylamino (ethylsulphonylamino) (C₂H₅S(O)₂NH—) and the methanesulphonylamino (methylsulphonylamino) radical (CH₃S(O)₂NH—).

1-4C-Alkylsulfonyl is a sulfonyl group to which one of the abovementioned 1-4C-alkyl radicals is bonded. An example is the methanesulphonyl (methylsulphonyl) radical (CH₃SO₂—).

1-4C-Alkylcarbonyl is a carbonyl group to which one of the abovementioned 1-4C-alkyl radicals is bonded. An example is the acetyl radical (CH₃CO—).

Tolyl alone or as part of another group includes o-tolyl, m-tolyl and p-tolyl.

Halogen within the meaning of the invention is bromine or, in particular, chlorine or fluorine.

Aa1 is a bisaryl radical made up of two aryl groups,

which are selected independently from a group consisting of phenyl and naphthyl, and

which are linked together via a single bond.

Aa1 may include, without being restricted thereto, the biphenyl radical, e.g. the 1,1′-biphenyl-4-yl or 1,1′-biphenyl-3-yl radical.

As non-limiting examples of R61-substituted derivatives of Aa1 may be mentioned the following radicals:

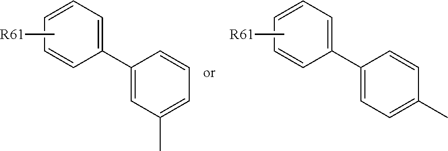

in which the substituent R61 can be attached in the ortho, or, in particular, meta or para position with respect to the binding position in which the benzene ring is bonded to the phenyl radical, such as e.g. 2′-(R61)-1,1′-biphenyl-3-yl, 2′-(R61)-1,1′-biphenyl-4-yl, or, in particular, 3′-(R61)-1,1′-biphenyl-3-yl or 3′-(R61)-1,1′-biphenyl-4-yl, or, yet in particular, 4′-(R61)-1,1′-biphenyl-3-yl or 4′-(R61)-1,1′-biphenyl-4-yl.

As exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is -T2-N(R611)R612, in which
T2 is methylene, dimethylene or trimethylene, and
R611 and R612 together and with inclusion of the nitrogen atom, to which they are bonded, form a morpholino or 4N-methyl-piperazino, or a piperidino or pyrrolidino radical; such as, for example, any selected from
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl and 4′-(4-methyl-piperazin-1-ylmethyl)-biphenyl-3-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which R61 is -T2-N(R611)R612, in which T2 is methylene, dimethylene or trimethylene, and R611 and R612 are both methyl;

such as, for example, any selected from
2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl and 3′-dimethylaminomethyl-biphenyl-3-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is -T2-N(R611)R612, in which
T2 is methylene, dimethylene or trimethylene, and
R611 is hydrogen, cyclopropyl, cyclopentyl, 2-methoxyethyl, acetyl or methylsulfonyl,
R612 is hydrogen;

for example,

either
R611 is cyclopropyl or 2-methoxyethyl, and
R612 is hydrogen,

such as, for example, any selected from
4′-(2-methoxy-ethylamino)methyl-biphenyl-3-yl and 4′-cyclopropylaminomethyl-biphenyl-3-yl,

or
R611 is hydrogen, cyclopentyl, acetyl or methylsulfonyl, and
R612 is hydrogen,

such as, for example, any selected from
4′-aminomethyl-biphenyl-3-yl, 4′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl and 4′-cyclopentylaminomethyl-biphenyl-4-yl.

Yet as exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is —O-T3-N(R613)R614, in which T3 is dimethylene or trimethylene, and
R613 and R614 together and with inclusion of the nitrogen atom, to which they are bonded, form a morpholino, pyrrolidino or 4N-methyl-piperazino, or a piperidino radical;

such as, for example, any selected from
4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 3′-(3-pyrrolidin-1-yl-propoxy]-biphenyl-4-yl, 4′-(3-pyrrolidin-1-yl-propoxy]-biphenyl-4-yl, 3′-(2-pyrrolidin-1-yl-ethoxy]-biphenyl-4-yl, 4′-(3-morpholin-4-yl-propoxy)-biphenyl-4-yl, 3′-(3-morpholin-4-yl-propoxy)-biphenyl-4-yl, 3′-(2-morpholin-4-yl-ethoxy)-biphenyl-4-yl, 4′-(2-morpholin-4-yl-ethoxy)-biphenyl-4-yl, 4′-[2-(4-methyl-piperazin-1-yl)-ethoxy]-biphenyl-4-yl, 4′-[3-(4-methyl-piperazin-1-yl)-propoxy]-biphenyl-4-yl and 3′-[3-(4-methyl-piperazin-1-yl)-propoxy]-biphenyl-4-yl.

Yet as exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is —O-T5-Het4, in which T5 is a bond, methylene, dimethylene or trimethylene, and
Het4 is 1-methyl-piperidin-4-yl;

such as e.g. 4′-(2-(1-methyl-piperidin-4-yl)-ethoxy)-biphenyl-4-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is methylsulphonylamino, N,N-dimethylaminosulphonyl, acetamido, hydroxymethyl, amino, dimethylamino, morpholino, hydroxyl, trifluoromethyl or methoxy;

for example,

either
R61 is methylsulphonylamino, N,N-dimethylaminosulphonyl, acetamido or hydroxymethyl, such as, for example, any selected from 2′-methylsulphonylamino-biphenyl-4-yl, 3′-methylsulphonylamino-biphenyl-4-yl, 4′-methylsulphonylamino-biphenyl-4-yl, 4′-methylsulphonylamino-biphenyl-3-yl, 4′-dimethylsulphamoyl-biphenyl-4-yl, 3′-acetamido-biphenyl-4-yl, 4′-acetamido-biphenyl-4-yl and 3′-hydroxymethyl-biphenyl-4-yl,

or
R61 is amino, dimethylamino, morpholino, hydroxyl, trifluoromethyl or methoxy, such as, for example, any selected from 3′-amino-biphenyl-4-yl, 4′-morpholin-4-yl-biphenyl-4-yl, 4′-hydroxy-biphenyl-4-yl, 3′-trifluoromethyl-biphenyl-4-yl, 3′-dimethylamino-biphenyl-4-yl and 4′-methoxy-biphenyl-4-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which R61 is —C(O)—N(H)-T3-N(R613)R614, in which T3 is dimethylene or trimethylene, and
R613 and R614 are both methyl;

such as, for example, any selected from
3′-[(2-dimethylamino-ethylamino)-carbonyl]-biphenyl-4-yl, 4′-[(2-dimethylamino-ethylamino)-carbonyl]-biphenyl-4-yl and 4′-[(2-dimethylamino-ethylamino)-carbonyl]-biphenyl-3-yl.

An example of R61-substituted Aa1 radicals may be 3′-(R61)-1,1′-biphenyl-3-yl, in which R61 is any one selected from the group G_{Aa1} consisting of 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl.

Another example of R61-substituted Aa1 radicals may be 3′-(R61)-1,1′-biphenyl-4-yl, in which R61 is any one selected from the group G_{Aa1} given above.

Another example of R61-substituted Aa1 radicals may be 4′-(R61)-1,1′-biphenyl-3-yl, in which R61 is any one selected from the group G_{Aa1} given above.

Another example of R61-substituted Aa1 radicals may be 4′-(R61)-1,1′-biphenyl-4-yl, in which R61 is any one selected from the group G_{Aa1} given above.

Specifically, as an exemplary R61-substituted Aa1 radical may be explicitly mentioned, for example, any one selected from 3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl, 4′-(4-methyl-piperazin-1-ylmethyl)-biphenyl-3-yl, 4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 3′-(3-pyrrolidin-1-yl-propoxy]-biphenyl-4-yl, 4′-(3-pyrrolidin-1-yl-propoxy]-biphenyl-4-yl, 3′-(2-pyrrolidin-1-yl-ethoxy]-biphenyl-4-yl, 4′-(3-morpholin-4-yl-propoxy)-biphenyl-4-yl, 3′-(3-morpholin-4-yl-propoxy)-biphenyl-4-yl, 3′-(2-morpholin-4-yl-ethoxy)-biphenyl-4-yl, 4′-(2-morpholin-4-yl-ethoxy)-biphenyl-4-yl, 4′-[2-(4-methyl-piperazin-1-yl)-ethoxy]-biphenyl-4-yl, 4′-[3-(4-methyl-piperazin-1-yl)-propoxy]-biphenyl-4-yl, 3′-[3-(4-methyl-piperazin-1-yl)-propoxy]-biphenyl-4-yl, 4′-(2-(1-methyl-piperidin-4-yl)-ethoxy)-biphenyl-4-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 4′-methylsulphonylamino-biphenyl-3-yl, 4′-dimethylsulphamoyl-biphenyl-4-yl, 3′-acetamido-biphenyl-4-yl, 4′-acetamido-biphenyl-4-yl, 3′-amino-biphenyl-4-yl, 4′-morpholin-4-yl-biphenyl-4-yl, 4′-hydroxy-biphenyl-4-yl, 3′-trifluoromethyl-biphenyl-4-yl and 4′-methoxy-biphenyl-4-yl, 4′-(2-methoxy-ethylamino)methyl-biphenyl-3-yl, 4′-aminomethyl-biphenyl-3-yl, 4′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-cyclopentylaminomethyl-biphenyl-4-yl, 4′-cyclopropylaminomethyl-biphenyl-3-yl, and 3′-hydroxymethyl-biphenyl-4-yl.

More specifically, as an exemplary R61-substituted Aa1 radical may be more explicitly mentioned, for example, any one selected from 4′-(2-morpholin-4-yl-ethyl)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, and 4′-dimethylaminomethyl-biphenyl-4-yl.

Hh1 is a bisheteroaryl radical made up of two heteroaryl groups,

which are selected independently from a group consisting of monocyclic 5- or 6-membered heteroaryl radicals comprising one or two heteroatoms, each of which is selected from the group consisting of nitrogen, oxygen and sulfur, and which are linked together via a single bond.

Hh1 may include, without being restricted thereto, the bithiophenyl e.g. thiophen-3-yl-thiophenyl or thiophen-2-yl-thiophenyl, bipyridyl, pyrazolyl-pyridinyl e.g. pyrazol-1-yl-pyridinyl or pyrazol-4-yl-pyridinyl like 6-(pyrazol-4-yl)-pyridin-3-yl, imidazolyl-pyridinyl e.g. imidazol-1-yl-pyridinyl, pyrazolyl-thiophenyl e.g. pyrazol-4-yl-thiophenyl like 5-(pyrazol-4-yl)-thiophen-2-yl, or pyridinyl-thiophenyl radical e.g. pyridin-2-yl-thiophenyl, pyridin-3-yl-thiophenyl or pyridin-4-yl-thiophenyl like 5-(pyridin-2-yl)-thiophen-2-yl or 5-(pyridin-4-yl)-thiophen-2-yl, or the thiazolyl-thiophenyl e.g. thiazol-4-yl-thiophenyl like 5-(thiazol-4-yl)-thiophen-2-yl, or thiazolyl-pyridinyl radical like 6-(thiazol-4-yl)-pyridin-3-yl.

In a special detail, exemplary Hh1 radicals may include pyridinyl-thiophenyl, e.g. 5-(pyridin-4-yl)-thiophen-2-yl. In another special detail, exemplary Hh1 radicals may include pyrazolyl-thiophenyl, e.g. 5-(pyrazol-4-yl)-thiophen-2-yl. In another special detail, exemplary Hh1 radicals may include bipyridyl, e.g. 2,4′-bipyridyl-5-yl. In another special detail, exemplary Hh1 radicals may include thiazolyl-thiophenyl, e.g. 5-(thiazol-4-yl)-thiophen-2-yl. In another special detail, exemplary Hh1 radicals may include pyrazolyl-pyridinyl, e.g. 6-(pyrazol-4-yl)-pyridin-3-yl. In another special detail, exemplary Hh1 radicals may include thiazolyl-pyridinyl, e.g. 6-(thiazol-4-yl)-pyridin-3-yl.

As non-limiting example of R61-substituted derivatives of Hh1 may be mentioned [1N-(1-4C-alkyl)-pyrazolyl]-thiophenyl, such as e.g. [1N-(1-4C-alkyl)-pyrazol-4-yl]-thiophenyl, like 5-[1N-(1-2C-alkyl)-pyrazol-4-yl]-thiophen-2-yl, e.g. 5-(1N-methyl-pyrazol-4-yl)-thiophen-2-yl.

Yet as non-limiting example of R61-substituted derivatives of Hh1 may be mentioned [1N-(1-4C-alkyl)-pyrazolyl]-pyridinyl, such as e.g. [1N-(1-4C-alkyl)-pyrazol-4-yl]-pyridinyl or 6-[1N-(1-4C-alkyl)-pyrazolyl]-pyridin-3-yl, like 6-[1N-(1-2C-alkyl)-pyrazol-4-yl]-pyridin-3-yl, e.g. 6-(1N-methyl-pyrazol-4-yl)-pyridin-3-yl.

Yet as non-limiting example of R61-substituted derivatives of Hh1 may be mentioned [(R61)-pyridinyl]-thiophenyl, such as e.g. the following radicals:

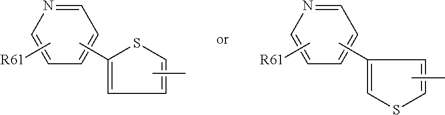

in which the substituent R61 can be attached in the ortho, or, in particular, meta or para position with respect to the binding position in which the pyridinyl ring is bonded to the thiophenyl radical, such as e.g. [2-(R61)-pyridin-4-yl]-thiophenyl or [6-(R61)-pyridin-3-yl]-thiophenyl, like 5-[2-(R61)-pyridin-4-yl]-thiophen-2-yl or 5-[6-(R61)-pyridin-3-yl]-thiophen-2-yl.

Yet as non-limiting example of R61-substituted derivatives of Hh1 may be mentioned [(R61)-thiazolyl]-thiophenyl, such as e.g. the following radicals:

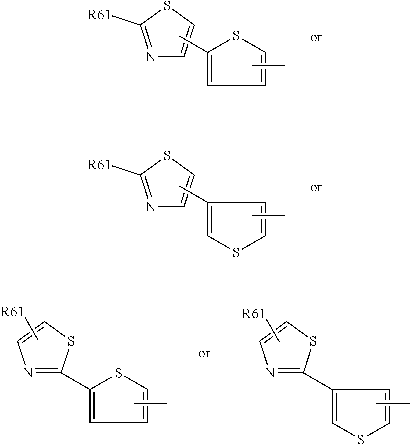

such as e.g. [2-(R61)-thiazol-4-yl]-thiophenyl, like 5-[2-(R61)-thiazol-4-yl]-thiophen-2-yl.

Yet as non-limiting example of R61-substituted derivatives of Hh1 may be mentioned [(R61)-pyridinyl]-pyridinyl, such as e.g. the following radicals:

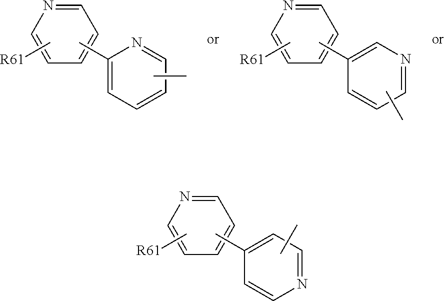

in which the substituent R61 can be attached in the ortho, or, in particular, meta or para position with respect to the binding position in which the terminal pyridinyl ring is bonded to the other pyridinyl radical, such as e.g. [2-(R61)-pyridin-4-yl]-pyridinyl or [6-(R61)-pyridin-3-yl]-pyridinyl or 6-[(R61)-pyridinyl]-pyridin-3-yl, like 6-[2-(R61)-pyridin-4-yl]-pyridin-3-yl [i.e. 2′-(R61)-2,4′-bipyridyl-5-yl] or 6-[6-(R61)-pyridin-3-yl]-pyridin-3-yl [i.e. 6′-(R61)-2,3′-bipyridyl-5-yl].

As exemplary R61-substituted Hh1 radicals may be more detailed mentioned, for example, 5-[2-(R61)-pyridin-4-yl]-thiophen-2-yl or 5-[6-(R61)-pyridin-3-yl]-thiophen-2-yl, in which
R61 is -T2-N(R611)R612, in which T2 is a bond, and
R611 and R612 are both hydrogen, or
R611 and R612 together and with inclusion of the nitrogen atom, to which they are bonded, form a morpholino or 4N-methyl-piperazino, or a piperidino or pyrrolidino radical; such as e.g. 5-[2-(4-methyl-piperazin-1-yl)-pyridin-4-yl]-thiophen-2-yl.

Yet as exemplary R61-substituted Hh1 radicals may be more detailed mentioned, for example, 2′-(R61)-2,4′-bipyridyl-5-yl or 6′-(R61)-2,3′-bipyridyl-5-yl, in which
R61 is -T2-N(R611)R612, in which T2 is a bond, and
R611 and R612 are both hydrogen, or
R611 and R612 together and with inclusion of the nitrogen atom, to which they are bonded, form a morpholino, 4N-methyl-piperazino, piperidino or pyrrolidino radical; such as e.g. 2′-(4-methyl-piperazin-1-yl)-2,4′-bipyridyl-5-yl.

Specifically, as an exemplary R61-substituted Hh1 radical may be explicitly mentioned, for example, any one selected from 5-[2-(4-methyl-piperazin-1-yl)-pyridin-4-yl]-thiophen-2-yl, 5-(1N-methyl-pyrazol-4-yl)-thiophen-2-yl, 2′-(4-methyl-piperazin-1-yl)-2,4′-bipyridyl-5-yl, 5-(2-methyl-thiazol-4-yl)-thiophen-2-yl, and 6-(1N-methyl-pyrazol-4-yl)-pyridin-3-yl.

More specifically, as an exemplary R61-substituted Hh1 radical may be more explicitly mentioned, for example, 5-[2-(4-methyl-piperazin-1-yl)-pyridin-4-yl]-thiophen-2-yl.

Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group.

Ah1 may include, without being restricted thereto, the phenyl-thiophenyl e.g. 5-phenyl-thiophen-2-yl, or the phenyl-pyridyl e.g. 6-phenyl-pyridin-3-yl, radical.

In a special detail, exemplary Ah1 radicals may include phenyl-thiophenyl, e.g. 5-(phenyl)-thiophen-2-yl.

Yet in a special detail, exemplary Ah1 radicals may include phenyl-pyridinyl, e.g. 6-(phenyl)-pyridin-3-yl.

As non-limiting example of R61-substituted derivatives of Ah1 may be mentioned [(R61)-phenyl]-thiophenyl, such as e.g. the following radicals:

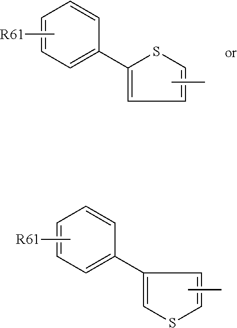

in which the substituent R61 can be attached in the ortho, or, in particular, meta or para position with respect to the binding position in which the phenyl ring is bonded to the thiophenyl radical, such as e.g. [3-(R61)-phenyl]-thiophenyl or [4-(R61)-phenyl]-thiophenyl, like 5-[3-(R61)-phenyl]-thiophen-2-yl or 5-[4-(R61)-phenyl]-thiophen-2-yl.

Yet as non-limiting example of R61-substituted derivatives of Ah1 may be mentioned [(R61)-phenyl]-pyridinyl, such as e.g. the following radicals:

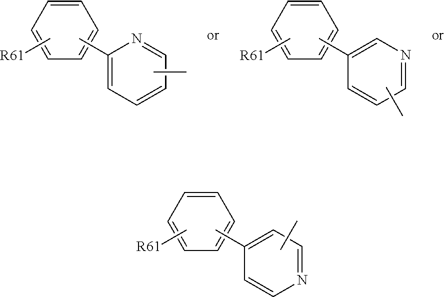

in which the substituent R61 can be attached in the ortho, or, in particular, meta or para position with respect to the binding position in which the phenyl ring is bonded to the pyridinyl radical, such as e.g. [3-(R61)-phenyl]-pyridinyl or [4-(R61)-phenyl]-pyridinyl or 6-[(R61)-phenyl]-pyridin-3-yl, like 6-[3-(R61)-phenyl]-pyridin-3-yl or 6-[4-(R61)-phenyl]-pyridin-3-yl.

As exemplary R61-substituted Ah1 radicals may be more detailed mentioned, for example, 5-[3-(R61)-phenyl]-thiophen-2-yl or 5-[4-(R61)-phenyl]-thiophen-2-yl, in which R61 is -T2-N(R611)R612, in which T2 is methylene, dimethylene or trimethylene, and R611 and R612 together and with inclusion of the nitrogen atom, to which they are bonded, form a morpholino or 4N-methyl-piperazino, or a piperidino or pyrrolidino radical; such as, for example, any selected from 5-[4-(2-morpholin-4-yl-ethyl)-phenyl]-thiophen-2-yl, 5-[4-(morpholin-4-yl-methyl)-phenyl]-thiophen-2-yl and 5-[3-(morpholin-4-yl-methyl)-phenyl]-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which R61 is -T2-N(R611)R612, in which T2 is methylene, dimethylene or trimethylene, and R611 and R612 are both methyl;

such as, for example, any selected from 5-(4-dimethylaminomethyl-phenyl)-thiophen-2-yl and 5-(3-dimethylaminomethyl-phenyl)-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which R61 is -T2-N(R611)R612, in which T2 is methylene, dimethylene or trimethylene, and R611 is hydrogen, cyclopropyl, cyclopentyl, 2-methoxyethyl, acetyl or methylsulfonyl, R612 is hydrogen;

such as, for example, any selected from 5-(3-aminomethyl-phenyl)-thiophen-2-yl, 5-[3-(acetylamino)-methyl-phenyl]-thiophen-2-yl and 5-[3-(methylsulphonylamino)-methyl-phenyl]-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which
R61 is methylsulphonylamino, N,N-dimethylaminosulphonyl, acetamido, hydroxymethyl, amino, dimethylamino, morpholino, hydroxyl, trifluoromethyl or methoxy;

such as e.g. 5-(4-dimethylsulphamoyl-phenyl)-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which R61 is —O-T3-N(R613)R614, in which T3 is dimethylene or trimethylene, and R613 and R614 together and with inclusion of the nitrogen atom, to which they are bonded, form a morpholino, pyrrolidino or 4N-methyl-piperazino, or a piperidino radical; such as, for example, any selected from 5-[4-(2-morpholin-4-yl-ethoxy)-phenyl]-thiophen-2-yl, 5-[4-(3-morpholin-4-yl-propoxy)-phenyl]-thiophen-2-yl, 5-{4-[2-(4-methyl-piperazin-1-yl)-ethoxy]-phenyl}-thiophen-2-yl and 5-[4-(2-pyrrolidin-1-yl-ethoxy)-phenyl]-thiophen-2-yl.

Yet as exemplary R61-substituted Ah1 radicals may be more detailed mentioned, for example, 6-[3-(R61)-phenyl]-pyridin-3-yl or 6-[4-(R61)-phenyl]-pyridin-3-yl, in which R61 is -T2-N(R611)R612, in which T2 is methylene, dimethylene or trimethylene, and R611 and R612 are both methyl;

such as, for example, any selected from
6-(4-dimethylaminomethyl-phenyl)-pyridin-3-yl and 6-(3-dimethylaminomethyl-phenyl)-pyridin-3-yl.

Yet as exemplary R61-substituted Ah1 radicals may be more detailed mentioned, for example, 6-[3-(R61)-phenyl]-pyridin-3-yl or 6-[4-(R61)-phenyl]-pyridin-3-yl, in which R61 is —O-T3-N(R613)R614, in which T3 is dimethylene or trimethylene, and
R613 and R614 together and with inclusion of the nitrogen atom, to which they are bonded, form a morpholino, piperidino, pyrrolidino or 4N-methyl-piperazino radical; such as e.g. 6-[4-(2-pyrrolidin-1-yl-ethoxy)-phenyl]-pyridin-3-yl.

An example of R61-substituted Ah1 radicals may be [4-(R61)-phenyl]-pyridinyl, e.g. 6-[4-(R61)-phenyl]-pyridin-3-yl, in which R61 is any one selected from the group G_{Ah1} consisting of 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl.

Another example of R61-substituted Ah1 radicals may be [3-(R61)-phenyl]-pyridinyl, e.g. 6-[3-(R61)-phenyl]-pyridin-3-yl, in which R61 is any one selected from the group G_{Ah1} given above. A further example of R61-substituted Ah1 radicals may be [4-(R61)-phenyl]-thiophenyl, e.g. 5-[4-(R61)-phenyl]-thiophen-2-yl, in which R61 is any one selected from the group G_{Ah1} given above. Another example of R61-substituted Ah1 radicals may be [3-(R61)-phenyl]-thiophenyl, e.g. 5-[3-(R61)-phenyl]-thiophen-2-yl, in which R61 is any one selected from the group G_{Ah1} given above.

Specifically, as an exemplary R61-substituted Ah1 radical may be explicitly mentioned, for example, any one selected from 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 5-[4-(2-morpholin-4-yl-ethoxy)-phenyl]-thiophen-2-yl, 5-[4-(3-morpholin-4-yl-propoxy)-phenyl]-thiophen-2-yl, 5-{4-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl, 6-(4-dimethylaminomethyl-phenyl)-pyridin-3-yl, 6-(3-dimethylaminomethyl-phenyl)-pyridin-3-yl, and 6-[4-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, and 5-(4-dimethylsulphamoyl-phenyl)-thiophen-2-yl.

More specifically, as an exemplary R61-substituted Ah1 radical may be more explicitly mentioned, for example, 5-(4-dimethylaminomethyl-phenyl)-thiophen-2-yl.

It is to be stated, that each of the radicals Hh1 and Ah1 is bonded via a ring carbon atom to the moiety T1.

Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group.

A particular embodiment of said Ha1 radicals refers to heteroaryl-phenyl radicals, particularly 3-(heteroaryl)-phenyl or 4-(heteroaryl)-phenyl radicals.

Ha1 may include, without being restricted thereto, the furanyl-phenyl, thiophenyl-phenyl, pyrazolyl-phenyl e.g. pyrazol-1-yl-phenyl or pyrazol-4-yl-phenyl, imidazolyl-phenyl e.g. imidazol-1-yl-phenyl, isoxazolyl-phenyl, or pyridinyl-phenyl radicals, or the thiazolyl-phenyl e.g. thiazol-4-yl-phenyl radical.

In a special detail, exemplary Ha1 radicals may include pyrazolyl-phenyl, e.g. 3-(pyrazolyl)-phenyl or 4-(pyrazolyl)-phenyl. Yet in a special detail, exemplary Ha1 radicals may include pyridinyl-phenyl, e.g. 4-(pyridinyl)-phenyl or 3-(pyridinyl)-phenyl. Yet in a special detail, exemplary Ha1 radicals may include isoxazolyl-phenyl, e.g. 4-(isoxazolyl)-phenyl or 3-(isoxazolyl)-phenyl. Yet in a special detail, exemplary Ha1 radicals may include thiazolyl-phenyl, e.g. 4-(thiazolyl)-phenyl or 3-(thiazolyl)-phenyl.

In a further special detail, exemplary Ha1 radicals may include 3-(pyrazol-1-yl)-phenyl, 4-(pyrazol-1-yl)-phenyl, 4-(pyridin-4-yl)-phenyl, 3-(pyridin-4-yl)-phenyl, 4-(pyridin-3-yl)-phenyl, 3-(pyridin-3-yl)-phenyl, 4-(isoxazol-4-yl)-phenyl, 3-(isoxazol-4-yl)-phenyl, 3-(pyrazol-4-yl)-phenyl or 4-(pyrazol-4-yl)-phenyl.

As non-limiting example of R61-substituted derivatives of Ha1 may be mentioned [1N-(1-4C-alkyl)-pyrazolyl]-phenyl, such as e.g. [1N-(1-4C-alkyl)-pyrazol-4-yl]-phenyl, like 3-[1N-(1-2C-alkyl)-pyrazol-4-yl]-phenyl or 4-[1N-(1-2C-alkyl)-pyrazol-4-yl]-phenyl, e.g. 3-(1N-methyl-pyrazol-4-yl)-phenyl or 4-(1N-methyl-pyrazol-4-yl)-phenyl.

As non-limiting example of R61- and/or R62-substituted derivatives of Ha1 may be mentioned (methyl-isoxazolyl)-phenyl or (dimethyl-isoxazolyl)-phenyl, such as e.g. 3-(3,5-dimethyl-isoxazol-4-yl)-phenyl or 4-(3,5-dimethyl-isoxazol-4-yl)-phenyl.

Yet as non-limiting example of R61-substituted derivatives of Ha1 may be mentioned [(R61)-pyridinyl]-phenyl, such as e.g. the following radicals:

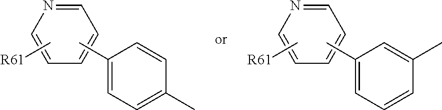

in which the substituent R61 can be attached in the ortho, or, in particular, meta or para position with respect to the binding position in which the pyridinyl ring is bonded to the phenyl radical, such as e.g. 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl.

As exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is -T2-N(R611)R612, in which T2 is a bond, and
R611 and R612 together and with inclusion of the nitrogen atom, to which they are bonded, form a morpholino or 4N-methyl-piperazino, or a piperidino or pyrrolidino radical;

such as, for example, any selected from
4-[2-(4-methyl-piperazin-1-yl)-pyridin-4-yl]-phenyl and 3-[2-(4-methyl-piperazin-1-yl)-pyridin-4-yl]-phenyl.

Yet as exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is -T2-N(R611)R612, in which T2 is a bond, and
R611 and R612 are both hydrogen;

such as, for example, any selected from 4-[6-amino-pyridin-3-yl]-phenyl and 3-[6-amino-pyridin-3-yl]-phenyl.

Yet as exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which R61 is methoxy; such as, for example, any selected from 4-[6-methoxy-pyridin-3-yl]-phenyl and 3-[6-methoxy-pyridin-3-yl]-phenyl.

Specifically, as an exemplary R61-substituted Ha1 radical may be explicitly mentioned, for example, any one selected from 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, 3-[6-amino-pyridin-3-yl]-phenyl, 4-[6-methoxy-pyridin-3-yl]-phenyl, 3-[6-methoxy-pyridin-3-yl]-phenyl, 3-(1N-methyl-pyrazol-4-yl)-phenyl, 4-(1N-methyl-pyrazol-4-yl)-phenyl, and 4-(3,5-dimethyl-isoxazol-4-yl)-phenyl.

More specifically, as an exemplary R61-substituted Ha1 radical may be more explicitly mentioned, for example, any one selected from 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, and 4-(1N-methyl-pyrazol-4-yl)-phenyl.

As part of the radicals Hh1, Ah1 and Ha1, the mentioned heteroaryl group selected from a group consisting of monocyclic 5- or 6-membered heteroaryl radicals comprising one or two heteroatoms, each of which is selected from the group consisting of nitrogen, oxygen and sulphur, may be chosen, for example, from the group consisting of, the 5-membered heteroaryl radicals, pyrrolyl, furanyl, thiophenyl, oxazolyl, isoxazolyl, thiazolyl, isothiazolyl, imidazolyl and pyrazolyl, and, the 6-membered heteroaryl radicals, pyridinyl, pyrimidinyl, pyrazinyl and pyridazinyl.

Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the to the parent molecular group.

A particular embodiment of said Ha2 radicals refers to heteroaryl-phenyl radicals, particularly 3-(heteroaryl)-phenyl or 4-(heteroaryl)-phenyl radicals.

Another particular embodiment of said Ha2 radicals refers to heteroaryl-phenyl radicals, particularly 3-(heteroaryl)-phenyl or 4-(heteroaryl)-phenyl radicals, in which the heteroaryl moiety contains a benzene ring.

Another particular embodiment of said Ha2 radicals refers to heteroaryl-phenyl radicals, particularly 3-(heteroaryl)-phenyl or 4-(heteroaryl)-phenyl radicals, in which the heteroaryl moiety contains a benzene ring, and whereby the heteroaryl moiety is attached via said benzene ring to the phenyl moiety.

Ha2 may include, without being restricted thereto, the indolyl-phenyl, benzothiophenyl-phenyl, benzofuranyl-phenyl, benzoxazolyl-phenyl, benzothiazolyl-phenyl, indazolyl-phenyl, benzimidazolyl-phenyl, benzisoxazolyl-phenyl, benzisothiazolyl-phenyl, benzofurazanyl-phenyl, benzotriazolyl-phenyl, benzothiadiazolyl-phenyl, quinolinyl-phenyl, isoquinolinyl-phenyl, quinazolinyl-phenyl, quinoxalinyl-phenyl, cinnolinyl-phenyl, indolizinyl-phenyl or naphthyridinyl-phenyl.

In a special detail, exemplary Ha2 radicals may include 3-(indolyl)-phenyl or 4-(indolyl)-phenyl.

In a further special detail, exemplary Ha2 radicals may include 3-(indol-5-yl)-phenyl or 4-(indol-5-yl)-phenyl.

Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group, A particular embodiment of said Ha3 radicals refers to heteroaryl-phenyl radicals, particularly 3-(heteroaryl)-phenyl or 4-(heteroaryl)-phenyl radicals.

Ha3 may include, without being restricted thereto, the thiadiazolyl-phenyl (e.g. [1,3,4]thiadiazol-2-yl-phenyl or [1,2,5]thiadiazol-3-yl-phenyl), oxadiazolyl-phenyl (e.g. [1,3,4]oxadiazol-2-yl-phenyl or [1,2,4]oxadiazol-5-yl-phenyl), triazolyl-phenyl (e.g. triazol-1-yl-phenyl or [1,2,3]triazol-4-yl) or tetrazolyl-phenyl (e.g. tetrazol-1-yl-phenyl or tetrazol-5-yl-phenyl) radicals.

In a special detail, exemplary Ha3 radicals may include triazolyl-phenyl, e.g. 3-(triazolyl)-phenyl or 4-(triazolyl)-phenyl. In a further special detail, exemplary Ha3 radicals may include 3-[1,2,3]triazol-4-yl-phenyl or 4-[1,2,3]triazol-4-yl-phenyl.

As non-limiting example of R61-substituted derivatives of Ha3 may be mentioned {1N—(R61)-[1,2,3]triazolyl}-phenyl, such as e.g. {1N—(R61)-[1,2,3]triazol-4-yl}-phenyl, like 3-{1N—(R61)-[1,2,3]triazol-4-yl}-phenyl or 4-{1N—(R61)-[1,2,3]triazol-4-yl}-phenyl.

As exemplary R61-substituted Ha3 radicals may be more detailed mentioned, for example, 3-[1N—(R61)-1,2,3-triazol-4-yl]-phenyl or 4-{1N—(R61)-[1,2,3]triazol-4-yl}-phenyl, in which R61 is -T2-N(R611)R612, in which T2 is dimethylene or trimethylene, and R611 and R612 together and with inclusion of the nitrogen atom, to which they are bonded, form a piperidino, pyrrolidino, morpholino or 4N-methyl-piperazino radical; such as e.g. 4-{1-(2-morpholin-4-yl-ethyl)-[1,2,3]triazol-4-yl}-phenyl or 4-{1-(2-piperidin-1-yl-ethyl)-[1,2,3]triazol-4-yl}-phenyl.

Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group, A particular embodiment of said Ha4 radicals refers to heteroaryl-phenyl radicals, particularly 3-(heteroaryl)-phenyl or 4-(heteroaryl)-phenyl radicals.

Another particular embodiment of said Ha4 radicals refers to heteroaryl-phenyl radicals, particularly 3-(heteroaryl)-phenyl or 4-(heteroaryl)-phenyl radicals, whereby the heteroaryl moiety is attached via its benzene ring to the phenyl moiety.

Ha4 may include, without being restricted thereto, the indolinyl-phenyl, isoindolinyl-phenyl, (1,2,3,4-tetrahydroquinolinyl)-phenyl or (1,2,3,4-tetrahydroisoquinolinyl)-phenyl, (2,3-dihydrobenzofuranyl)-phenyl, (2,3-dihydrobenzothiophenyl)-phenyl, (benzo[1,3]dioxolyl)-phenyl, (2,3-dihydrobenzo[1,4]dioxinyl)-phenyl, chromanyl-phenyl, chromenyl-phenyl or (2,3-dihydrobenzo[1,4]oxazinyl)-phenyl.

In a special detail, exemplary Ha4 radicals may include (benzo[1,3]dioxolyl)-phenyl, e.g. 3-(benzo[1,3]dioxolyl)-phenyl or 4-(benzo[1,3]dioxolyl)-phenyl, such as, for example, (benzo[1,3]dioxol-5-yl)-phenyl, e.g. 3-(benzo[1,3]dioxol-5-yl)-phenyl or 4-(benzo[1,3]dioxol-5-yl)-phenyl. Yet in a special detail, exemplary Ha4 radicals may include (2,3-dihydrobenzofuranyl)-phenyl, e.g. 3-(2,3-dihydrobenzofuranyl)-phenyl or 4-(2,3-dihydrobenzofuranyl)-phenyl, such as, for example, (2,3-dihydrobenzofuran-5-yl)-phenyl or (2,3-dihydrobenzofuran-6-yl)-phenyl, e.g. 3-(2,3-dihydrobenzofuran-5-yl)-phenyl or 4-(2,3-dihydrobenzofuran-5-yl)-phenyl. In a further special detail, exemplary Ha4 radicals may include 4-(2,3-dihydrobenzofuran-5-yl)-phenyl.

Har2 stands for a monocyclic 5- or 6-membered unsaturated heteroaromatic ring comprising one to three heteroatoms, each of which is selected from the group consisting of nitrogen, oxygen and sulfur.

Har2 may include, without being restricted thereto, thiophene, oxazole, isoxazole, thiazole, isothiazole, imidazole, pyrazole, triazole, thiadiazole, oxadiazole, pyridine, pyrimidine, pyrazine or pyridazine.

In a special detail, an exemplary Har2 radical may be pyridine.

Cyc1 stands for a ring system of formula Ia, which is bonded to the nitrogen atom of the carboxamide group via the moiety A. Cyc1 may include, without being restricted thereto, 2-aminophenyl substituted by R71 and/or R72. In a special detail, an exemplary Cyc1 radical may be 2-aminophenyl.

Naphthyl, alone or as part of another group, includes naphthalen-1-yl and naphthalen-2-yl.

In the meaning of the present invention, it is to be understood, that, when two structural portions of the compounds according to this invention are linked via a constituent which has the meaning “bond”, then said two portions are directly attached to another via a single bond.

When R61 has the meaning of —U-T3-N(R613)R614, in which U stands for —C(O)NH—, then R61 is the radical —C(O)NH-T3-N(R613)R614.

As it is known for the skilled person, the expressions morpholino, 4N-(1-4C-alkyl)-piperazino, pyrrolidino and the like stand for morpholin-4-yl, 4N-(1-4C-alkyl)-piperazin-1-yl, pyrrolidin-1-yl and the like, respectively.

In general, unless otherwise mentioned the heterocyclic groups mentioned herein refer to all of the possible isomeric forms thereof. The heterocyclic groups mentioned herein refer, unless otherwise noted, in particular to all of the possible positional isomers thereof. Thus, for example, the term pyridyl or pyridinyl, alone or as part of another group, includes pyridin-2-yl, pyridin-3-yl and pyridin-4-yl.

Constituents which are optionally substituted as stated herein, may be substituted, unless otherwise noted, at any possible position.

The carbocyclic groups, alone or as part of other groups, mentioned herein may be substituted by their given substituents or parent molecular groups, unless otherwise noted, at any substitutable ring carbon atom.

The heterocyclic groups, alone or as part of other groups, mentioned herein may be substituted by their given substituents or parent molecular groups, unless otherwise noted, at any possible position, such as e.g. at any substitutable ring carbon or ring nitrogen atom.

Rings containing quaternizable imino-type ring nitrogen atoms (—N═) may be particularly not quaternized on these imino-type ring nitrogen atoms by the mentioned substituents or parent molecular groups.

Any heteroatom of a heterocyclic ring with unsatisfied valences mentioned herein is assumed to have the hydrogen atom(s) to satisfy the valences.

When any variable occurs more than one time in any constituent, each definition is independent.

According to expert's knowledge the compounds of formula I of the invention as well as their salts may contain, e.g. when isolated in crystalline form, varying amounts of solvents.

Included within the scope of the invention are therefore all solvates and in particular all hydrates of the compounds of formula I as well as all solvates and in particular all hydrates of the salts of the compounds of formula I.

The substituents R61 and R62 of compounds of formula I can be attached in any possible position of the Aa1, Hh1, Ha1, Ha2, Ha3, Ha4 or Ah1 radical, whereby emphasis is given to the attachment at the terminal ring;

in another embodiment, Q1 is monosubstituted by R61, and is Aa1, Hh1, Ha1 or Ah1, whereby emphasis is given to the attachment of R61 at the terminal ring;

in yet another embodiment, R6 is Aa1, Ha1 or Ha2, each of which is monosubstituted by R61, whereby emphasis is given to the attachment of R61 at the terminal ring;

in yet another embodiment, R6 is Aa1, Hh1, Ha1, Ha2 or Ah1, each of which is monosubstituted by R61, whereby emphasis is given to the attachment of R61 at the terminal ring;

in yet another embodiment, R6 is Aa1, Hh1, Ha1, Ha2, Ha3 or Ah1, each of which is monosubstituted by R61, whereby emphasis is given to the attachment of R61 at the terminal ring;

in yet another embodiment, R6 is Ha2, Ha3 or Ha4, each of which is unsubstituted.

Within the meaning of this invention, the terminal ring of Aa1, Hh1, Ha1, Ha2, Ha3, Ha4 or Ah1 refers to those ring portion of these radicals which is not directly attached to the T1 moiety.

The person skilled in the art is aware on account of his/her expert knowledge that certain combinations of the variable characteristics mentioned in the description of this invention may lead to chemically less stable compounds. This can apply, for example, to certain compounds, in which—in a manner being disadvantageous for chemical stability—two heteroatoms (S, N or O) would directly meet or would only be separated by one carbon atom. Particularly, the compounds according to this invention are those, in which the combination of the abovementioned variable substituents does not lead to chemically less stable compounds.

Compounds according to aspect A of the present invention more worthy to be mentioned are those compounds of formula I in which
R1, R2, R3, R4 and R5 are independently hydrogen or 1-4C-alkyl,
R6 is -T1-Q1, in which T1 is a bond,

either
Q1 is substituted by R61 and/or R62, and is Aa1, Hh1, Ha1, Ha2, Ha3, Ha4 or Ah1,
Or Q1 is unsubstituted, and is Ha2, Ha3 or Ha4,

in which
R61 is 1-4C-alkyl, 1-4C-alkoxy, hydroxyl, trifluoromethyl, halogen, hydroxy-1-4C-alkyl, 1-4C-alkylsulphonylamino, tolylsulphonylamino, phenylsulphonylamino, 1-4C-alkylcarbonylamino, di-1-4C-alkylaminosulphonyl, -T2-N(R611)R612, —U-T3-N(R613)R614, -T4-Het3, or —V-T5-Het4, in which T2 is a bond or 1-4C-alkylene,
R611 is hydrogen, 1-4C-alkyl, 3-7C-cycloalkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 1-4C-alkoxy-2-4C-alkyl, 1-4C-alkylcarbonyl, or 1-4C-alkylsulphonyl,
R614 is hydrogen or 1-4C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond, or 1-4C-alkylene,
Het4 is 1N-(1-4C-alkyl)-piperidinyl or 1N-(1-4C-alkyl)-pyrrolidinyl,
R62 is 1-4C-alkyl,
Aa1 is biphenyl,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
Ha4 is a heteroaryl-phenyl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and a phenyl group, whereby said heteroaryl and phenyl groups are linked together via a single bond, and whereby Ha4 is bonded via said phenyl moiety to the to the parent molecular group,
R7 is hydroxyl, or 2-aminophenyl,

and the salts of these compounds.

Compounds according to aspect A of the present invention in particular worthy to be mentioned are those compounds of formula I in which
R1, R2, R3, R4 and R5 are hydrogen,
R6 is -T1-Q1, in which T1 is a bond,

either
Q1 is substituted by R61 and/or R62 on the terminal ring, and is Aa1, Hh1, Ha1, Ha2, Ha3, Ha4 or Ah1,
Or Q1 is unsubstituted, and is Ha2, Ha3 or Ha4,

in which
R61 is 1-2C-alkyl, 1-2C-alkoxy, hydroxyl, trifluoromethyl, halogen, hydroxy-1-2C-alkyl, 1-2C-alkylsulphonylamino, 1-2C-alkylcarbonylamino, di-1-2C-alkylaminosulphonyl, -T2-N(R611)R612, —U-T3-N(R613)R614, -T4-Het3, or —V-T5-Het4, in which
T2 is a bond or straight chain 1-4C-alkylene,
R611 is hydrogen, 1-2C-alkyl, 3-5C-cycloalkyl, 1-2C-alkoxy-2-3C-alkyl, 1-2C-alkylcarbonyl, or 1-2C-alkylsulphonyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl, 1-2C-alkoxy-2-3C-alkyl, 1-2C-alkylcarbonyl, or 1-2C-alkylsulphonyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-2C-alkyl)-piperazino,
T4 is a bond or straight chain 1-4C-alkylene,
Het3 is 1N-(1-2C-alkyl)-piperidinyl or 1N-(1-2C-alkyl)-pyrrolidinyl,
V is —O— (oxygen) or —C(O)NH—,
T5 is a bond or straight chain 1-4C-alkylene,
Het4 is 1N-(1-2C-alkyl)-piperidinyl or 1N-(1-2C-alkyl)-pyrrolidinyl,
R62 is 1-2C-alkyl,
Aa1 is 1,1′-biphenyl-3-yl or 1,1′-biphenyl-4-yl,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a 3-(heteroaryl)-phenyl or 4-(heteroaryl)-phenyl radical each made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
Ha4 is a 3-(heteroaryl)-phenyl or 4-(heteroaryl)-phenyl radical each made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and a phenyl group, whereby said heteroaryl and phenyl groups are linked together via a single bond, and whereby Ha4 is bonded via said phenyl moiety to the to the parent molecular group,
R7 is hydroxyl, or 2-aminophenyl,

and the salts of these compounds.

Compounds according to aspect A of the present invention in more particular worthy to be mentioned are those compounds of formula I in which
R1, R2, R3, R4 and R5 are hydrogen,
R6 is -T1-Q1, in which T1 is a bond,

either
Q1 is substituted by R61 and/or R62 on the terminal ring, and is Aa1, Hh1, Ha1, Ha2,
Ha3, Ha4 or Ah1,
or Q1 is
   unsubstituted, and is Ha2, Ha3 or Ha4,

in which
R61 is methyl, methoxy, hydroxyl, trifluoromethyl, hydroxymethyl, methylsulphonylamino, methylcarbonylamino, dimethylaminosulphonyl, -T2-N(R611)R612, —U-T3-N(R613)R614, -T4-Het3, or —V-T5-Het4, in which
T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4-methyl-piperazino,
U is —O— (oxygen) or —C(O)NH—,
T3 is dimethylene or trimethylene,
R613 is hydrogen, methyl, cyclopropyl, cyclopentyl, 2-methoxyethyl, acetyl or methylsulphonyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl,
R62 is methyl,
Aa1 is 1,1′-biphenyl-3-yl, or 1,1′-biphenyl-4-yl,
Hh1 is a bisheteroaryl radical made up of two heteroaryl groups, which are selected independently from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, and
   which are linked together via a single bond,

such as, for example,
Hh1 is pyridinyl-thiophenyl, thiazolyl-thiophenyl, pyrazolyl-thiophenyl, bipyridyl, pyrazolyl-pyridinyl, or thiazolyl-pyridinyl,
Ah1 is a phenyl-heteroaryl radical made up of an phenyl group and a heteroaryl group selected from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, whereby said phenyl and heteroaryl groups are linked together via a single bond, and whereby Ah1 is bonded via said heteroaryl moiety to the parent molecular group,

such as, for example,
Ah1 is phenyl-thiophenyl, or phenyl-pyridinyl,
Ha1 is a 3-(heteroaryl)-phenyl or 4-(heteroaryl)-phenyl radical each made up of a heteroaryl group selected from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, and a phenyl group, whereby said heteroaryl and phenyl groups are linked together via a single bond, and whereby Ha1 is bonded via said phenyl moiety to the to the parent molecular group,

such as, for example,
Ha1 is 3-(pyridinyl)-phenyl, 3-(thiazolyl)-phenyl, 3-(pyrazolyl)-phenyl, 3-(isoxazolyl)-phenyl, 4-(pyridinyl)-phenyl, 4-(thiazolyl)-phenyl, 4-(pyrazolyl)-phenyl, or 4-(isoxazolyl)-phenyl,
Ha2 is a 3-(heteroaryl)-phenyl or 4-(heteroaryl)-phenyl radical each made up of a heteroaryl group selected from a group consisting of indolyl, benzothiophenyl, benzofuranyl, benzoxazolyl, benzothiazolyl, indazolyl, benzimidazolyl, benzisoxazolyl, benzisothiazolyl, benzofurazanyl, benzotriazolyl, benzothiadiazolyl, quinolinyl, isoquinolinyl, quinazolinyl, quinoxalinyl, cinnolinyl, indolizinyl and naphthyridinyl, and a phenyl group, whereby said heteroaryl and phenyl groups are linked together via a single bond, and whereby Ha2 is bonded via said phenyl moiety to the to the parent molecular group,

such as, for example,
Ha2 is 3-(indolyl)-phenyl, or 4-(indolyl)-phenyl,
Ha3 is a 3-(heteroaryl)-phenyl or 4-(heteroaryl)-phenyl radical each made up of a heteroaryl group selected from a group consisting of thiadiazolyl, oxadiazolyl, triazolyl and tetrazolyl, and a phenyl group, whereby said heteroaryl and phenyl groups are linked together via a single bond, and whereby Ha3 is bonded via said phenyl moiety to the to the parent molecular group,

such as, for example,
Ha3 is 3-(triazolyl)-phenyl, or 4-(triazolyl)-phenyl,
Ha4 is a 3-(heteroaryl)-phenyl or 4-(heteroaryl)-phenyl radical each made up of a heteroaryl group selected from a group consisting of indolinyl, isoindolinyl, 1,2,3,4-tetrahydroquinolinyl, 1,2,3,4-tetrahydroisoquinolinyl, 2,3-dihydrobenzofuranyl, 2,3-dihydrobenzothiophenyl, benzo[1,3]dioxolyl, 2,3-dihydrobenzo[1,4]dioxinyl, chromanyl, chromenyl and 2,3-dihydrobenzo[1,4]oxazinyl, and a phenyl group, whereby said heteroaryl and phenyl groups are linked together via a single bond, and whereby Ha3 is bonded via said phenyl moiety to the to the parent molecular group,

such as, for example,
Ha4 is 3-(benzo[1,3]dioxolyl)-phenyl, 4-(benzo[1,3]dioxolyl)-phenyl, 3-(2,3-dihydrobenzofuranyl)-phenyl, or 4-(2,3-dihydrobenzofuranyl)-phenyl,
R7 is hydroxyl, or 2-aminophenyl, and the salts of these compounds.

Compounds according to aspect A of the present invention to be emphasized are those compounds of formula I in which
R1, R2, R3, R4 and R5 are hydrogen,
R6 is -T1-Q1, in which T1 is a bond;

either
Q1 is substituted by R61 on the terminal ring, and is Aa1 or Ah1, in which
Aa1 is 1,1′-biphenyl-3-yl, or 1,1′-biphenyl-4-yl,

such as, for example,
3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,

such as, for example,
[3-(R61)-phenyl]-thiophenyl, [4-(R61)-phenyl]-thiophenyl, [3-(R61)-phenyl]-pyridinyl or [4-(R61)-phenyl]-pyridinyl,
e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,

in which
R61 is methoxy, hydroxyl, trifluoromethyl, hydroxymethyl, methylsulphonylamino, methylcarbonylamino, dimethylaminosulphonyl, -T2-N(R611)R612, —U-T3-N(R613)R614, -T4-Het3, or —V-T5-Het4, in which
T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
U is —O— (oxygen) or —C(O)NH—,
T3 is dimethylene or trimethylene,
R613 and R614 are methyl,
or R613 and R614 together and with inclusion of the nitrogen atom, to which they are bonded, form a heterocyclic ring Het2, in which Het2 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl;

or
Q1 is substituted by R61 on the terminal ring, and is Hh1 or Ha1, in which Hh1 is pyridinyl-thiophenyl, or bipyridyl,

such as, for example,
[2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl,

or
[2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,

such as, for example,
3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,

in which
R61 is methoxy, or -T2-N(R611)R612, in which
T2 is a bond,
R611 and R612 are independently hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino;

or
Q1 is 3-(1-methyl-pyrazolyl)-phenyl, 4-(1-methyl-pyrazolyl)-phenyl, 3-(methyl-thiazolyl)-phenyl, 4-(methyl-thiazolyl)-phenyl, 3-(dimethyl-isoxazolyl)-phenyl, 4-(dimethyl-isoxazolyl)-phenyl, (1-methyl-pyrazolyl)-thiophenyl, (1-methyl-pyrazolyl)-pyridinyl, (methyl-thiazolyl)-thiophenyl,
   (methyl-thiazolyl)-pyridinyl, 3-(benzo[1,3]dioxolyl)-phenyl, 4-(benzo[1,3]dioxolyl)-phenyl, 3-(2,3-dihydrobenzofuranyl)-phenyl, 4-(2,3-dihydrobenzofuranyl)-phenyl, 3-(1-methyl-indolyl)-phenyl, or 4-(1-methyl-indolyl)-phenyl,

such as, for example,
3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl or 4-(1-methyl-indol-5-yl)-phenyl;

or
Q1 is 3-[1N—(R61)-pyrazolyl]-phenyl, 4-[1N—(R61)-pyrazolyl]-phenyl, [1N—(R61)-pyrazolyl)-thiophenyl, [1N—(R61)-pyrazolyl)-pyridinyl, 3-[1N—(R61)-triazolyl]-phenyl, or 4-[1N—(R61)-triazolyl]-phenyl,

such as, for example,
3-[1N—(R61)-pyrazol-4-yl]-phenyl, 4-[1N—(R61)-pyrazol-4-yl]-phenyl, [1N—(R61)-pyrazol-4-yl)-thiophenyl e.g. 5-[1N—(R61)-pyrazol-4-yl)-thiophen-2-yl, [1N—(R61)-pyrazol-4-yl)-pyridinyl e.g. 2-[1N—(R61)-pyrazol-4-yl)-pyridin-4-yl or 6-[1N—(R61)-pyrazol-4-yl)-pyridin-3-yl, 3-[1N—(R61)-triazol-4-yl]-phenyl or 4-[1N—(R61)-triazol-4-yl]-phenyl,

in which
R61 is -T2-N(R611)R612, or -T4-Het3, in which
T2 is dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl;
R7 is hydroxyl;

and the salts of these compounds.

Other compounds according to aspect A of the present invention to be emphasized are those compounds of formula I in which
R1, R2, R3, R4 and R5 are hydrogen,
R6 is -T1-Q1, in which
T1 is a bond;

either
Q1 is substituted by R61 on the terminal ring, and is Aa1 or Ah1, in which
Aa1 is 1,1′-biphenyl-3-yl, or 1,1′-biphenyl-4-yl,

such as, for example, 3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,

such as, for example,
[3-(R61)-phenyl]-thiophenyl, [4-(R61)-phenyl]-thiophenyl, [3-(R61)-phenyl]-pyridinyl or [4-(R61)-phenyl]-pyridinyl,
e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,

in which
R61 is methoxy, hydroxyl, trifluoromethyl, hydroxymethyl, methylsulphonylamino, methylcarbonylamino, dimethylaminosulphonyl, -T2-N(R611)R612, —U-T3-N(R613)R614, -T4-Het3, or —V-T5-Het4, in which
T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
U is —O— (oxygen) or —C(O)NH—,
T3 is dimethylene or trimethylene,
R613 is methyl,
R614 is methyl,
or R613 and R614 together and with inclusion of the nitrogen atom, to which they are bonded, form a heterocyclic ring Het2, in which
Het2 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl;

or
Q1 is substituted by R61 on the terminal ring, and is Hh1 or Ha1, in which Hh1 is pyridinyl-thiophenyl, or bipyridyl,

such as, for example,
[2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl,

or
[2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,

such as, for example,
3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,

in which
R61 is methoxy, or -T2-N(R611)R612, in which
T2 is a bond,
R611 is hydrogen or methyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino;

or
Q1 is 3-(1-methyl-pyrazolyl)-phenyl, 4-(1-methyl-pyrazolyl)-phenyl, 3-(methyl-thiazolyl)-phenyl, 4-(methyl-thiazolyl)-phenyl, 3-(dimethyl-isoxazolyl)-phenyl, 4-(dimethyl-isoxazolyl)-phenyl,
   (1-methyl-pyrazolyl)-thiophenyl, (1-methyl-pyrazolyl)-pyridinyl, (methyl-thiazolyl)-thiophenyl,
   (methyl-thiazolyl)-pyridinyl, 3-(benzo[1,3]dioxolyl)-phenyl, 4-(benzo[1,3]dioxolyl)-phenyl, 3-(2,3-dihydrobenzofuranyl)-phenyl, 4-(2,3-dihydrobenzofuranyl)-phenyl, 3-(1-methyl-indolyl)-phenyl, or 4-(1-methyl-indolyl)-phenyl,

such as, for example,
3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl or 4-(1-methyl-indol-5-yl)-phenyl;

or
Q1 is 3-[1N—(R61)-pyrazolyl]-phenyl, 4-[1N—(R61)-pyrazolyl]-phenyl, [1N—(R61)-pyrazolyl)-thiophenyl, [1N—(R61)-pyrazolyl)-pyridinyl, 3-[1N—(R61)-triazolyl]-phenyl, or 4-[1N—(R61)-triazolyl]-phenyl,

such as, for example,
3-[1N—(R61)-pyrazol-4-yl]-phenyl, 4-[1N—(R61)-pyrazol-4-yl]-phenyl, [1N—(R61)-pyrazol-4-yl)-thiophenyl e.g. 5-[1N—(R61)-pyrazol-4-yl)-thiophen-2-yl, [1N—(R61)-pyrazol-4-yl)-pyridinyl e.g. 2-[1N—(R61)-pyrazol-4-yl)-pyridin-4-yl or 6-[1N—(R61)-pyrazol-4-yl)-pyridin-3-yl,
3-[1N—(R61)-triazol-4-yl]-phenyl or 4-[1N—(R61)-triazol-4-yl]-phenyl,

in which
R61 is -T2-N(R611)R612, or -T4-Het3, in which
T2 is dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl;
R7 is 2-aminophenyl;

and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond;

either
Q1 is substituted by R61 on the terminal ring, and is Aa1 or Ah1, in which
Aa1 is 1,1′-biphenyl-3-yl, or 1,1′-biphenyl-4-yl,

such as, for example, 3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,

such as, for example,
[3-(R61)-phenyl]-thiophenyl, [4-(R61)-phenyl]-thiophenyl, [3-(R61)-phenyl]-pyridinyl or [4-(R61)-phenyl]-pyridinyl,
e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,

in which
R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl;

or
Q1 is substituted by R61 on the terminal ring, and is Hh1 or Ha1, in which
Hh1 is pyridinyl-thiophenyl, or bipyridyl,

such as, for example,
[2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl,

or [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,

such as, for example,
3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,

in which
R61 is any one selected from methylsulphonylamino, acetamido, amino, dimethylamino, morpholino, piperidino, pyrrolidino, 4-methyl-piperazino, hydroxy, trifluoromethyl and methoxy;

or
Q1 is 3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl, or 4-(1-methyl-indol-5-yl)-phenyl;

or
Q1 is 3-[1N—(R61)-pyrazol-4-yl]-phenyl, 4-[1N—(R61)-pyrazol-4-yl]-phenyl, [1N—(R61)-pyrazol-4-yl)-thiophenyl e.g. 5-[1N—(R61)-pyrazol-4-yl)-thiophen-2-yl, [1N—(R61)-pyrazol-4-yl)-pyridinyl e.g. 2-[1N—(R61)-pyrazol-4-yl)-pyridin-4-yl or 6-[1N—(R61)-pyrazol-4-yl)-pyridin-3-yl, 3-[1N—(R61)-triazol-4-yl]-phenyl, or 4-[1N—(R61)-triazol-4-yl]-phenyl,

in which
R61 is any one selected from 3-morpholin-4-yl-propyl, 2-morpholin-4-yl-ethyl, 3-(4-methyl-piperazin-1-yl)-propyl, 2-(4-methyl-piperazin-1-yl)-ethyl, 3-pyrrolidin-1-yl-propyl, 2-pyrrolidin-1-yl-ethyl, 3-piperidin-1-yl-propyl, 2-piperidin-1-yl-ethyl, 2-dimethylamino-ethyl and 3-dimethylamino-propyl;
R7 is hydroxyl;

and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond;

either
Q1 is substituted by R61 on the terminal ring, and is Aa1 or Ah1, in which
Aa1 is 1,1′-biphenyl-3-yl, or 1,1′-biphenyl-4-yl,

such as, for example,
3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,

such as, for example,
[3-(R61)-phenyl]-thiophenyl, [4-(R61)-phenyl]-thiophenyl, [3-(R61)-phenyl]-pyridinyl or [4-(R61)-phenyl]-pyridinyl,
e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,

in which
R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl;

or
Q1 is substituted by R61 on the terminal ring, and is Hh1 or Ha1, in which Hh1 is pyridinyl-thiophenyl, or bipyridyl,

such as, for example,
[2-(R61)-pyridin-4-yl]-thiophenyl or [6-(R61)-pyridin-3-yl]-thiophenyl,
e.g. 5-[2-(R61)-pyridin-4-yl]-thiophen-2-yl or 5-[6-(R61)-pyridin-3-yl]-thiophen-2-yl,

or
[2-(R61)-pyridin-4-yl]-pyridinyl or [6-(R61)-pyridin-3-yl]-pyridinyl,
e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,

such as, for example, 3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,

in which
R61 is any one selected from methylsulphonylamino, acetamido, amino, dimethylamino, morpholino, piperidino, pyrrolidino, 4-methyl-piperazino, hydroxy, trifluoromethyl and methoxy;

or
Q1 is 3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl, or 4-(1-methyl-indol-5-yl)-phenyl;

or
Q1 is 3-[1N—(R61)-pyrazol-4-yl]-phenyl, 4-[1N—(R61)-pyrazol-4-yl]-phenyl, [1N—(R61)-pyrazol-4-yl)-thiophenyl e.g. 5-[1N—(R61)-pyrazol-4-yl)-thiophen-2-yl, [1N—(R61)-pyrazol-4-yl)-pyridinyl e.g. 2-[1N—(R61)-pyrazol-4-yl)-pyridin-4-yl or 6-[1N—(R61)-pyrazol-4-yl)-pyridin-3-yl, 3-[1N—(R61)-triazol-4-yl]-phenyl, or 4-[1N—(R61)-triazol-4-yl]-phenyl,

in which
R61 is any one selected from 3-morpholin-4-yl-propyl, 2-morpholin-4-yl-ethyl, 3-(4-methyl-piperazin-1-yl)-propyl, 2-(4-methyl-piperazin-1-yl)-ethyl, 3-pyrrolidin-1-yl-propyl, 2-pyrrolidin-1-yl-ethyl, 3-piperidin-1-yl-propyl, 2-piperidin-1-yl-ethyl, 2-dimethylamino-ethyl and 3-dimethylamino-propyl;
R7 is 2-aminophenyl;

and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond;
Q1 is any one selected from the group consisting of
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl, 4′-(3-morpholin-4-yl-propyl)-biphenyl-4-yl, 3′-(3-morpholin-4-yl-propyl)-biphenyl-3-yl, 3′-(3-morpholin-4-yl-propyl)-biphenyl-4-yl, 4′-(4-methyl-piperazin-1-ylmethyl)-biphenyl-3-yl, 4′-(4-methyl-piperazin-1-ylmethyl)-biphenyl-4-yl, 3′-(4-methyl-piperazin-1-ylmethyl)-biphenyl-3-yl, 3′-(4-methyl-piperazin-1-ylmethyl)-biphenyl-4-yl, 4′-(2-morpholin-4-yl-ethoxy)-biphenyl-3-yl, 4′-(2-morpholin-4-yl-ethoxy)-biphenyl-4-yl, 3′-(2-morpholin-4-yl-ethoxy)-biphenyl-3-yl, 3′-(2-morpholin-4-yl-ethoxy)-biphenyl-4-yl, 4′-(3-morpholin-4-yl-propoxy)-biphenyl-3-yl, 4′-(3-morpholin-4-yl-propoxy)-biphenyl-4-yl, 3′-(3-morpholin-4-yl-propoxy)-biphenyl-3-yl, 3′-(3-morpholin-4-yl-propoxy)-biphenyl-4-yl, 4′-[2-(4-methyl-piperazin-1-yl)-ethoxy]-biphenyl-3-yl, 4′-[2-(4-methyl-piperazin-1-yl)-ethoxy]-biphenyl-4-yl, 3′-[2-(4-methyl-piperazin-1-yl)-ethoxy]-biphenyl-3-yl, 3′-[2-(4-methyl-piperazin-1-yl)-ethoxy]-biphenyl-4-yl, 4′-(2-pyrrolidin-1-yl-ethoxy]-biphenyl-3-yl, 4′-(2-pyrrolidin-1-yl-ethoxy]-biphenyl-4-yl, 3′-(2-pyrrolidin-1-yl-ethoxy]-biphenyl-3-yl, 3′-(2-pyrrolidin-1-yl-ethoxy]-biphenyl-4-yl, 3′-(3-pyrrolidin-1-yl-propoxy]-biphenyl-4-yl, 4′-(3-pyrrolidin-1-yl-propoxy]-biphenyl-4-yl, 3′-(3-pyrrolidin-1-yl-propoxy]-biphenyl-3-yl, 4′-(3-pyrrolidin-1-yl-propoxy]-biphenyl-3-yl, 4′-[3-(4-methyl-piperazin-1-yl)-propoxy]-biphenyl-4-yl, 3′-[3-(4-methyl-piperazin-1-yl)-propoxy]-biphenyl-4-yl, 4′-[3-(4-methyl-piperazin-1-yl)-propoxy]-biphenyl-3-yl, 3′-[3-(4-methyl-piperazin-1-yl)-propoxy]-biphenyl-3-yl, 4′-(2-(1-methyl-piperidin-4-yl)-ethoxy)-biphenyl-4-yl, 4′-(2-(1-methyl-piperidin-4-yl)-ethoxy)-biphenyl-3-yl, 3′-(2-(1-methyl-piperidin-4-yl)-ethoxy)-biphenyl-4-yl, 3′-(2-(1-methyl-piperidin-4-yl)-ethoxy)-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 3′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 2′-methylsulphonylamino-biphenyl-3-yl, 3′-methylsulphonylamino-biphenyl-3-yl, 4′-methylsulphonylamino-biphenyl-3-yl, 4′-methylsulphonylamino-biphenyl-3-yl, 4′-dimethylsulphamoyl-biphenyl-4-yl, 4′-dimethylsulphamoyl-biphenyl-3-yl, 3′-dimethylsulphamoyl-biphenyl-4-yl, 3′-dimethylsulphamoyl-biphenyl-3-yl, 3′-acetamido-biphenyl-4-yl, 4′-acetamido-biphenyl-4-yl, 3′-acetamido-biphenyl-3-yl, 4′-acetamido-biphenyl-3-yl, 3′-amino-biphenyl-4-yl, 3′-dimethylamino-biphenyl-4-yl, 4′-morpholin-4-yl-biphenyl-4-yl, 4′-hydroxy-biphenyl-4-yl, 3′-trifluoromethyl-biphenyl-4-yl, 4′-methoxy-biphenyl-4-yl, 3′-amino-biphenyl-3-yl, 3′-dimethylamino-biphenyl-3-yl, 4′-morpholin-4-yl-biphenyl-3-yl, 4′-hydroxy-biphenyl-3-yl, 3′-trifluoromethyl-biphenyl-3-yl, 4′-methoxy-biphenyl-3-yl, 4′-amino-biphenyl-4-yl, 4′-dimethylamino-biphenyl-4-yl, 3′-morpholin-4-yl-biphenyl-4-yl, 3′-hydroxy-biphenyl-4-yl, 4′-trifluoromethyl-biphenyl-4-yl, 3′-methoxy-biphenyl-4-yl, 4′-amino-biphenyl-3-yl, 4′-dimethylamino-biphenyl-3-yl, 3′-morpholin-4-yl-biphenyl-3-yl, 3′-hydroxy-biphenyl-3-yl, 4′-trifluoromethyl-biphenyl-3-yl and 3′-methoxy-biphenyl-3-yl, 4′-(2-methoxy-ethylamino)methyl-biphenyl-3-yl, 4′-(2-methoxy-ethylamino)methyl-biphenyl-4-yl, 3′-(2-methoxy-ethylamino)methyl-biphenyl-3-yl, 3′-(2-methoxy-ethylamino)methyl-biphenyl-4-yl, 4′-aminomethyl-biphenyl-3-yl, 4′-aminomethyl-biphenyl-4-yl, 3′-aminomethyl-biphenyl-3-yl, 3′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-(acetylamino)-methyl-biphenyl-3-yl, 4′-(methylsulphonylamino)-methyl-biphenyl-3-yl, 3′-(acetylamino)-methyl-biphenyl-4-yl, 3′-(methylsulphonylamino)-methyl-biphenyl-4-yl, 4′-cyclopentylaminomethyl-biphenyl-4-yl, 4′-cyclopentylaminomethyl-biphenyl-3-yl, 3′-cyclopentylaminomethyl-biphenyl-4-yl, 3′-cyclopentylaminomethyl-biphenyl-3-yl, 4′-cyclopropylaminomethyl-biphenyl-3-yl, 4′-cyclopropylaminomethyl-biphenyl-4-yl, 3′-cyclopropylaminomethyl-biphenyl-3-yl, 3′-cyclopropylaminomethyl-biphenyl-4-yl, 3′-hydroxymethyl-biphenyl-4-yl, 3′-hydroxymethyl-biphenyl-3-yl, 4′-hydroxymethyl-biphenyl-4-yl, 4′-hydroxymethyl-biphenyl-3-yl, 5-[2-(4-methyl-piperazin-1-yl)-pyridin-4-yl]-thiophen-2-yl, 5-(1-methyl-pyrazol-4-yl)-thiophen-2-yl, 6-(1-methyl-pyrazol-4-yl)-pyridin-3-yl, 2′-(4-methyl-piperazin-1-yl)-2,4′-bipyridyl-5-yl, 5-(2-methyl-thiazol-4-yl)-thiophen-2-yl, 5-[4-(2-morpholin-4-yl-ethyl)-phenyl]-thiophen-2-yl, 5-[3-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 5-[4-(2-morpholin-4-yl-ethoxy)-phenyl]-thiophen-2-yl, 5-[3-(2-morpholin-4-yl-ethoxy)-phenyl]-thiophen-2-yl, 5-[4-(3-morpholin-4-yl-propoxy)-phenyl]-thiophen-2-yl, 5-[3-(3-morpholin-4-yl-propoxy)-phenyl]-thiophen-2-yl, 5-{4-[2-(4-methyl-piperazin-1-yl)-ethoxy]-phenyl}-thiophen-2-yl, 5-{3-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-[3-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl, 6-(4-dimethylaminomethyl-phenyl)-pyridin-3-yl, 6-(3-dimethylaminomethyl-phenyl)-pyridin-3-yl, 6-[4-(2-pyrrolidin-1-yl-ethoxy)-phenyl]-pyridin-3-yl, 6-[3-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, 5-(4-dimethylsulphamoyl-phenyl)-thiophen-2-yl, 5-(4-aminomethyl-phenyl)-thiophen-2-yl, 5-[4-(acetylamino)-methyl-phenyl]-thiophen-2-yl, 5-[4-(methylsulphonylamino)-methyl-phenyl]-thiophen-2-yl, 5-(3-dimethylsulphamoyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-(6-amino-pyridin-3-yl)-phenyl, 3-(6-amino-pyridin-3-yl)-phenyl, 4-(6-methoxy-pyridin-3-yl)-phenyl, 3-(6-methoxy-pyridin-3-yl)-phenyl, 3-(1-methyl-pyrazol-4-yl)-phenyl, 4-(1-methyl-pyrazol-4-yl)-phenyl, 4-(3,5-dimethyl-isoxazol-4-yl)-phenyl, 3-(3,5-dimethyl-isoxazol-4-yl)-phenyl, 4-(1-methyl-indol-5-yl)-phenyl, 3-(1-methyl-indol-5-yl)-phenyl, 4-{1-(2-morpholin-4-yl-ethyl)-[1,2,3]triazol-4-yl}-phenyl, 4-{1-(2-piperidin-1-yl-ethyl)-[1,2,3]triazol-4-yl}-phenyl, 3-{1-(2-morpholin-4-yl-ethyl)-[1,2,3]triazol-4-yl}-phenyl, 3-{1-(2-piperidin-1-yl-ethyl)-[1,2,3]triazol-4-yl}-phenyl, 4-(2,3-dihydrobenzofuran-5-yl)-phenyl, and 4-(benzo[1,3]dioxol-5-yl)-phenyl, 3-(2,3-dihydrobenzofuran-5-yl)-phenyl, and 3-(benzo[1,3]dioxol-5-yl)-phenyl,
R7 is hydroxyl,

and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond;
Q1 is any one selected from the group consisting of
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl, 4′-(3-morpholin-4-yl-propyl)-biphenyl-4-yl, 3′-(3-morpholin-4-yl-propyl)-biphenyl-3-yl, 3′-(3-morpholin-4-yl-propyl)-biphenyl-4-yl, 4′-(4-methyl-piperazin-1-ylmethyl)-biphenyl-3-yl, 4′-(4-methyl-piperazin-1-ylmethyl)-biphenyl-4-yl, 3′-(4-methyl-piperazin-1-ylmethyl)-biphenyl-3-yl, 3′-(4-methyl-piperazin-1-ylmethyl)-biphenyl-4-yl, 4′-(2-morpholin-4-yl-ethoxy)-biphenyl-3-yl, 4′-(2-morpholin-4-yl-ethoxy)-biphenyl-4-yl, 3′-(2-morpholin-4-yl-ethoxy)-biphenyl-3-yl, 3′-(2-morpholin-4-yl-ethoxy)-biphenyl-4-yl, 4′-(3-morpholin-4-yl-propoxy)-biphenyl-3-yl, 4′-(3-morpholin-4-yl-propoxy)-biphenyl-4-yl, 3′-(3-morpholin-4-yl-propoxy)-biphenyl-3-yl, 3′-(3-morpholin-4-yl-propoxy)-biphenyl-4-yl, 4′-[2-(4-methyl-piperazin-1-yl)-ethoxy]-biphenyl-3-yl, 4′-[2-(4-methyl-piperazin-1-yl)-ethoxy]-biphenyl-4-yl, 3′-[2-(4-methyl-piperazin-1-yl)-ethoxy]-biphenyl-3-yl, 3′-[2-(4-methyl-piperazin-1-yl)-ethoxy]-biphenyl-4-yl, 4′-(2-pyrrolidin-1-yl-ethoxy]-biphenyl-3-yl, 4′-(2-pyrrolidin-1-yl-ethoxy]-biphenyl-4-yl, 3′-(2-pyrrolidin-1-yl-ethoxy]-biphenyl-3-yl, 3′-(2-pyrrolidin-1-yl-ethoxy]-biphenyl-4-yl, 3′-(3-pyrrolidin-1-yl-propoxy]-biphenyl-4-yl, 4′-(3-pyrrolidin-1-yl-propoxy]-biphenyl-4-yl, 3′-(3-pyrrolidin-1-yl-propoxy]-biphenyl-3-yl, 4′-(3-pyrrolidin-1-yl-propoxy]-biphenyl-3-yl, 4′-[3-(4-methyl-piperazin-1-yl)-propoxy]-biphenyl-4-yl, 3′-[3-(4-methyl-piperazin-1-yl)-propoxy]-biphenyl-4-yl, 4′-[3-(4-methyl-piperazin-1-yl)-propoxy]-biphenyl-3-yl, 3′-[3-(4-methyl-piperazin-1-yl)-propoxy]-biphenyl-3-yl, 4′-(2-(1-methyl-piperidin-4-yl)-ethoxy)-biphenyl-4-yl, 4′-(2-(1-methyl-piperidin-4-yl)-ethoxy)-biphenyl-3-yl, 3′-(2-(1-methyl-piperidin-4-yl)-ethoxy)-biphenyl-4-yl, 3′-(2-(1-methyl-piperidin-4-yl)-ethoxy)-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 3′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 2′-methylsulphonylamino-biphenyl-3-yl, 3′-methylsulphonylamino-biphenyl-3-yl, 4′-methylsulphonylamino-biphenyl-3-yl, 4′-methylsulphonylamino-biphenyl-3-yl, 4′-dimethylsulphamoyl-biphenyl-4-yl, 4′-dimethylsulphamoyl-biphenyl-3-yl, 3′-dimethylsulphamoyl-biphenyl-4-yl, 3′-dimethylsulphamoyl-biphenyl-3-yl, 3′-acetamido-biphenyl-4-yl, 4′-acetamido-biphenyl-4-yl, 3′-acetamido-biphenyl-3-yl, 4′-acetamido-biphenyl-3-yl, 3′-amino-biphenyl-4-yl, 3′-dimethylamino-biphenyl-4-yl, 4′-morpholin-4-yl-biphenyl-4-yl, 4′-hydroxy-biphenyl-4-yl, 3′-trifluoromethyl-biphenyl-4-yl, 4′-methoxy-biphenyl-4-yl, 3′-amino-biphenyl-3-yl, 3′-dimethylamino-biphenyl-3-yl, 4′-morpholin-4-yl-biphenyl-3-yl, 4′-hydroxy-biphenyl-3-yl, 3′-trifluoromethyl-biphenyl-3-yl, 4′-methoxy-biphenyl-3-yl, 4′-amino-biphenyl-4-yl, 4′-dimethylamino-biphenyl-4-yl, 3′-morpholin-4-yl-biphenyl-4-yl, 3′-hydroxy-biphenyl-4-yl, 4′-trifluoromethyl-biphenyl-4-yl, 3′-methoxy-biphenyl-4-yl, 4′-amino-biphenyl-3-yl, 4′-dimethylamino-biphenyl-3-yl, 3′-morpholin-4-yl-biphenyl-3-yl, 3′-hydroxy-biphenyl-3-yl, 4′-trifluoromethyl-biphenyl-3-yl and 3′-methoxy-biphenyl-3-yl, 4′-(2-methoxy-ethylamino)methyl-biphenyl-3-yl, 4′-(2-methoxy-ethylamino)methyl-biphenyl-4-yl, 3′-(2-methoxy-ethylamino)methyl-biphenyl-3-yl, 3′-(2-methoxy-ethylamino)methyl-biphenyl-4-yl, 4′-aminomethyl-biphenyl-3-yl, 4′-aminomethyl-biphenyl-4-yl, 3′-aminomethyl-biphenyl-3-yl, 3′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-(acetylamino)-methyl-biphenyl-3-yl, 4′-(methylsulphonylamino)-methyl-biphenyl-3-yl, 3′-(acetylamino)-methyl-biphenyl-4-yl, 3′-(methylsulphonylamino)-methyl-biphenyl-4-yl, 4′-cyclopentylaminomethyl-biphenyl-4-yl, 4′-cyclopentylaminomethyl-biphenyl-3-yl, 3′-cyclopentylaminomethyl-biphenyl-4-yl, 3′-cyclopentylaminomethyl-biphenyl-3-yl, 4′-cyclopropylaminomethyl-biphenyl-3-yl, 4′-cyclopropylaminomethyl-biphenyl-4-yl, 3′-cyclopropylaminomethyl-biphenyl-3-yl, 3′-cyclopropylaminomethyl-biphenyl-4-yl, 3′-hydroxymethyl-biphenyl-4-yl, 3′-hydroxymethyl-biphenyl-3-yl, 4′-hydroxymethyl-biphenyl-4-yl, 4′-hydroxymethyl-biphenyl-3-yl, 5-[2-(4-methyl-piperazin-1-yl)-pyridin-4-yl]-thiophen-2-yl, 5-(1-methyl-pyrazol-4-yl)-thiophen-2-yl, 6-(1-methyl-pyrazol-4-yl)-pyridin-3-yl, 2′-(4-methyl-piperazin-1-yl)-2,4′-bipyridyl-5-yl, 5-(2-methyl-thiazol-4-yl)-thiophen-2-yl, 5-[4-(2-morpholin-4-yl-ethyl)-phenyl]-thiophen-2-yl, 5-[3-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 5-[4-(2-morpholin-4-yl-ethoxy)-phenyl]-thiophen-2-yl, 5-[3-(2-morpholin-4-yl-ethoxy)-phenyl]-thiophen-2-yl, 5-[4-(3-morpholin-4-yl-propoxy)-phenyl]-thiophen-2-yl, 5-[3-(3-morpholin-4-yl-propoxy)-phenyl]-thiophen-2-yl, 5-{4-[2-(4-methyl-piperazin-1-yl)-ethoxy]-phenyl}-thiophen-2-yl, 5-{3-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-[3-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl, 6-(4-dimethylaminomethyl-phenyl)-pyridin-3-yl, 6-(3-dimethylaminomethyl-phenyl)-pyridin-3-yl, 6-[4-(2-pyrrolidin-1-yl-ethoxy)-phenyl]-pyridin-3-yl, 6-[3-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, 5-(4-dimethylsulphamoyl-phenyl)-thiophen-2-yl, 5-(4-aminomethyl-phenyl)-thiophen-2-yl, 5-[4-(acetylamino)-methyl-phenyl]-thiophen-2-yl, 5-[4-(methylsulphonylamino)-methyl-phenyl]-thiophen-2-yl, 5-(3-dimethylsulphamoyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-(6-amino-pyridin-3-yl)-phenyl, 3-(6-amino-pyridin-3-yl)-phenyl, 4-(6-methoxy-pyridin-3-yl)-phenyl, 3-(6-methoxy-pyridin-3-yl)-phenyl, 3-(1-methyl-pyrazol-4-yl)-phenyl, 4-(1-methyl-pyrazol-4-yl)-phenyl, 4-(3,5-dimethyl-isoxazol-4-yl)-phenyl, 3-(3,5-dimethyl-isoxazol-4-yl)-phenyl, 4-(1-methyl-indol-5-yl)-phenyl, 3-(1-methyl-indol-5-yl)-phenyl, 4-{1-(2-morpholin-4-yl-ethyl)-[1,2,3]triazol-4-yl}-phenyl, 4-{1-(2-piperidin-1-yl-ethyl)-[1,2,3]triazol-4-yl}-phenyl, 3-{1-(2-morpholin-4-yl-ethyl)-[1,2,3]triazol-4-yl}-phenyl, 3-{1-(2-piperidin-1-yl-ethyl)-[1,2,3]triazol-4-yl}-phenyl, 4-(2,3-dihydrobenzofuran-5-yl)-phenyl, and 4-(benzo[1,3]dioxol-5-yl)-phenyl, 3-(2,3-dihydrobenzofuran-5-yl)-phenyl, and 3-(benzo[1,3]dioxol-5-yl)-phenyl,
R7 is 2-aminophenyl,

and the salts of these compounds.

Compounds according to aspect B of the present invention to be more emphasized are those compounds of formula I in which
R1, R2, R3, R4 and R5 are independently hydrogen or 1-4C-alkyl,
R6 is -T1-Q1, in which T1 is a bond;

either
Q1 is substituted by R61 and/or R62, and is Aa1, Hh1, Ha1, Ha2, Ha3 or Ah1,
Or Q1 is unsubstituted, and is Ha2 or Ha3,

in which
R61 is 1-4C-alkyl, 1-4C-alkoxy, halogen, hydroxy-1-4C-alkyl, 1-4C-alkylsulphonylamino, tolylsulphonylamino, phenylsulphonylamino, 1-4C-alkylcarbonylamino, di-1-4C-alkylaminosulphonyl, -T2-N(R611)R612, or —U-T3-N(R613)R614, in which
T2 is a bond or 1-4C-alkylene,
R611 is hydrogen, 1-4C-alkyl, 3-7C-cycloalkyl or 1-4C-alkoxy-2-4C-alkyl,
R612 is hydrogen or 1-4C-alkyl,
or R611 and R612 together and with inclusion of the nitrogen atom, to which they are bonded, form a heterocyclic ring Het1, in which Het1 is morpholino, piperidino, pyrrolidino, piperazino or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl or 1-4C-alkoxy-2-4C-alkyl,
R614 is hydrogen or 1-4C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-4C-alkyl,
Aa1 is biphenyl,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,

and the salts of these compounds.

Compounds according to aspect B of the present invention to be more emphasized are those compounds of formula I in which
R1, R2, R3, R4 and R5 are hydrogen,
R6 is -T1-Q1, in which T1 is a bond;
Q1 is substituted by R61 and/or R62 on the terminal ring, and is Aa1, Hh1, Ha1, Ha2 or Ah1,

in which
R61 is 1-2C-alkyl, 1-2C-alkoxy, halogen, hydroxy-1-2C-alkyl, 1-2C-alkylsulphonylamino, 1-2C-alkylcarbonylamino, di-1-2C-alkylaminosulphonyl, -T2-N(R611)R612, or —U-T3-N(R613)R614, in which
T2 is a bond or straight chain 1-4C-alkylene,
R611 is hydrogen, 1-2C-alkyl, 3-5C-cycloalkyl or 1-2C-alkoxy-2-3C-alkyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl or 1-2C-alkoxy-2-3C-alkyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-2C-alkyl,
Aa1 is 1,1′-biphenyl-3-yl or 1,1′-biphenyl-4-yl,
Hh1 is a bisheteroaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a thiophenyl group, whereby said heteroaryl and thiophenyl groups are linked together via a single bond, and whereby Hh1 is bonded via said thiophenyl moiety to the to the parent molecular group,
Ah1 is phenyl-thiophenyl,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
R7 is hydroxyl, or 2-aminophenyl,

and the salts of these compounds.

Compounds according to aspect B of the present invention to be more emphasized are those compounds of formula I in which
R1, R2, R3, R4 and R5 are hydrogen,
R6 is -T1-Q1, in which T1 is a bond;
Q1 is substituted by R61 and/or R62 on the terminal ring, and is Aa1, Hh1, Ha1, Ha2 or Ah1,

in which
R61 is 1-2C-alkyl, 1-2C-alkoxy, hydroxyl, trifluoromethyl, halogen, hydroxy-1-2C-alkyl, 1-2C-alkylsulphonylamino, 1-2C-alkylcarbonylamino, di-1-2C-alkylaminosulphonyl, -T2-N(R611)R612, or —U-T3-N(R613)R614, in which
T2 is a bond or straight chain 1-4C-alkylene,
R611 is hydrogen, 1-2C-alkyl, 3-5C-cycloalkyl or 1-2C-alkoxy-2-3C-alkyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl or 1-2C-alkoxy-2-3C-alkyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-2C-alkyl,
Aa1 is 1,1′-biphenyl-3-yl or 1,1′-biphenyl-4-yl,
Hh1 is a bisheteroaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a thiophenyl group, whereby said heteroaryl and thiophenyl groups are linked together via a single bond, and whereby Hh1 is bonded via said thiophenyl moiety to the to the parent molecular group,
Ah1 is phenyl-thiophenyl or phenyl-pyridinyl,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
R7 is hydroxyl, or 2-aminophenyl,

and the salts of these compounds.

Compounds according to aspect B of the present invention to be more emphasized are those compounds of formula I in which
R1, R2, R3, R4 and R5 are hydrogen,
R6 is -T1-Q1, in which T1 is a bond;
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl,
R61 is methoxy, hydroxymethyl, methylsulphonylamino, methylcarbonylamino, dimethylaminosulphonyl, -T2-N(R611)R612, or —U-T3-N(R613)R614, in which
T2 is a bond, methylene, dimethylene or trimethylene,
R611 is hydrogen, methyl, cyclopropyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino,
U is —O— (oxygen) or —C(O)NH—,
T3 is dimethylene or trimethylene,
R613 is hydrogen, methyl, cyclopropyl or 2-methoxyethyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4N-methyl-piperazino,

or
Q1 is substituted by R61 on the terminal ring, and is Hh1 or Ha1, in which
Hh1 is pyridinyl-thiophenyl,
Ha1 is 3-(pyridinyl)-phenyl or 4-(pyridinyl)-phenyl,
R61 is methoxy, or -T2-N(R611)R612, in which
T2 is a bond, methylene, dimethylene or trimethylene,
R611 is hydrogen, methyl, cyclopropyl or 2-methoxyethyl,
R612 is hydrogen or methyl,
or R611 and R612 together and with inclusion of the nitrogen atom, to which they are bonded, form a heterocyclic ring Het1, in which Het1 is morpholino, piperidino, pyrrolidino, piperazino or 4N-methyl-piperazino,

or
Q1 is 3-(1N-methyl-pyrazolyl)-phenyl, 4-(1N-methyl-pyrazolyl)-phenyl, 3-(1N-methyl-indolyl)-phenyl or 4-(1N-methyl-indolyl)-phenyl,
R7 is hydroxyl, or 2-aminophenyl,

and the salts of these compounds.

Yet compounds according to aspect B of the present invention in more particular worthy to be mentioned are those compounds of formula I in which
R1, R2, R3, R4 and R5 are all hydrogen,
R6 is -T1-Q1, in which T1 is a bond,
Q is (1N-methyl-pyrazolyl)-thiophenyl,
   3-(dimethyl-isoxazolyl)-phenyl or 4-(dimethyl-isoxazolyl)-phenyl,

and the salts of these compounds.

In another embodiment, still yet compounds according to aspect B of the present invention in more particular worthy to be mentioned are those compounds of formula I in which
R1, R2, R3, R4 and R5 are all hydrogen,
R6 is -T1-Q1, in which T1 is a bond,
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl or phenyl-pyridinyl,
R61 is methoxy, hydroxyl, trifluoromethyl, hydroxymethyl, methylsulphonylamino, methylcarbonylamino, dimethylaminosulphonyl, -T2-N(R611)R612, or —U-T3-N(R613)R614, in which
T2 is a bond, methylene, dimethylene or trimethylene,
R611 is hydrogen, methyl, cyclopropyl, cyclopentyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino,
U is —O— (oxygen) or —C(O)NH—,
T3 is dimethylene or trimethylene,
R613 is hydrogen, methyl, cyclopropyl, cyclopentyl or 2-methoxyethyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4N-methyl-piperazino,

or
Q1 is substituted by R61 on the terminal ring, and is Hh1 or Ha1, in which Hh1 is pyridinyl-thiophenyl or bipyridyl,
Ha1 is 3-(pyridinyl)-phenyl or 4-(pyridinyl)-phenyl,
R61 is methoxy, or -T2-N(R611)R612, in which
T2 is a bond, methylene, dimethylene or trimethylene,
R611 is hydrogen, methyl, cyclopropyl, cyclopentyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino,

or
Q1 is 3-(1N-methyl-pyrazolyl)-phenyl, 4-(1N-methyl-pyrazolyl)-phenyl, (1N-methyl-pyrazolyl)-thiophenyl, (1N-methyl-pyrazolyl)-pyridinyl, 3-(methyl-thiazolyl)-phenyl, 4-(methyl-thiazolyl)-phenyl, (methyl-thiazolyl)-thiophenyl, (methyl-thiazolyl)-pyridinyl, 3-(dimethyl-isoxazolyl)-phenyl, 4-(dimethyl-isoxazolyl)-phenyl, 3-(1N-methyl-indolyl)-phenyl or 4-(1N-methyl-indolyl)-phenyl,
R7 is hydroxyl, or 2-aminophenyl,

and the salts of these compounds.

Compounds according to aspect B of the present invention to be emphasized are those compounds of formula I in which
R1, R2, R3, R4 and R5 are all hydrogen,
R6 is -T1-Q1, in which T1 is a bond,
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl,
R61 is hydroxymethyl, methylsulphonylamino, methylcarbonylamino, dimethylaminosulphonyl,
   -T2-N(R611)R612, or —U-T3-N(R613)R614, in which
T2 is methylene, dimethylene or trimethylene,
R611 is methyl, cyclopropyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,
U is —O— (oxygen) or —C(O)NH—,
T3 is dimethylene or trimethylene,
R613 and R614 are methyl,
or R613 and R614 together and with inclusion of the nitrogen atom, to which they are bonded, form a heterocyclic ring Het2, in which Het2 is morpholino, piperidino, pyrrolidino or 4N-methyl-piperazino,

or
Q1 is substituted by R61 on the terminal ring, and is Hh1 or Ha1, in which
Hh1 is pyridinyl-thiophenyl,
Ha1 is 3-(pyridinyl)-phenyl or 4-(pyridinyl)-phenyl,
R61 is methoxy, or -T2-N(R611)R612, in which
T2 is a bond,
R611 and R612 are independently is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,

or
Q1 is 3-(1N-methyl-pyrazolyl)-phenyl, 4-(1N-methyl-pyrazolyl)-phenyl, 3-(1N-methyl-indolyl)-phenyl or 4-(1N-methyl-indolyl)-phenyl,
R7 is hydroxyl, or 2-aminophenyl,

and the salts of these compounds.

Yet compounds according to aspect B of the present invention to be emphasized are those compounds of formula I in which
R1, R2, R3, R4 and R5 are all hydrogen,
R6 is -T1-Q1, in which T1 is a bond,
Q is (1N-methyl-pyrazol-4-yl)-thiophenyl,
   3-(dimethyl-isoxazolyl)-phenyl or 4-(dimethyl-isoxazolyl)-phenyl,

and the salts of these compounds.

In another embodiment, still yet compounds according to aspect B of the present invention to be emphasized are those compounds of formula I in which
R1, R2, R3, R4 and R5 are all hydrogen,
R6 is -T1-Q1, in which T1 is a bond,
Q1 is 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is methoxy, hydroxyl, trifluoromethyl, hydroxymethyl, methylsulphonylamino, methylcarbonylamino, dimethylaminosulphonyl, -T2-N(R611)R612, or —U-T3-N(R613)R614, in which
T2 is a bond, methylene, dimethylene or trimethylene,
R611 is hydrogen, methyl, cyclopropyl, cyclopentyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,

either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, piperidino, pyrrolidino or 4N-methyl-piperazino,

or
U is —C(O)NH—,
T3 is dimethylene or trimethylene,
R613 and R614 are methyl,

or
Q1 is 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl, in which
R61 is methoxy, hydroxyl, trifluoromethyl, hydroxymethyl, methylsulphonylamino, methylcarbonylamino, dimethylaminosulphonyl, -T2-N(R611)R612, or —U-T3-N(R613)R614, in which T2 is a bond, methylene, dimethylene or trimethylene,

either
R611 is methyl, cyclopropyl, cyclopentyl or 2-methoxyethyl,
R612 is hydrogen,
or R611 and R612 are hydrogen,
or R611 and R612 are methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,

either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, piperidino, pyrrolidino or 4N-methyl-piperazino,

or
Q1 is 5-[2-(R61)-pyridin-4-yl]-thiophen-2-yl or 5-[6-(R61)-pyridin-3-yl]-thiophen-2-yl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino,

or
Q1 is 2-[2-(R61)-pyridin-4-yl]-pyridin-4-yl, 2-[6-(R61)-pyridin-3-yl]-pyridin-4-yl, 3-[2-(R61)-pyridin-4-yl]-pyridin-6-yl or 3-[6-(R61)-pyridin-3-yl]-pyridin-6-yl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino,

or
Q1 is 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino,

or
Q1 is 3-(1N-methyl-pyrazol-4-yl)-phenyl, 4-(1N-methyl-pyrazol-4-yl)-phenyl, 5-(1N-methyl-pyrazol-4-yl)-thiophen-2-yl, 6-(1N-methyl-pyrazol-4-yl)-pyridin-3-yl, 5-(2-methyl-thiazol-4-yl)-thiophen-2-yl, 3-(3,5-dimethyl-isoxazol-4-yl)-phenyl, 4-(3,5-dimethyl-isoxazol-4-yl)-phenyl, 3-(1N-methyl-indol-5-yl)-phenyl or 4-(1N-methyl-indol-5-yl)-phenyl,
R7 is hydroxyl, or 2-aminophenyl,

and the salts of these compounds.

Compounds according to aspect B of the present invention to be more emphasized are those compounds of formula I in which
R1, R2, R3, R4 and R5 are all hydrogen,
R6 is -T1-Q1, in which T1 is a bond,
Q1 is 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl,
   in which
R61 is hydroxymethyl, methylsulphonylamino, methylcarbonylamino, dimethylaminosulphonyl,
   -T2-N(R611)R612, or —U-T3-N(R613)R614, in which
T2 is methylene, dimethylene or trimethylene,
R611 is methyl, cyclopropyl or 2-methoxyethyl,
R612 is hydrogen or methyl,
or R611 and R612 together and with inclusion of the nitrogen atom, to which they are bonded, form a heterocyclic ring Het1, in which Het1 is morpholino, pyrrolidino or 4N-methyl-piperazino,

either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, pyrrolidino or 4N-methyl-piperazino,

or
U is —C(O)NH—,
T3 is dimethylene or trimethylene,
R613 and R614 are methyl,

or
Q1 is 5-[3-(R61)-phenyl]-thiophen-2-yl or 5-[4-(R61)-phenyl]-thiophen-2-yl, in which
R61 is -T2-N(R611)R612, or —U-T3-N(R613)R614, in which
T2 is methylene, dimethylene or trimethylene,
R611 and R612 are methyl,
or R611 and R612 together and with inclusion of the nitrogen atom, to which they are bonded, form a heterocyclic ring Het1, in which
Het1 is morpholino, pyrrolidino or 4N-methyl-piperazino,
U is —O— (oxygen),
T3 is dimethylene or trimethylene,
R613 and R614 together and with inclusion of the nitrogen atom, to which they are bonded, form a heterocyclic ring Het2, in which Het2 is morpholino, pyrrolidino or 4N-methyl-piperazino,

or
Q1 is 5-[2-(R61)-pyridin-4-yl]-thiophen-2-yl or 5-[6-(R61)-pyridin-3-yl]-thiophen-2-yl, in which R61 is amino, or -T2-N(R611)R612, in which T2 is a bond,
R611 and R612 together and with inclusion of the nitrogen atom, to which they are bonded, form a heterocyclic ring Het1, in which Het1 is morpholino, pyrrolidino or 4N-methyl-piperazino,
Q1 is 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is amino, methoxy, or -T2-N(R611)R612, in which T2 is a bond,
R611 and R612 together and with inclusion of the nitrogen atom, to which they are bonded, form a heterocyclic ring Het1, in which Het1 is morpholino, pyrrolidino or 4N-methyl-piperazino,

or
Q1 is 3-(1N-methyl-pyrazol-4-yl)-phenyl, 4-(1N-methyl-pyrazol-4-yl)-phenyl, 3-(1N-methyl-indol-5-yl)-phenyl or 4-(1N-methyl-indol-5-yl)-phenyl,
R7 is hydroxyl, or 2-aminophenyl,

and the salts of these compounds.

Yet compounds according to aspect B of the present invention to be more emphasized are those compounds of formula I in which
R1, R2, R3, R4 and R5 are all hydrogen,
R6 is -T1-Q1, in which T1 is a bond,
Q is 5-(1N-methyl-pyrazol-4-yl)-thiophen-2-yl, 3-(3,5-dimethyl-isoxazol-4-yl)-phenyl or 4-(3,5-dimethyl-isoxazol-4-yl)-phenyl,

and the salts of these compounds.

Compounds according to aspect B of the present invention to be in particular emphasized are those compounds of formula I in which
R1, R2, R3, R4 and R5 are all hydrogen,
R6 is -T1-Q1, in which T1 is a bond,
Q1 is any one selected from the group consisting of
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-ylmethyl)-biphenyl-3-yl, 4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 4′-dimethylsulphamoyl-biphenyl-4-yl, 3′-acetamido-biphenyl-4-yl, 4′-acetamido-biphenyl-4-yl, 4′-(2-methoxy-ethylamino)methyl-biphenyl-3-yl, 4′-cyclopropylaminomethyl-biphenyl-3-yl, 3′-hydroxymethyl-biphenyl-4-yl, 5-[2-(4-methyl-piperazin-1-yl)-pyridin-4-yl]-thiophen-2-yl, 5-(1N-methyl-pyrazol-4-yl)-thiophen-2-yl, 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 5-[4-(2-morpholin-4-yl-ethoxy)-phenyl]-thiophen-2-yl, 5-[4-(3-morpholin-4-yl-propoxy)-phenyl]-thiophen-2-yl, 5-{4-[2-(4-methyl-piperazin-1-yl)-ethoxy]-phenyl}-thiophen-2-yl, 5-(4-dimethylaminomethyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, 3-[6-amino-pyridin-3-yl]-phenyl, 4-[6-methoxy-pyridin-3-yl]-phenyl, 3-[6-methoxy-pyridin-3-yl]-phenyl, 3-(1N-methyl-pyrazol-4-yl)-phenyl, 4-(1N-methyl-pyrazol-4-yl)-phenyl, 4-(3,5-dimethyl-isoxazol-4-yl)-phenyl, and 4-(1N-methyl-indol-5-yl)-phenyl,
R7 is hydroxyl, or 2-aminophenyl,

and the salts of these compounds.

In one embodiment, compounds according to aspect B of the present invention to be in more particular emphasized are those compounds of formula I in which
R1, R2, R3, R4 and R5 are all hydrogen,
R6 is -T1-Q1, in which T1 is a bond,
Q1 is any one selected from the group consisting of
4′-(2-morpholin-4-yl-ethyl)-biphenyl-3-yl, 4′-(3-morpholin-4-yl-propoxy)-biphenyl-3-yl,
4′-[2-(4-methyl-piperazin-1-yl)-ethoxy]-biphenyl-3-yl, 4′-dimethylaminomethyl-biphenyl-4-yl,
5-[2-(4-methyl-piperazin-1-yl)-pyridin-4-yl]-thiophen-2-yl, 5-(4-dimethylaminomethyl-phenyl)-thiophen-2-yl,
4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, and 4-(1N-methyl-pyrazol-4-yl)-phenyl.

R7 is hydroxyl,

and the salts of these compounds.

In another embodiment, compounds according to aspect B of the present invention to be in more particular emphasized are those compounds of formula I in which
R1, R2, R3, R4 and R5 are all hydrogen,
R6 is -T1-Q1, in which T1 is a bond,
Q1 is any one selected from the group consisting of
4′-(2-morpholin-4-yl-ethyl)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-dimethylaminomethyl-biphenyl-4-yl, 5-[2-(4-methyl-piperazin-1-yl)-pyridin-4-yl]-thiophen-2-yl, 5-(4-dimethylaminomethyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, and 4-(1N-methyl-pyrazol-4-yl)-phenyl,
R7 is 2-aminophenyl,

and the salts of these compounds.

In a first embodiment of aspect C (embodiment C1) of the present invention, compounds according to aspect C of the present invention more worthy to be mentioned are those compounds of formula I in which
R1, R2, R3, R4, and R5 are independently hydrogen, or 1-4C-alkyl,
R6 is -T1-Q1, in which T1 is a bond,

either
Q1 is substituted by R61 and/or R62, and is Aa1, Hh1, Ha1, Ha2, Ha3 or Ah1,
or Q1 is unsubstituted, and is Ha2 or Ha3,

in which
R61 is 1-4C-alkyl, 1-4C-alkoxy, halogen, or -T2-N(R611)R612, in which
T2 is a bond or 1-4C-alkylene,
R611 and R612 are independently hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
R62 is 1-4C-alkyl,
Aa1 is biphenyl,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,

and the salts of these compounds.

In a second embodiment of aspect C (embodiment C2), compounds according to aspect C of the present invention more worthy to be mentioned are those compounds of formula I in which
R1, R2, R3, R4 and R5 are independently hydrogen, or 1-4C-alkyl,
R6 is -T1-Q1, in which T1 is a bond,

either
Q1 is substituted by R61, and is Aa1, Ha1, Ha2 or Ha3,
Or Q1 is unsubstituted, and is Ha2 or Ha3,

in which
R61 is 1-4C-alkyl, 1-4C-alkoxy, halogen, or -T2-N(R611)R612, in which
T2 is a bond or 1-4C-alkylene,
R611 and R612 are independently hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is biphenyl,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,

and the salts of these compounds.

Compounds according to embodiment C1 of aspect C of the present invention in particular worthy to be mentioned are those compounds of formula I in which
R1, R2, R3, R4 and R5 are hydrogen,
R6 is -T1-Q1, in which T1 is a bond,

either
Q1 is substituted by R61 on the terminal ring, and is Aa1, Hh1, Ha1 or Ah1,
or Q1 is [1N-(1-4C-alkyl)-indolyl]-phenyl, [1N-(1-4C-alkyl)-pyrazolyl]-phenyl, [1N-(1-4C-alkyl)-imidazolyl]-phenyl, [1N-(1-4C-alkyl)-triazolyl]-phenyl, [1N-(1-4C-alkyl)-tetrazolyl]-phenyl, [1N-(1-4C-alkyl)-benzimidazolyl]-phenyl, [1N-(1-4C-alkyl)-benztriazolyl]-phenyl, or [1N-(1-4C-alkyl)-indazol]-phenyl,
or Q1 is [1N-(1-4C-alkyl)-indolyl]-thiophenyl, [1N-(1-4C-alkyl)-pyrazolyl]-thiophenyl, [1N-(1-4C-alkyl)-imidazolyl]-thiophenyl, [1N-(1-4C-alkyl)-triazolyl]-thiophenyl, [1N-(1-4C-alkyl)-tetrazolyl]-thiophenyl, [1N-(1-4C-alkyl)-benzimidazolyl]-thiophenyl, [1N-(1-4C-alkyl)-benztriazolyl]-thiophenyl, or [1N-(1-4C-alkyl)-indazol]-thiophenyl,
or Q1 is [mono- or di-(1-4C-alkyl)-isoxazolyl]-phenyl, or [mono- or di-(1-4C-alkyl)-isoxazolyl]-thiophenyl,

in which
R61 is 1-4C-alkyl, 1-4C-alkoxy, halogen, or -T2-N(R611)R612, in which
T2 is a bond or 1-4C-alkylene,
R611 is hydrogen or 1-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is 1,1′-biphenyl-4-yl or 1,1′-biphenyl-3-yl,
Hh1 is pyridinyl-thiophenyl,
Ha1 is 3-(pyridinyl)-phenyl or 4-(pyridinyl)-phenyl,
Ah1 is phenyl-thiophenyl,
R7 is hydroxyl, or 2-aminophenyl,

and the salts of these compounds.

Compounds according to embodiment C2 of aspect C of the present invention in particular worthy to be mentioned are those compounds of formula I in which
R1, R2, R3, R4 and R5 are hydrogen,
R6 is -T1-Q1, in which T1 is a bond,

either
Q1 is substituted by R61 on the terminal ring, and is Aa1 or Ha1,
or Q1 is [1N-(1-4C-alkyl)-indolyl]-phenyl, [1N-(1-4C-alkyl)-pyrazolyl]-phenyl, [1N-(1-4C-alkyl)-imidazolyl]-phenyl, [1N-(1-4C-alkyl)-triazolyl]-phenyl, [1N-(1-4C-alkyl)-tetrazolyl]-phenyl, [1N-(1-4C-alkyl)-benzimidazolyl]-phenyl, [1N-(1-4C-alkyl)-benztriazolyl]-phenyl, or [1N-(1-4C-alkyl)-indazol]-phenyl,

in which
R61 is 1-4C-alkyl, 1-4C-alkoxy, halogen, or -T2-N(R611)R612, in which
T2 is a bond or 1-4C-alkylene,
R611 is hydrogen or 1-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is 1,1′-biphenyl-4-yl or 1,1′-biphenyl-3-yl,
Ha1 is 3-(pyridinyl)-phenyl or 4-(pyridinyl)-phenyl,
R7 is hydroxyl, or 2-aminophenyl,

and the salts of these compounds.

Compounds according to embodiment C1 of aspect C of the present invention in more particular worthy to be mentioned are those compounds of formula I in which
R1, R2, R3, R4 and R5 are hydrogen,
R6 is -T1-Q1, in which T1 is a bond,

either
Q1 is substituted by R61 on the pyridine ring, and is 3-(pyridinyl)-phenyl or 4-(pyridinyl)-phenyl,
or Q1 is 2′-(R61)-1,1′-biphenyl-4-yl, 3′-(R61)-1,1′-biphenyl-4-yl, 4′-(R61)-1,1′-biphenyl-4-yl, 2′-(R61)-1,1′-biphenyl-3-yl, 3′-(R61)-1,1′-biphenyl-3-yl or 4′-(R61)-1,1′-biphenyl-3-yl,
or Q1 is substituted by R61 on the pyridine ring, and is pyridinyl-thiophenyl,
or Q1 is substituted by R61 on the phenyl ring, and is phenyl-thiophenyl,
or Q1 is 3-[1N-methyl-indolyl]-phenyl, 4-[1N-methyl-indolyl]-phenyl, 3-[1N-methyl-pyrazolyl]-phenyl or 4-[1N-methyl-pyrazolyl]-phenyl,
or Q1 is [1N-methyl-pyrazolyl]-thiophenyl,
or Q1 is 3-[dimethyl-isoxazolyl]-phenyl or 4-[dimethyl-isoxazolyl]-phenyl,

in which
R61 is 1-2C-alkoxy, amino, or -T2-N(R611)R612, in which
T2 is a bond, methylene, dimethylene or trimethylene,
R611 is 1-2C-alkyl,
R612 is 1-2C-alkyl,
or R611 and R612 together and with inclusion of the nitrogen atom, to which they are bonded, form a heterocyclic ring Het1, in which
Het1 is morpholino, pyrrolidino or 4N-methyl-piperazino,
R7 is hydroxyl, or 2-aminophenyl,

and the salts of these compounds.

Compounds according to embodiment C2 of aspect C of the present invention in more particular worthy to be mentioned are those compounds of formula I in which
R1, R2, R3, R4 and R5 are hydrogen,
R6 is -T1-Q1, in which T1 is a bond,

either
Q1 is substituted by R61 on the pyridine ring, and is 3-(pyridinyl)-phenyl or 4-(pyridinyl)-phenyl,
or Q1 is 3′-(R61)-1,1′-biphenyl-4-yl or 4′-(R61)-1,1′-biphenyl-4-yl,
or Q1 is 3-[1N-methyl-indolyl]-phenyl, 4-[1N-methyl-indolyl]-phenyl, 3-[1N-methyl-pyrazolyl]-phenyl or 4-[1N-methyl-pyrazolyl]-phenyl,

in which
R61 is 1-2C-alkoxy, amino, or -T2-N(R611)R612, in which
T2 is a bond or 1-2C-alkylene,
R611 and R612 are 1-2C-alkyl,
or R611 and R612 together and with inclusion of the nitrogen atom, to which they are bonded, form a heterocyclic ring Het1, in which
Het1 is morpholino,
R7 is hydroxyl, or 2-aminophenyl,

and the salts of these compounds.

Compounds according to embodiment C1 of aspect C of the present invention to be emphasized are, in one embodiment, those compounds of formula in which
R1, R2, R3, R4 and R5 are hydrogen,
R6 is -T1-Q1, in which T1 is a bond,

either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3-[2-(4-methyl-piperazin-1-yl)-pyridin-4-yl)]-phenyl or 4-[2-(4-methyl-piperazin-1-yl)-pyridin-4-yl)]-phenyl,
or Q1 is 3′-(R61)-1,1′-biphenyl-4-yl or 4′-(R61)-1,1′-biphenyl-4-yl,
or Q1 is 5-[2-(4-methyl-piperazin-1-yl)-pyridin-4-yl)]-thiophen-2-yl,
or Q1 is 5-[4-(R61)-phenyl]-thiophen-2-yl or 5-[3-(R61)-phenyl]-thiophen-2-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,
or Q1 is 5-(1N-methyl-pyrazol-4-yl)-thiophen-2-yl,
or Q1 is 3-(3,5-dimethyl-isoxazol-4-yl)-phenyl or 4-(3,5-dimethyl-isoxazol-4-yl)-phenyl,

in which
R61 is -T2-N(R611)R612, in which
T2 is methylene, dimethylene or trimethylene,

either
R611 and R612 are both methyl,

or
R611 and R612 together and with inclusion of the nitrogen atom, to which they are bonded, form a heterocyclic ring Het1, in which
Het1 is morpholino or 4N-methyl-piperazino,
R7 is hydroxyl,

and the salts of these compounds.

Compounds according to embodiment C1 of aspect C of the present invention to be emphasized are, in another embodiment, those compounds of formula I in which
R1, R2, R3, R4 and R5 are hydrogen,
R6 is -T1-Q1, in which T1 is a bond,

either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3-[2-(4-methyl-piperazin-1-yl)-pyridin-4-yl)]-phenyl or 4-[2-(4-methyl-piperazin-1-yl)-pyridin-4-yl)]-phenyl,
or Q1 is 3′-(R61)-1,1′-biphenyl-4-yl or 4′-(R61)-1,1′-biphenyl-4-yl,
or Q1 is 5-[2-(4-methyl-piperazin-1-yl)-pyridin-4-yl)]-thiophen-2-yl,
or Q1 is 5-[4-(R61)-phenyl]-thiophen-2-yl or 5-[3-(R61)-phenyl]-thiophen-2-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,
or Q1 is 5-(1N-methyl-pyrazol-4-yl)-thiophen-2-yl,
or Q1 is 3-(3,5-dimethyl-isoxazol-4-yl)-phenyl or 4-(3,5-dimethyl-isoxazol-4-yl)-phenyl,

in which
R61 is -T2-N(R611)R612, in which T2 is methylene, dimethylene or trimethylene,

either
R611 and R612 are both methyl,

or
R611 and R612 together and with inclusion of the nitrogen atom, to which they are bonded, form a heterocyclic ring Het1, in which
Het1 is morpholino or 4N-methyl-piperazino,
R7 is 2-aminophenyl,

and the salts of these compounds.

Compounds according to embodiment C2 of aspect C of the present invention to be emphasized are those compounds of formula I in which
R1, R2, R3, R4 and R5 are hydrogen,
R6 is -T1-Q1, in which T1 is a bond,

either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3′-(R61)-1,1′-biphenyl-4-yl or 4′-(R61)-1,1′-biphenyl-4-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,

in which
R61 is -T2-N(R611)R612, in which T2 is 1-2C-alkylene,
R611 and R612 together and with inclusion of the nitrogen atom, to which they are bonded, form a heterocyclic ring Het1, in which Het1 is morpholino,
R7 is hydroxyl, or 2-aminophenyl,

and the salts of these compounds.

Compounds according to embodiment C1 of aspect C of the present invention to be more emphasized are, in one embodiment, those compounds of formula I in which
R1, R2, R3, R4 and R5 are hydrogen,
R6 is -T1-Q1, in which T1 is a bond,
Q1 is any one selected from 3-(6-amino-pyridin-3-yl)-phenyl, 4-(6-amino-pyridin-3-yl)-phenyl, 3-(6-methoxy-pyridin-3-yl)-phenyl, 4-(6-methoxy-pyridin-3-yl)-phenyl, 3-[2-(4-methyl-piperazin-1-yl)-pyridin-4-yl)]-phenyl, 4-[2-(4-methyl-piperazin-1-yl)-pyridin-4-yl)]-phenyl,
   3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-yl-methyl)-biphenyl-3-yl,
   2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 5-[2-(4-methyl-piperazin-1-yl)-pyridin-4-yl)]-thiophen-2-yl, 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 4-(1N-methyl-indol-5-yl)-phenyl, 3-(1N-methyl-pyrazol-4-yl)-phenyl, 4-(1N-methyl-pyrazol-4-yl)-phenyl, 5-(4-dimethylaminomethyl-phenyl)-thiophen-2-yl,
   5-(1N-methyl-pyrazol-4-yl)-thiophen-2-yl, and 4-(3,5-dimethyl-isoxazol-4-yl)-phenyl,
R7 is hydroxyl,

and the salts of these compounds.

Compounds according to embodiment C1 of aspect C of the present invention to be more emphasized are, in another embodiment, those compounds of formula I in which
R1, R2, R3, R4 and R5 are hydrogen,
R6 is -T1-Q1, in which T1 is a bond,
Q1 is any one selected from 3-(6-amino-pyridin-3-yl)-phenyl, 4-(6-amino-pyridin-3-yl)-phenyl, 3-(6-methoxy-pyridin-3-yl)-phenyl, 4-(6-methoxy-pyridin-3-yl)-phenyl, 3-[2-(4-methyl-piperazin-1-yl)-pyridin-4-yl)]-phenyl, 4-[2-(4-methyl-piperazin-1-yl)-pyridin-4-yl)]-phenyl, 3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-yl-methyl)-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 5-[2-(4-methyl-piperazin-1-yl)-pyridin-4-yl)]-thiophen-2-yl,
   5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 4-(1N-methyl-indol-5-yl)-phenyl, 3-(1N-methyl-pyrazol-4-yl)-phenyl, 4-(1N-methyl-pyrazol-4-yl)-phenyl,
   5-(4-dimethylaminomethyl-phenyl)-thiophen-2-yl, 5-(1N-methyl-pyrazol-4-yl)-thiophen-2-yl, and
   4-(3,5-dimethyl-isoxazol-4-yl)-phenyl,
R7 is 2-aminophenyl,

and the salts of these compounds.

Compounds according to embodiment C1 of aspect C of the present invention to be in particular emphasized are, in one embodiment, those compounds of formula I in which
R1, R2, R3, R4 and R5 are hydrogen,
R6 is -T1-Q1, in which T1 is a bond,
Q1 is any one selected from 4-(6-amino-pyridin-3-yl)-phenyl, 4-[2-(4-methyl-piperazin-1-yl)-pyridin-4-yl)]-phenyl, 3-[2-(4-methyl-piperazin-1-yl)-pyridin-4-yl)]-phenyl, 4′-(2-morpholin-4-yl-ethyl)-biphenyl-3-yl, 4′-dimethylaminomethyl-biphenyl-4-yl, 5-[2-(4-methyl-piperazin-1-yl)-pyridin-4-yl)]-thiophen-2-yl, 4-(1N-methyl-pyrazol-4-yl)-phenyl, and 5-(4-dimethylaminomethyl-phenyl)-thiophen-2-yl,
R7 is hydroxyl,

and the salts of these compounds.

Compounds according to embodiment C1 of aspect C of the present invention to be in particular emphasized are, in another embodiment, those compounds of formula I in which
R1, R2, R3, R4 and R5 are hydrogen,
R6 is -T1-Q1, in which T1 is a bond,
Q1 is any one selected from 4-(6-amino-pyridin-3-yl)-phenyl, 4-[2-(4-methyl-piperazin-1-yl)-pyridin-4-yl)]-phenyl, 3-[2-(4-methyl-piperazin-1-yl)-pyridin-4-yl)]-phenyl, 4′-(2-morpholin-4-yl-ethyl)-biphenyl-3-yl, 4′-dimethylaminomethyl-biphenyl-4-yl, 5-[2-(4-methyl-piperazin-1-yl)-pyridin-4-yl)]-thiophen-2-yl, 4-(1N-methyl-pyrazol-4-yl)-phenyl, and 5-(4-dimethylaminomethyl-phenyl)-thiophen-2-yl,
R7 is 2-aminophenyl,

and the salts of these compounds.

A special interest in the compounds according to the present invention refers to those compounds of this invention which are included—within the scope of this invention—by one or, when possible, a combination of more of the following embodiments: An embodiment of the compounds according to the present invention relates to those compounds of formula I, in which R1, R2, R3, R4 and R5 are all hydrogen.

A further embodiment of the compounds according to the present invention relates to those compounds of formula I, in which R7 is hydroxyl. A further embodiment of the compounds according to the present invention relates to those compounds of formula I, in which R7 is 2-aminophenyl. A further embodiment of the compounds according to the present invention relates to those compounds of formula I, in which R7 is aminopyridyl. A further embodiment of the compounds according to the present invention relates to those compounds of formula I, in which R7 is Cyc1, whereby in a subembodiment thereof Cyc1 is 2-phenyl.

A further embodiment of the compounds according to the present invention relates to those compounds of formula I, in which T1 is a bond.

A further embodiment of the compounds according to the present invention relates to those compounds of formula I, in which R6 is substituted by R61, and is Aa1, Ha1 or Ha2. A further embodiment of the compounds according to the present invention relates to those compounds of formula I, in which R6 is substituted by R61, and is Ah1 or Hh1. A further embodiment of the compounds according to the present invention relates to those compounds of formula I, in which R6 is substituted by R61, and is Ha3. A further embodiment of the compounds according to the present invention relates to those compounds of formula I, in which R6 is 3-(pyridinyl)-phenyl or 4-(pyridinyl)-phenyl, each of which is substituted by R61 on the pyridinyl moiety. A further embodiment of the compounds according to the present invention relates to those compounds of formula I, in which R6 is 3-(pyridin-3-yl)-phenyl, 3-(pyridin-4-yl)-phenyl, 4-(pyridin-3-yl)-phenyl, or 4-(pyridin-4-yl)-phenyl, each of which is substituted by R61 on the pyridinyl moiety. A further embodiment of the compounds according to the present invention relates to those compounds of formula I, in which R6 is 3-(pyridin-3-yl)-phenyl or 4-(pyridin-3-yl)-phenyl, each of which is substituted by R61 on the pyridinyl moiety. A further embodiment of the compounds according to the present invention relates to those compounds of formula I, in which R6 is 3-[6-(R61)-pyridin-3-yl]-phenyl or 4-[6-(R61)-pyridin-3-yl]-phenyl. A further embodiment of the compounds according to the present invention relates to those compounds of formula I, in which R6 is 3-(pyridin-4-yl)-phenyl or 4-(pyridin-4-yl)-phenyl, each of which is substituted by R61 on the pyridinyl moiety. A further embodiment of the compounds according to the present invention relates to those compounds of formula I, in which R6 is 3-[2-(R61)-pyridin-4-yl]-phenyl or 4-[2-(R61)-pyridin-4-yl]-phenyl. A further embodiment of the compounds according to the present invention relates to those compounds of formula I, in which R6 is 1,1′-biphenyl-4-yl or 1,1′-biphenyl-3-yl, each of which is substituted by R61 on the terminal phenyl moiety. A further embodiment of the compounds according to the present invention relates to those compounds of formula I, in which R6 is 3′-(R61)-1,1′-biphenyl-4-yl, 4′-(R61)-1,1′-biphenyl-4-yl, 3′-(R61)-1,1′-biphenyl-3-yl or 4′-(R61)-1,1′-biphenyl-3-yl. A further embodiment of the compounds according to the present invention relates to those compounds of formula I, in which R6 is 3′-(R61)-1,1′-biphenyl-4-yl or 4′-(R61)-1,1′-biphenyl-4-yl. A further embodiment of the compounds according to the present invention relates to those compounds of formula I, in which R6 is 3′-(R61)-1,1′-biphenyl-3-yl or 4′-(R61)-1,1′-biphenyl-3-yl.

A further embodiment of the compounds according to the present invention relates to those compounds of formula I, in which R6 is 4′-(R61)-1,1′-biphenyl-3-yl or 4′-(R61)-1,1′-biphenyl-4-yl.

A further embodiment of the compounds according to the present invention relates to those compounds of formula I, in which R6 is pyridinyl-thiophenyl, which is substituted by R61 on the pyridinyl moiety. A further embodiment of the compounds according to the present invention relates to those compounds of formula I, in which R6 is [2-(R61)-pyridin-4-yl]-thiophenyl, such as e.g. 5-[2-(R61)-pyridin-4-yl]-thiophen-2-yl. A further embodiment of the compounds according to the present invention relates to those compounds of formula I, in which R6 is [6-(R61)-pyridin-3-yl]-thiophenyl, such as e.g. 5-[6-(R61)-pyridin-3-yl]-thiophen-2-yl. A further embodiment of the compounds according to the present invention relates to those compounds of formula I, in which R6 is bipyridyl, which is substituted by R61 on the terminal pyridinyl moiety. A further embodiment of the compounds according to the present invention relates to those compounds of formula I, in which R6 is [2-(R61)-pyridin-4-yl]-pyridinyl, such as e.g. 2-[2-(R61)-pyridin-4-yl]-pyridin-4-yl or 6-[2-(R61)-pyridin-4-yl]-pyridin-3-yl. A further embodiment of the compounds according to the present invention relates to those compounds of formula I, in which R6 is [6-(R61)-pyridin-3-yl]-pyridinyl, such as e.g. 2-[6-(R61)-pyridin-3-yl]-pyridin-4-yl or 6-[6-(R61)-pyridin-3-yl]-pyridin-3-yl. A further embodiment of the compounds according to the present invention relates to those compounds of formula I, in which R6 is phenyl-thiophenyl, which is substituted by R61 on the phenyl moiety. A further embodiment of the compounds according to the present invention relates to those compounds of formula I, in which R6 is [3-(R61)-phenyl]-thiophenyl, such as e.g. 5-[3-(R61)-phenyl]-thiophen-2-yl. A further embodiment of the compounds according to the present invention relates to those compounds of formula I, in which R6 is [4-(R61)-phenyl]-thiophenyl, such as e.g. 5-[4-(R61)-phenyl]-thiophen-2-yl. A further embodiment of the compounds according to the present invention relates to those compounds of formula I, in which R6 is phenyl-pyridinyl, which is substituted by R61 on the phenyl moiety. A further embodiment of the compounds according to the present invention relates to those compounds of formula I, in which R6 is [3-(R61)-phenyl]-pyridinyl, such as e.g. 2-[3-(R61)-phenyl]-pyridin-4-yl or 6-[3-(R61)-phenyl]-pyridin-3-yl. A further embodiment of the compounds according to the present invention relates to those compounds of formula I, in which R6 is [4-(R61)-phenyl]-pyridinyl, such as e.g. 2-[4-(R61)-phenyl]-pyridin-4-yl or 6-[4-(R61)-phenyl]-pyridin-3-yl. A further embodiment of the compounds according to the present invention relates to those compounds of formula I, in which R6 is [1N-(1-4C-alkyl)-indolyl]-phenyl or [1N-(1-4C-alkyl)-pyrazolyl]-phenyl. A further embodiment of the compounds according to the present invention relates to those compounds of formula I, in which R6 is [1N-(1-2C-alkyl)-indol-5-yl]-phenyl or [1N-(1-2C-alkyl)-pyrazol-4-yl]-phenyl. A further embodiment of the compounds according to the present invention relates to those compounds of formula I, in which R6 is 3-(1N-methyl-pyrazol-4-yl)-phenyl or 4-(1N-methyl-pyrazol-4-yl)-phenyl. A further embodiment of the compounds according to the present invention relates to those compounds of formula I, in which R6 is [1N-(1-2C-alkyl)-pyrazol-4-yl]-pyridinyl, such as e.g. 2-(1N-methyl-pyrazol-4-yl)-pyridin-4-yl or 6-(1N-methyl-pyrazol-4-yl)-pyridin-3-yl. A further embodiment of the compounds according to the present invention relates to those compounds of formula I, in which R6 is triazolyl-phenyl, which is substituted by R61 on the triazolyl moiety. A further embodiment of the compounds according to the present invention relates to those compounds of formula I, in which R6 is {1N—(R61)-[1,2,3]triazol-4-yl}-phenyl, such as e.g. 3-{1N—(R61)-[1,2,3]triazol-4-yl}-phenyl or 4-{1N—(R61)-[1,2,3]triazol-4-yl}-phenyl. A further embodiment of the compounds according to the present invention relates to those compounds of formula I, in which R61 is -T2-N(R611)R612.

A further embodiment of the compounds according to the present invention relates to those compounds of formula I, in which T2 is a bond. A further embodiment of the compounds according to the present invention relates to those compounds of formula I, in which T2 is 1-4C-alkylene, such as e.g. 1-2C-alkylene. A further embodiment of the compounds according to the present invention relates to those compounds of formula I, in which T2 is methylene. A further embodiment of the compounds according to the present invention relates to those compounds of formula I, in which T2 is dimethylene.

A further embodiment of the compounds according to the present invention relates to those compounds of formula I, in which T2 is trimethylene. A further embodiment of the compounds according to the present invention relates to those compounds of formula I, in which R611 and R612 are both hydrogen.

A further embodiment of the compounds according to the present invention relates to those compounds of formula I, in which R611 and R612 are both methyl. A further embodiment of the compounds according to the present invention relates to those compounds of formula I, in which R611 and R612 together and with inclusion of the nitrogen atom, to which they are attached, form a morpholino ring. A further embodiment of the compounds according to the present invention relates to those compounds of formula I, in which R611 and R612 together and with inclusion of the nitrogen atom, to which they are attached, form a 4N-methyl-piperazino ring. A further embodiment of the compounds according to the present invention relates to those compounds of formula I, in which R611 and R612 together and with inclusion of the nitrogen atom, to which they are attached, form a pyrrolidino ring. A further embodiment of the compounds according to the present invention relates to those compounds of formula I, in which R611 and R612 together and with inclusion of the nitrogen atom, to which they are attached, form a piperidino ring. A further embodiment of the compounds according to the present invention relates to those compounds of formula I, in which R61 is —O-T3-N(R613)R614.

A further embodiment of the compounds according to the present invention relates to those compounds of formula I, in which T3 is dimethylene. A further embodiment of the compounds according to the present invention relates to those compounds of formula I, in which T3 is trimethylene.

A further embodiment of the compounds according to the present invention relates to those compounds of formula I, in which R613 and R614 are both methyl. A further embodiment of the compounds according to the present invention relates to those compounds of formula I, in which R613 and R614 together and with inclusion of the nitrogen atom, to which they are attached, form a morpholino ring. A further embodiment of the compounds according to the present invention relates to those compounds of formula I, in which R613 and R614 together and with inclusion of the nitrogen atom, to which they are attached, form a 4N-methyl-piperazino ring. A further embodiment of the compounds according to the present invention relates to those compounds of formula I, in which R613 and R614 together and with inclusion of the nitrogen atom, to which they are attached, form a pyrrolidino ring. A further embodiment of the compounds according to the present invention relates to those compounds of formula I, in which R613 and R614 together and with inclusion of the nitrogen atom, to which they are attached, form a piperidino ring.

A further embodiment of the compounds according to the present invention relates to those compounds of formula I, in which R61 is -T4-Het3, in which
T4 is a bond, methylene, dimethylene or trimethylene, and
Het3 is 1N-methyl-piperidin-4yl.

A further embodiment of the compounds according to the present invention relates to those compounds of formula I, in which R61 is —O-T5-Het4, in which
T5 is a bond, methylene, dimethylene or trimethylene, and
Het4 is 1N-methyl-piperidin-4yl.

A further embodiment of the compounds according to the present invention relates to those compounds of formula I, in which R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl; and R7 is hydroxyl.

A further embodiment of the compounds according to the present invention relates to those compounds of formula I, in which R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl; and R7 is 2-aminophenyl.

A further embodiment of the compounds according to the present invention relates to those compounds of formula I, in which R6 is 4-(6-amino-pyridin-3-yl)-phenyl. A further embodiment of the compounds according to the present invention relates to those compounds of formula I, in which R6 is 4-[2-(4-methyl-piperazin-1-yl)-pyridin-4-yl)]-phenyl. A further embodiment of the compounds according to the present invention relates to those compounds of formula I, in which R6 is 3-[2-(4-methyl-piperazin-1-yl)-pyridin-4-yl)]-phenyl. A further embodiment of the compounds according to the present invention relates to those compounds of formula I, in which R6 is 4′-(2-morpholin-4-yl-ethyl)-biphenyl-3-yl. A further embodiment of the compounds according to the present invention relates to those compounds of formula I, in which R6 is 4′-dimethylaminomethyl-biphenyl-4-yl. A further embodiment of the compounds according to the present invention relates to those compounds of formula I, in which R6 is 5-[2-(4-methyl-piperazin-1-yl)-pyridin-4-yl)]-thiophen-2-yl. A further embodiment of the compounds according to the present invention relates to those compounds of formula I, in which R6 is 5-(4-dimethylaminomethyl-phenyl)-thiophen-2-yl.

A special embodiment of the compounds according to the present invention relates to those compounds of formula I, in which R1, R2, R3, R4 and R5 are all hydrogen, and R7 is hydroxyl. Another special embodiment of the compounds according to the present invention relates to those compounds of formula I, in which R1, R2, R3, R4 and R5 are all hydrogen, and R7 is 2-aminophenyl.

It is to be understood, that the present invention also includes any or all possible combinations and subsets of the embodiments defined herein afore.

Exemplary compounds according to this invention may include any one selected from
1. (E)-N-Hydroxy-3-{1-[4-(1-methyl-1H-indol-5-yl)-benzenesulfonyl]-1H-pyrrol-3-yl}-acrylamide,
2. (E)-N-Hydroxy-3-{1-[4-(1-methyl-1H-pyrazol-4-yl)-benzenesulfonyl]-1H-pyrrol-3-yl}-acrylamide,
3. (E)-N-Hydroxy-3-{1-[4-(6-methoxy-pyridin-3-yl)-benzenesulfonyl]-1H-pyrrol-3-yl}-acrylamide,
4. (E)-3-{1-[4-(6-Amino-pyridin-3-yl)-benzenesulfonyl]-1H-pyrrol-3-yl}-N-hydroxy-acrylamide,
5. (E)-N-(2-Amino-phenyl)-3-{1-[4-(6-methoxy-pyridin-3-yl)-benzenesulfonyl]-1H-pyrrol-3-yl}-acrylamide,
6. (E)-N-(2-Amino-phenyl)-3-{1-[4-(6-amino-pyridin-3-yl)-benzenesulfonyl]-1H-pyrrol-3-yl}-acrylamide,
7. (E)-N-(2-Amino-phenyl)-3-{1-[4-(1-methyl-1H-pyrazol-4-yl)-benzenesulfonyl]-1H-pyrrol-3-yl}-acrylamide,
8. (E)-N-Hydroxy-3-{1-[4′-(2-morpholin-4-yl-ethyl)-biphenyl-4-sulfonyl]-1H-pyrrol-3-yl}-acrylamide,
9. (E)-N-Hydroxy-3-{1-[3′-(2-morpholin-4-yl-ethyl)-biphenyl-4-sulfonyl]-1H-pyrrol-3-yl}-acrylamide,
10. (E)-3-{1-[3-(6-Amino-pyridin-3-yl)-benzenesulfonyl]-1H-pyrrol-3-yl}-N-hydroxy-acrylamide,
11. (E)-N-Hydroxy-3-{1-[3-(6-methoxy-pyridin-3-yl)-benzenesulfonyl]-1H-pyrrol-3-yl}-acrylamide,
12. (E)-N-Hydroxy-3-{1-[3-(1-methyl-1H-pyrazol-4-yl)-benzenesulfonyl]-1H-pyrrol-3-yl}-acrylamide,
13. (E)-N-Hydroxy-3-{1-[3-(1-methyl-1H-indol-5-yl)-benzenesulfonyl]-1H-pyrrol-3-yl}-acrylamide,
14. (E)-N-(2-Amino-phenyl)-3-{1-[3-(6-methoxy-pyridin-3-yl)-benzenesulfonyl]-1H-pyrrol-3-yl}-acrylamide,
15. (E)-N-(2-Amino-phenyl)-3-{1-[3-(1-methyl-1H-pyrazol-4-yl)-benzenesulfonyl]-1H-pyrrol-3-yl}-acrylamide,
16. (E)-N-Hydroxy-3-{1-[4′-(2-morpholin-4-yl-ethyl)-biphenyl-3-sulfonyl]-1H-pyrrol-3-yl}-acrylamide,
17. (E)-N-(2-Amino-phenyl)-3-{1-[3-(6-amino-pyridin-3-yl)-benzenesulfonyl]-1H-pyrrol-3-yl}-acrylamide,
18. (E)-N-(2-Amino-phenyl)-3-{1-[3′-(2-morpholin-4-yl-ethyl)-biphenyl-3-sulfonyl]-1H-pyrrol-3-yl}-acrylamide,
19. (E)-N-(2-Amino-phenyl)-3-{1-[4′-(2-morpholin-4-yl-ethyl)-biphenyl-3-sulfonyl]-1H-pyrrol-3-yl}-acrylamide,
20. (E)-N-Hydroxy-3-{1-[3′-(2-morpholin-4-yl-ethyl)-biphenyl-3-sulfonyl]-1H-pyrrol-3-yl}-acrylamide,
21. (E)-N-Hydroxy-3-[1-(2′-methanesulfonylamino-biphenyl-4-sulfonyl)-1H-pyrrol-3-yl]-acrylamide
22. (E)-N-hydroxy-3-[1-(3′-methanesulfonylamino-biphenyl-4-sulfonyl)-1H-pyrrol-3-yl]-acrylamide
23. (E)-N-Hydroxy-3-[1-(4′-methanesulfonylamino-biphenyl-4-sulfonyl)-1H-pyrrol-3-yl]-acrylamide
24. 4′-[3-((E)-2-Hydroxycarbamoyl-vinyl)-pyrrole-1-sulfonyl]-biphenyl-4-carboxylic acid (2-dimethylamino-ethyl)-amide,
25. 4′-[3-((E)-2-Hydroxycarbamoyl-vinyl)-pyrrole-1-sulfonyl]-biphenyl-3-carboxylic acid (2-dimethylamino-ethyl)-amide,
26. (E)-3-[1-(4′-Dimethylaminomethyl-biphenyl-4-sulfonyl)-1H-pyrrol-3-yl]-N-hydroxy-acrylamide,
27. (E)-3-[1-(2′-Dimethylaminomethyl-biphenyl-4-sulfonyl)-1H-pyrrol-3-yl]-N-hydroxy-acrylamide,
28. (E)-N-Hydroxy-3-(1-{4-[2-(4-methyl-piperazin-1-yl)-pyridin-4-yl]-benzenesulfonyl}-1H-pyrrol-3-yl)-acrylamide,
29. (E)-N-Hydroxy-3-{1-[4′-(toluene-4-sulfonylamino)-biphenyl-4-sulfonyl]-1H-pyrrol-3-yl}-acrylamide,
30. 3′-[3-((E)-2-Hydroxycarbamoyl-vinyl)-pyrrole-1-sulfonyl]-biphenyl-4-carboxylic acid (2-dimethylamino-ethyl)-amide,
31. (E)-N-Hydroxy-3-[1-(3′-morpholin-4-ylmethyl-biphenyl-3-sulfonyl)-1H-pyrrol-3-yl]-acrylamide,
32. (E)-N-Hydroxy-3-(1-{4′-[2-(4-methyl-piperazin-1-yl)-ethoxy]-biphenyl-3-sulfonyl}-1H-pyrrol-3-yl)-acrylamide,
33. (E)-N-Hydroxy-3-(1-{3-[2-(4-methyl-piperazin-1-yl)-pyridin-4-yl]-benzenesulfonyl}-1H-pyrrol-3-yl)-acrylamide,
34. (E)-N-Hydroxy-3-{1-[4′-(2-morpholin-4-yl-ethoxy)-biphenyl-3-sulfonyl]-1H-pyrrol-3-yl}-acrylamide,
35. (E)-N-(2-Amino-phenyl)-3-{1-[4-(1-benzyl-1H-pyrazol-4-yl)-benzenesulfonyl]-1H-pyrrol-3-yl}-acrylamide,
36. (E)-N-Hydroxy-3-[1-(4′-morpholin-4-ylmethyl-biphenyl-3-sulfonyl)-1H-pyrrol-3-yl]-acrylamide,
37. (E)-3-[1-(4′-Dimethylaminomethyl-biphenyl-3-sulfonyl)-1H-pyrrol-3-yl]-N-hydroxy-acrylamide,
38. (E)-N-Hydroxy-3-{1-[4′-(3-morpholin-4-yl-propoxy)-biphenyl-3-sulfonyl]-1H-pyrrol-3-yl}-acrylamide,
39. (E)-N-(2-Amino-phenyl)-3-[1-(4′-dimethylsulfamoyl-biphenyl-4-sulfonyl)-1H-pyrrol-3-yl]-acrylamide,
40. (E)-3-[1-(3′-Acetylamino-biphenyl-4-sulfonyl)-1H-pyrrol-3-yl]-N-(2-amino-phenyl)-acrylamide,
41. (E)-3-[1-(2′-Dimethylaminomethyl-biphenyl-3-sulfonyl)-1H-pyrrol-3-yl]-N-hydroxy-acrylamide,
42. (E)-N-Hydroxy-3-(1-{5-[2-(4-methyl-piperazin-1-yl)-pyridin-4-yl]-thiophene-2-sulfonyl}-1H-pyrrol-3-yl)-acrylamide,
43. (E)-N-Hydroxy-3-{1-[4′-(2-pyrrolidin-1-yl-ethoxy)-biphenyl-3-sulfonyl]-1H-pyrrol-3-yl}-acrylamide,
44. 4′-{3-[(E)-2-(2-Amino-phenylcarbamoyl)-vinyl]-pyrrole-1-sulfonyl}-biphenyl-3-carboxylic acid (2-dimethylamino-ethyl)-amide,
45. (E)-N-Hydroxy-3-{1-[4′-(3-morpholin-4-yl-propyl)-biphenyl-3-sulfonyl]-1H-pyrrol-3-yl}-acrylamide,
46. (E)-3-{1-[5-(4-Dimethylaminomethyl-phenyl)-thiophene-2-sulfonyl]-1H-pyrrol-3-yl}-N-hydroxy-acrylamide,
47. (E)-N-(2-Amino-phenyl)-3-[1-(4′-dimethylaminomethyl-biphenyl-4-sulfonyl)-1H-pyrrol-3-yl]-acrylamide,
48. (E)-N-(2-Amino-phenyl)-3-(1-{4-[2-(4-methyl-piperazin-1-yl)-pyridin-4-yl]-benzenesulfonyl}-1H-pyrrol-3-yl)-acrylamide,
49. (E)-3-[1-(4′-Acetylamino-biphenyl-4-sulfonyl)-1H-pyrrol-3-yl]-N-(2-amino-phenyl)-acrylamide,
50. (E)-N-Hydroxy-3-{1-[5-(3-morpholin-4-ylmethyl-phenyl)-thiophene-2-sulfonyl]-1H-pyrrol-3-yl}-acrylamide,
51. (E)-N-(2-Amino-phenyl)-3-[1-(3′-hydroxymethyl-biphenyl-4-sulfonyl)-1H-pyrrol-3-yl]-acrylamide,
52. (E)-N-(2-Amino-phenyl)-3-{1-[4-(3,5-dimethyl-isoxazol-4-yl)-benzenesulfonyl]-1H-pyrrol-3-yl}-acrylamide,
53. (E)-N-(2-Amino-phenyl)-3-[1-(4′-methanesulfonylamino-biphenyl-4-sulfonyl)-1H-pyrrol-3-yl]-acrylamide,
54. (E)-N-Hydroxy-3-{1-[5-(4-morpholin-4-ylmethyl-phenyl)-thiophene-2-sulfonyl]-1H-pyrrol-3-yl}-acrylamide,
55. (E)-N-Hydroxy-3-[1-(5-{4-[2-(4-methyl-piperazin-1-yl)-ethoxy]-phenyl}-thiophene-2-sulfonyl)-1H-pyrrol-3-yl]-acrylamide,
56. (E)-N-Hydroxy-3-(1-{5-[4-(2-morpholin-4-yl-ethoxy)-phenyl]-thiophene-2-sulfonyl}-1H-pyrrol-3-yl)-acrylamide,
57. (E)-N-Hydroxy-3-(1-{5-[4-(3-morpholin-4-yl-propoxy)-phenyl]-thiophene-2-sulfonyl}-1H-pyrrol-3-yl)-acrylamide,
58. (E)-N-Hydroxy-3-(1-{4′-[(2-methoxy-ethylamino)-methyl]-biphenyl-3-sulfonyl}-1H-pyrrol-3-yl)-acrylamide,
59. (E)-N-(2-Amino-phenyl)-3-[1-(3′-methanesulfonylamino-biphenyl-4-sulfonyl)-1H-pyrrol-3-yl]-acrylamide,
60. (E)-Hydroxy-3-{1-[5-(1-methyl-1H-pyrazol-4-yl)-thiophene-2-sulfonyl]-1H-pyrrol-3-yl}-acrylamide,
61. (E)-N-Hydroxy-3-(1-{5-[4-(2-morpholin-4-yl-ethyl)-phenyl]-thiophene-2-sulfonyl}-1H-pyrrol-3-yl)-acrylamide,
62. (E)-N-Hydroxy-3-{1-[4′-(4-methyl-piperazin-1-ylmethyl)-biphenyl-3-sulfonyl]-1H-pyrrol-3-yl}-acrylamide, and
63. (E)-3-[1-(4′-Cyclopropylaminomethyl-biphenyl-3-sulfonyl)-1H-pyrrol-3-yl]-N-hydroxy-acrylamide,

and the salts thereof.

Further on, exemplary compounds according to this invention may also include any one selected from
64. (E)-N-Hydroxy-3-[1-(3′-morpholin-4-ylmethyl-biphenyl-4-sulfonyl)-1H-pyrrol-3-yl]-acrylamide,
65. (E)-3-[1-(4-Benzo[1,3]dioxol-5-yl-benzenesulfonyl)-1H-pyrrol-3-yl]-N-hydroxy-acrylamide,
66. (E)-3-[1-(3′-Amino-biphenyl-4-sulfonyl)-1H-pyrrol-3-yl]-N-hydroxy-acrylamide,
67. (E)-N-Hydroxy-3-[1-(4′-hydroxy-biphenyl-4-sulfonyl)-1H-pyrrol-3-yl]-acrylamide,
68. (E)-N-Hydroxy-3-(1-{4′-[2-(1-methyl-piperidin-4-yl)-ethoxy]-biphenyl-4-sulfonyl}-1H-pyrrol-3-yl)-acrylamide,
69. (E)-3-[1-(3′-Dimethylamino-biphenyl-4-sulfonyl)-1H-pyrrol-3-yl]-N-hydroxy-acrylamide,
70. (E)-3-{1-[4-(2,3-Dihydro-benzofuran-5-yl)-benzenesulfonyl]-1H-pyrrol-3-yl}-N-hydroxy-acrylamide,
71. (E)-N-Hydroxy-3-[1-(4′-morpholin-4-yl-biphenyl-4-sulfonyl)-1H-pyrrol-3-yl]-acrylamide,
72. (E)-N-Hydroxy-3-{1-[3′-(3-pyrrolidin-1-yl-propoxy)-biphenyl-4-sulfonyl]-1H-pyrrol-3-yl}-acrylamide,
73. (E)-N-Hydroxy-3-(1-{3′-[3-(4-methyl-piperazin-1-yl)-propoxy]-biphenyl-4-sulfonyl}-1H-pyrrol-3-yl)-acrylamide,
74. (E)-N-Hydroxy-3-{1-[3′-(3-morpholin-4-yl-propoxy)-biphenyl-4-sulfonyl]-1H-pyrrol-3-yl}-acrylamide,
75. (E)-N-Hydroxy-3-[1-(3′-morpholin-4-ylmethyl-biphenyl-4-sulfonyl)-1H-pyrrol-3-yl]-acrylamide,
76. (E)-N-Hydroxy-3-(1-{4′-[2-(4-methyl-piperazin-1-yl)-ethoxy]-biphenyl-4-sulfonyl}-1H-pyrrol-3-yl)-acrylamide,
77. (E)-N-Hydroxy-3-{1-[4′-(2-morpholin-4-yl-ethoxy)-biphenyl-4-sulfonyl]-1H-pyrrol-3-yl}-acrylamide,
78. (E)-N-Hydroxy-3-{1-[4′-(3-morpholin-4-yl-propoxy)-biphenyl-4-sulfonyl]-1H-pyrrol-3-yl}-acrylamide,
79. (E)-N-Hydroxy-3-(1-{4′-[3-(4-methyl-piperazin-1-yl)-propoxy]-biphenyl-4-sulfonyl}-1H-pyrrol-3-yl)-acrylamide,
80. (E)-N-Hydroxy-3-{1-[3′-(2-pyrrolidin-1-yl-ethoxy)-biphenyl-4-sulfonyl]-1H-pyrrol-3-yl}-acrylamide,
81. (E)-N-Hydroxy-3-{1-[4′-(3-pyrrolidin-1-yl-propoxy)-biphenyl-4-sulfonyl]-1H-pyrrol-3-yl}-acrylamide,
82. (E)-N-Hydroxy-3-[1-(4′-methoxy-biphenyl-4-sulfonyl)-1H-pyrrol-3-yl]-acrylamide,
83. (E)-N-Hydroxy-3-(1-{4-[1-(2-morpholin-4-yl-ethyl)-1H-[1,2,3]triazol-4-yl]-benzenesulfonyl}-1H-pyrrol-3-yl)-acrylamide,
84. (E)-3-[1-(4′-Cyclopentylaminomethyl-biphenyl-4-sulfonyl)-1H-pyrrol-3-yl]-N-hydroxy-acrylamide,
85. (E)-N-Hydroxy-3-[1-(3′-trifluoromethyl-biphenyl-4-sulfonyl)-1H-pyrrol-3-yl]-acrylamide,
86. (E-3-{1-[5-(3-Dimethylaminomethyl-phenyl)-thiophene-2-sulfonyl]-1H-pyrrol-3-yl}-N-hydroxy-acrylamide,
87. (E)-3-[1-(3′-Dimethylaminomethyl-biphenyl-3-sulfonyl)-1H-pyrrol-3-yl]-N-hydroxy-acrylamide,
88. (E)-N-Hydroxy-3-{1-[4′-(2-morpholin-4-yl-ethyl)-biphenyl-3-sulfonyl]-1H-pyrrol-3-yl}-acrylamide,
89. (E)-N-(2-Amino-phenyl)-3-{1-[6-(4-dimethylaminomethyl-phenyl)-pyridine-3-sulfonyl]-1H-pyrrol-3-yl}-acrylamide,
90. (E)-N-Hydroxy-3-{1-[5-(2-methyl-thiazol-4-yl)-thiophene-2-sulfonyl]-1H-pyrrol-3-yl}-acrylamide,
91. (E)-3-[1-(4′-Aminomethyl-biphenyl-3-sulfonyl)-1H-pyrrol-3-yl]-N-hydroxy-acrylamide,
92. (E)-N-Hydroxy-3-(1-{6-[4-(2-pyrrolidin-1-yl-ethoxy)-phenyl]-pyridine-3-sulfonyl}-1H-pyrrol-3-yl)-acrylamide,
93. (E)-3-[1-(4′-Aminomethyl-biphenyl-4-sulfonyl)-1H-pyrrol-3-yl]-N-(2-amino-phenyl)-acrylamide,
94. (E)-3-{1-[5-(3-Aminomethyl-phenyl)-thiophene-2-sulfonyl]-1H-pyrrol-3-yl}-N-hydroxy-acrylamide,
95. (E)-N-(2-Amino-phenyl)-3-{1-[5-(4-dimethylaminomethyl-phenyl)-thiophene-2-sulfonyl]-1H-pyrrol-3-yl}-acrylamide,
96. (E)-N-(2-Amino-phenyl)-3-[1-(3′-dimethylaminomethyl-biphenyl-4-sulfonyl)-1H-pyrrol-3-yl]-acrylamide,
97. (E)-3-{1-[4′-(Acetylamino-methyl)-biphenyl-4-sulfonyl]-1H-pyrrol-3-yl}-N-(2-amino-phenyl)-acrylamide,
98. (E)-N-(2-Amino-phenyl)-3-{1-[4′-(methanesulfonylamino-methyl)-biphenyl-4-sulfonyl]-1H-pyrrol-3-yl}-acrylamide,
99. (E)-N-Hydroxy-3-(1-{5-[4-(2-pyrrolidin-1-yl-ethoxy)-phenyl]-thiophene-2-sulfonyl}-1H-pyrrol-3-yl)-acrylamide,
100. (E)-3-{1-[5-(4-Dimethylsulfamoyl-phenyl)-thiophene-2-sulfonyl]-1H-pyrrol-3-yl}-N-hydroxy-acrylamide,
101. (E)-N-(2-Amino-phenyl)-3-[1-(4′-methanesulfonylamino-biphenyl-3-sulfonyl)-1H-pyrrol-3-yl]-acrylamide,
102. (E)-N-(2-Amino-phenyl)-3-[1-(4′-dimethylaminomethyl-biphenyl-3-sulfonyl)-1H-pyrrol-3-yl]-acrylamide,
103. (E)-N-Hydroxy-3-{1-[2′-(4-methyl-piperazin-1-yl)-[2,4′]bipyridinyl-5-sulfonyl]-1H-pyrrol-3-yl}-acrylamide,
104. (E)-N-(2-Amino-phenyl)-3-{1-[5-(1-methyl-1H-pyrazol-4-yl)-thiophene-2-sulfonyl]-1H-pyrrol-3-yl}-acrylamide,
105. (E)-3-{1-[6-(4-Dimethylaminomethyl-phenyl)-pyridine-3-sulfonyl]-1H-pyrrol-3-yl}-N-hydroxy-acrylamide,
106. (E)-N-(2-Amino-phenyl)-3-(1-{5-[2-(4-methyl-piperazin-1-yl)-pyridin-4-yl]-thiophene-2-sulfonyl}-1H-pyrrol-3-yl)-acrylamide,
107. (E)-N-(2-Amino-phenyl)-3-[1-(4′-morpholin-4-ylmethyl-biphenyl-4-sulfonyl)-1H-pyrrol-3-yl]-acrylamide,
108. (E)-N-(2-Amino-phenyl)-3-{1-[4′-(2-pyrrolidin-1-yl-ethoxy)-biphenyl-4-sulfonyl]-1H-pyrrol-3-yl}-acrylamide,
109. (E)-N-Hydroxy-3-(1-{4-[1-(2-piperidin-1-yl-ethyl)-1H-[1,2,3]triazol-4-yl]-benzenesulfonyl}-1H-pyrrol-3-yl)-acrylamide,
110. (E)-3-[1-(3′-Dimethylaminomethyl-biphenyl-3-sulfonyl)-1H-pyrrol-3-yl]-N-hydroxy-acrylamide,
111. (E)-N-(2-Amino-phenyl)-3-(1-{5-[4-(methynesulfonylamino-methyl)-phenyl]-thiophene-2-sulfonyl}-1H-pyrrol-3-yl)-acrylamide,
112. (E)-N-(2-Amino-phenyl)-3-{1-[3′-(methanesulfonylamino-methyl)-biphenyl-3-sulfonyl]-1H-pyrrol-3-yl}-acrylamide,
113. (E)-3-(1-{5-[4-(Acetylamino-methyl)-phenyl]-thiophene-2-sulfonyl}-1H-pyrrol-3-yl)-N-(2-amino-phenyl)-acrylamide,
114. (E)-N-(2-Amino-phenyl)-3-{1-[5-(3-dimethylaminomethyl-phenyl)-thiophene-2-sulfonyl]-1H-pyrrol-3-yl}-acrylamide,
115. (E)-N-(2-Amino-phenyl)-3-[1-(3′-dimethylaminomethyl-biphenyl-3-sulfonyl)-1H-pyrrol-3-yl]-acrylamide,
116. (E)-3-[1-(3′-Dimethylaminomethyl-biphenyl-4-sulfonyl)-1H-pyrrol-3-yl]-N-hydroxy-acrylamide,
117. (E)-3-{1-[5-(3-Dimethylaminomethyl-phenyl)-thiophene-2-sulfonyl]-1H-pyrrol-3-yl}-N-hydroxy-acrylamide,
118. (E)-3-{1-[3′-(Acetylamino-methyl)-biphenyl-3-sulfonyl]-1H-pyrrol-3-yl}-N-(2-amino-phenyl)-acrylamide,
119. (E)-N-(2-Amino-phenyl)-3-{1-[6-(1-methyl-1H-pyrazol-4-yl)-pyridine-3-sulfonyl]-1H-pyrrol-3-yl}-acrylamide,
120. (E)-N-Hydroxy-3-{1-[6-(1-methyl-1H-pyrazol-4-yl)-pyridine-3-sulfonyl]-1H-pyrrol-3-yl}-acrylamide, and
121. (E)-3-{1-[6-(3-Dimethylaminomethyl-phenyl)-pyridine-3-sulfonyl]-1H-pyrrol-3-yl}-N-hydroxy-acrylamide,

and the salts thereof.

In an embodiment of the foregoing, exemplary compounds according to this invention may especially include any one selected from the group consisting of the compounds 2, 4, 7, 16, 26, 28, 32, 33, 38, 42 and 46 as mentioned afore, and the salts thereof.

As used herein, 4SC-202 and (E)-N-(2-aminophenyl)-3-(1-((4-(1-methyl-1H-pyrazol-4-yl)phenyl)sulfonyl)-1H-pyrrol-3-yl)acrylamide (its chemical name) are used interchangeably and both refer to a compound of the following formula:

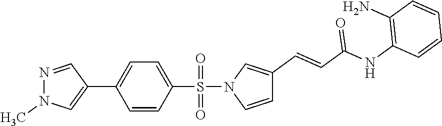 [SMILES: Cn1cc(-c2ccc(S(=O)(=O)n3ccc(/C=C/C(=O)Nc4ccccc4N)c3)cc2)cn1]

Suitable salts for the HDAC inhibitor are acid addition salts or salts with bases. Particular mention may be made of the pharmacologically tolerable inorganic and organic acids and bases customarily used in pharmacy. Those suitable are, on the one hand, water-insoluble and, particularly, water-soluble acid addition salts, the acids being employed in salt preparation in an equimolar quantitative ratio or one differing therefrom, particularly in an equimolar quantitative ratio. On the other hand, salts with bases are—depending on substitution—also suitable, the bases being employed in salt preparation in an equimolar quantitative ratio or one differing therefrom. Pharmacologically intolerable salts, which can be obtained, for example, as process products during the preparation of the HDAC inhibitor on an industrial scale, are converted into pharmacologically tolerable salts by processes known to the person skilled in the art. According to the invention, the HDAC inhibitor as well as its salts may contain, e.g. when isolated in crystalline form, varying amounts of solvents. Included within the scope of the present invention are therefore all solvates and in particular all hydrates of the HDAC inhibitor as well as all solvates and in particular all hydrates of the HDAC inhibitor, in particular such solvates or hydrates comprising about 0.5, 1 or 2 solvate or water molecules per molecule of the HDAC inhibitor or salts thereof.

Particular salts in the context of the present invention are the salts of 4SC-202 with, HBr, methansulfonic acid, hemiethane-1,2-disulfonic acid, benzenesulfonic acid, toluenesulfonic acid and 2-naphthalenesulfonic acid, more particularly toluenesulfonic acid, in particular in a molar ratio of about 1:1.

The HDAC inhibitor, in particular 4SC-202 and salts thereof can be prepared, for example, as described in detail in WO 2006/097474 A1 and WO 2009/112522 A1, respectively.

Particular PD-1 inhibitors in the context of the present invention are Pembrolizumab (Keytruda) and Nivolumab (Opdivo); Particular PD-L1 inhibitors in the context of the present invention are Atezolizumab (Tecentriq), Avelumab (Bavencio) and Durvalumab (Imfinzi); a particular CTLA-4 inhibitor in the context of the present invention is Ipilimumab (Yervoy).

The ability of immune checkpoint modulators which are biologicals to bind their respective targets (e.g. PD-1, PD-L1, PD-L2 or CTLA-4) can be assessed by in vitro/in vivo and/or cell-based assays either using purified domains of the target proteins or cells using ELISA or flow cytometry methods with a wide array of assays, e.g. the ELISA assay as described herein. Similarly, the ability of the antibodies to block the interaction with their respective ligands or in general can generate a biological response can be evaluated in a similar way (in vitro and/or cell-based) using designated ligand/receptor binding systems or biological assays. Exemplary methods for in vitro characterization of immune checkpoint modulators are described in: Cancer Immunol Res. 2014 September; 2(9): 846-56 and Cancer Immunol Res. 2015 September; 3(9): 1052-62.

Several of the specific immune checkpoint modulators described herein are commercially available and methods of their preparation are available from the related literature.

Pembrolizumab is a humanized monoclonal PD-1 antibody (IgG4 kappa isotype with class-II mechanism of action)) produced in Chinese hamster ovary cells by recombinant DNA technology. The typical preparation provided is a vial of powder containing 50 mg of Pembrolizumab, to be reconstituted (typically with sterile deionized water (e.g. milliQ grade) or aqueous NaCl 0.9, or glucose 5% solution) to a concentrate containing 25 mg/ml Pembrolizumab. Pembrolizumab is typically to be administered intravenously over 30 minutes in an infusion volume of typically 100-200 ml (typically sterile aqueous NaCl 0.9, or glucose 5% solution) (or typically to a concentration of 1 to 10 mg/ml).

The biological and medicinal properties of the HDAC inhibitor according to the present invention, in particular 4SC-202, and its respective salts, as well as of the at least one immune checkpoint modulator, in particular Pembrolizumab, are described in detail in the prior art, including the references cited herein.

In certain embodiments, two or more immune checkpoint modulators are combined in the treatment according to the present invention. For instance, the treatment may involve the administration of a first immune checkpoint modulator, aiming at a first immune checkpoint, e.g. an anti-PD-1 modulator, and the administration of a second immune checkpoint modulator, aiming at a second immune checkpoint, e.g. an anti-CTLA-4 modulator. In certain embodiments, two or more inhibitors of anti-inflammatory immune checkpoints, or two or more agonists of pro-inflammatory immune checkpoints are combined, or one or more inhibitors of anti-inflammatory immune checkpoints and one or more agonists of pro-inflammatory immune checkpoints are combined. In particular, two or three, more particularly two immune checkpoint modulators are combined. For instance, the combination of ipilimumab (anti-CTLA-4) plus nivolumab (anti-PD-1) has already been investigated.

This combination of two or more immune checkpoint modulators as described above can likewise apply in certain embodiments to the prior systemic treatment as detailed herein.

In certain embodiments of the present invention, the HDAC inhibitor and the at least one immune checkpoint modulator may be administered simultaneously, sequentially or separately. In particular embodiments the at least one immune checkpoint modulator (in case of an orally available, e.g. a small molecule) and/or the HDAC inhibitor as described herein, in particular the HDAC inhibitor, is administered to the patient having said cancer in the non-fasted state. Non-fasted state (or fed state) means in particular that the patient is not fasted. Particularly this means that active agents are administered together with or after food intake, e.g. a meal, e.g. breakfast, lunch and/or dinner, more particularly together with or after breakfast and/or dinner. In particular, the HDAC inhibitor (and optionally an orally available, e.g. a small molecule, immune checkpoint modulator) is administered within 5 hours, more particularly within 4 hours, even more particularly within 3 hours, yet even more particularly within 2.5 hours, yet even more particularly within 2 hours yet even more particularly within 60 minutes, yet even more particularly within 30 minutes after food intake, particularly a meal, more particularly a light meal, in particular a light breakfast and/or dinner. A recommendation by the physician for a light meal could be a small to medium portion of 1 or 2 of the following: fruit, yogurt, cereal, toast, croissant, bread, cold meat, cheese, or salad, with coffee, tea, juice, or milk or similar amounts of food, which typically amounts to around 150-300 kcal, while however the numbers and amounts may vary to some extent in the actual treatment setting, taking into account that the patients are typically treated ambulatory and thus for the most part determine their diet themselves.

In the case of twice daily administration of the HDAC inhibitor, the first administration may be within the time frames in relation to a breakfast or light breakfast as detailed above and particularly within 30 minutes after said breakfast or light breakfast; and the second administration may be within the time frames in relation to a dinner or light dinner as detailed above and particularly within 30 minutes after said dinner or light dinner. In the case of the immune checkpoint modulator being a biological, the administration typically does not have to be timed with respect to food intake, since the form of its application is typically parenteral. For the sake of completeness, food intake, meal, etc. logically refer to food intake, meal, etc. of the patient receiving the administration of the aforementioned agents.

In the further context of the present invention, the term “active agents” refers to a pharmaceutical agent exerting a medical effect on a disease or medical condition (e.g. an amelioration thereof) and said term in particular includes the HDAC inhibitor and the at least one immune checkpoint modulator, such as 4SC-202 and pembrolizumab.

In the embodiments of the present invention, the active agents may be provided in pharmaceutical compositions comprising one or more of said active agents and a pharmaceutically acceptable carrier or diluent. In particular, the HDAC inhibitor and the at least one immune checkpoint modulator may be provided in the same pharmaceutical composition (also known as a fixed combination), e.g. in the case of an orally available, e.g. a small molecule, immune checkpoint modulators or in separate pharmaceutical compositions (e.g. in two separate tablets, or in a tablet and a vial of powder, respectively).

Such pharmaceutical compositions may be provided in the context of pharmaceutical products, comprising e.g. one or more pharmaceutical compositions and packaging material. Said packaging material typically comprises a label or package insert which indicates that the active agent(s) is/are useful for treating the diseases detailed herein. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the active agents are either employed as such, or particularly in combination with suitable pharmaceutical auxiliaries and/or excipients, e.g. in the form of tablets, coated tablets, capsules, caplets, suppositories, patches (e.g. as TTS), emulsions, suspensions, gels or solutions, the active agent content advantageously being between 0.1 and 95% and where, by the appropriate choice of the auxiliaries and/or excipients, a pharmaceutical administration form (e.g. a delayed release form or an enteric form) exactly suited to the active agent and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other excipients, for example antioxidants, dispersants, emulsifiers, preservatives, solubilizers, colorants, complexing agents or permeation promoters, can be used.

In practicing the present invention and depending on the details, characteristics or purposes of their uses mentioned above, the active agents according to the present invention may be administered in combination therapy separately, sequentially, simultaneously or chronologically staggered (e.g. as combined unit dosage forms (in the case of the at least one immune checkpoint modulator being orally available, e.g. small molecules)), as separate unit dosage forms or adjacent discrete unit dosage forms, as fixed (in the case of the at least one immune checkpoint modulator being orally available, e.g. small molecules) or non-fixed combinations, as kit-of-parts or as admixtures (in the case of the at least one immune checkpoint modulator being orally available, e.g. small molecules)).

A “fixed combination” is defined as a combination wherein a first active ingredient and at least one further active ingredient are present together in one unit dosage or in a single entity (in the case of the at least one immune checkpoint modulator being orally available, e.g. small molecules). One example of a “fixed combination” is a pharmaceutical composition wherein the said first active ingredient and said further active ingredient are present in admixture for simultaneous administration, such as in a single formulation. Another example of a “fixed combination” is a pharmaceutical combination wherein the said first active ingredient and the said further active ingredient are present in one unit without being in admixture.

A “kit-of-parts” is defined as a combination wherein the said first active ingredient and the said further active ingredient are present in more than one unit. One example of a “kit-of-parts” is a combination wherein the said first active ingredient and the said further active ingredient are present separately. The components of the kit-of-parts may be administered separately, sequentially, simultaneously or chronologically staggered.

The first and further active ingredient of a combination or kit-of-parts according to this invention may be provided as separate formulations (i.e. independently of one another), which are subsequently brought together for simultaneous, sequential, separate or chronologically staggered use in combination therapy; or packaged and presented together as separate components of a combination pack for simultaneous, sequential, separate or chronologically staggered use in combination therapy.

The type of pharmaceutical formulation of the first and further active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. parenteral, in particular intravenous administration.

A further aspect of the present invention is a combination comprising, in non-fixed form, the HDAC inhibitor and one or more further therapeutic agents for sequential, separate, simultaneous or chronologically staggered use in therapy in any order. Optionally said combination comprises instructions for its use in therapy.

A further aspect of the present invention is a combined preparation, such as e.g. a kit of parts, comprising a preparation of the HDAC inhibitor and a pharmaceutically acceptable carrier or diluent and one or more further therapeutic agents; and optionally instructions for simultaneous, sequential, separate or chronologically staggered use in therapy.

A further aspect of the present invention is a kit of parts comprising a dosage unit of the HDAC inhibitor, a dosage unit of one or more further therapeutic agents, and optionally instructions for simultaneous, sequential or separate use in therapy.

A further aspect of the present invention is a pharmaceutical product comprising the HDAC inhibitor, or one or more pharmaceutical compositions comprising said compounds; and one or more further therapeutic agents, or one or more pharmaceutical compositions comprising said therapeutic agents, for simultaneous, sequential or separate use in therapy. Optionally this pharmaceutical product comprises instructions for use in said therapy.

A further aspect of the present invention is a pharmaceutical composition as unitary dosage form comprising, in admixture, the HDAC inhibitor one or more further therapeutic agents and optionally a pharmacologically acceptable carrier, diluent or excipient.

A further aspect of the present invention is a commercial package comprising the HDAC inhibitor together with instructions for simultaneous, sequential or separate use with one or more further therapeutic agents.

In addition, the combination according to the present invention can be used in the pre- or post-surgical treatment.

In further addition, the combination according to the present invention can be used in combination with radiation therapy, in particular in sensitization of patients towards standard radiation therapy.

The administration of the combination according to the present invention and pharmaceutical compositions according to the invention may be performed in any of the generally accepted modes of administration available in the art. Illustrative examples of suitable modes of administration include intravenous, oral, nasal, parenteral, topical, transdermal and rectal delivery. In a particular embodiment of the present invention, the administration of the HDAC inhibitor is via oral delivery and the administration of the at least one immune checkpoint modulator is parenteral, in particular intravenous in the case of a biological, via oral delivery, e.g. in the case of a small molecule.

In the embodiments of the present invention, doses refer to the amount of compound with respect to the free form of said compound, i.e. the free acid or free base form of said compound. Consequently, adducts, salts, etc. of such free acid or free base form are actually to be administered in a correspondingly higher dose in order to account for the weight of the counter-ion or adduct partner. For example, in relation to 4SC-202 tosylate salt, a “dose of 100 mg 4SC-202” relates to (rounded) 138 mg 4SC-202 tosylate salt—comprising 100 mg 4SC-202 free base and 38 mg toluenesulfonic acid (molecular weight of 4SC-202=447.513 g/mol; molecular weight of 4SC-202 tosylate salt=619.711 g/mol; therefore 100:447.513*619.711=138).

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicit, implicit or inherent disclosure) of the present invention without departing from the spirit and scope of this invention as defined by the scope of the appended claims.

As used herein, the term including, unless specified otherwise, is to be understood to mean “including, but not limited to”.

As used herein, expressions such as “is/are administered” likewise refer to “is/are to be administered”.

In the present invention, the administration of active agents may follow a certain schedule, which may include periods of daily administration of active agents and periods wherein only one of the active agents or no active agents are administered. Particularly, such a schedule consists of treatment cycles, wherein typically such treatment cycles can be repeated as often as necessary, i.e. as seen fit by the physician responsible for the treatment.

Treatment cycles are particularly 12-16 day treatment cycles, 2 week treatment cycles, 17-25 day treatment cycles, 3 week treatment cycles, 24-32 day treatment cycles, 4 week treatment cycles, 30-40 day treatment cycles, 5 week treatment cycles, 37-47 day treatment cycles, or 6 week treatment cycles, more particularly 2 week, 3 week, 4 week, 5 week, or 6 week treatment cycles, even more particularly 2 week, 3 week, or 4 week treatment cycles, yet even more particularly 2 week or 3 week treatment cycles, yet even more particularly 3 week treatment cycles.

Typically, in the case of the immune checkpoint modulator(s) being biologicals, they are administered only on day one of each treatment cycle. The treatment cycle then follows the usual administration cycle of the immune checkpoint modulator(s), e.g. as typically applied by physicians and/or as approved by the governmental authorities. In the case of the immune checkpoint modulator(s) being orally available, e.g. small molecules, they typically can be administered following the administration schedule of the HDAC inhibitor or continuously, or following a different pattern, e.g. a different pattern of the ones described herein for the HDAC inhibitor (e.g. the HDAC inhibitor being dosed every other day and the orally available, e.g. small molecule, checkpoint modulator(s) being administered daily.

Particularly, in the treatment cycles the HDAC inhibitor is administered daily (or alternatively every other day) in a dosing period of the first 4-10 days, more particularly the first 6-8 days in a 12-16 day treatment cycle; in a dosing period of the first 5-10 days, more particularly the first 6-8 days, even more particularly the first 7 days in a 2 week treatment cycle; in a dosing period of the first 10-18 days, more particularly the first 12-16 days, more particularly the first 13-15 days in a 17-25 day treatment cycle, in a dosing period of the first 10-18 days, more particularly the first 12-16 days, more particularly the first 13-15 days, yet even more particularly the first 14 days in a 3 week treatment cycle; in a dosing period of the first 18-28 days, more particularly the first 20-26 days, more particularly the first 22-25 days in a 24-32 day treatment cycle; in a dosing period of the first 18-24 days, more particularly the first 20-22 days, even more particularly the first 21 days in a 4 week treatment cycle; in a dosing period equal to two consecutive dosing periods described above for the 2 week treatment cycle in a 4 week treatment cycle; in a dosing period of particularly the first 25-35 days, more particularly the first 26-34 days, even more particularly the first 28-33 days in a 30-40 day treatment cycle; in a dosing period of the first 24-32 days, more particularly the first 26-30 days, even more particularly the first 28 days in a 5 week treatment cycle; in a dosing period of the first 30-43 days, more particularly in a dosing period of the first 32-42 days, even more particularly the first 35-40 days in a 37-47 day treatment cycle; in a dosing period equal to two consecutive dosing periods described above for the 3 week treatment cycle in a 6 week treatment cycle.

In certain embodiments, the aforementioned dosing periods, in particular dosing periods of 14 or more days, can be split into periods of 5-7 days of dosing, particularly 5 days of dosing, each followed by 1-3 days without dosing, particularly 1-2 days, more particularly 2 days without dosing. As an example, a 21 day dosing period in a 4 week treatment cycle can be split into three equal periods of 5 days dosing and 2 days without dosing, and a 14 day dosing period in a 3 week treatment cycle can be split into two equal periods of 5 days dosing and 2 days without dosing. “Dosing” refers to administration of the agent.

Alternatively, the HDAC inhibitor could be administered daily throughout the aforementioned treatment cycles, particularly at lower doses, e.g. at 75% or less, more particularly at 50% or less, even more particularly at 40% or less, yet even more particularly at 30% or less, yet even more particularly at 25% or less, of the MTD determined for the HDAC inhibitor in single treatment (i.e. for administration of the HDAC inhibitor alone). For instance, 4SC-202 might be administered daily throughout the aforementioned treatment cycles in a dose of 100-200 mg/day, or particularly about 100 mg/day, or in alternative embodiments in the daily doses detailed herein above.

In particular embodiments the treatment may involve repeating treatment cycles of e.g. 1) a dosing period of 8-20 days, wherein the at least one immune checkpoint modulator is administered on day one, and the HDAC inhibitor is administered daily, followed by 2) a rest period of 4-10 days wherein no active agent is administered (totaling a 12-30 day treatment cycle); or 1) a dosing period of 12-18 days, wherein the at least one immune checkpoint modulator is administered on day one, and the HDAC inhibitor is administered daily, followed by 2) a rest period of 5-8 days wherein no active agent is administered (totaling a 17-26 day treatment cycle); or 1) a dosing period of 14 days, wherein the at least one immune checkpoint modulator is administered on day one and the HDAC inhibitor is administered daily, followed by 2) a rest period of 7 days wherein no active agent is administered (totaling a 21 day treatment cycle); or 1) a dosing period of 14 days, wherein the at least one immune checkpoint modulator is administered on day one, and the HDAC inhibitor is administered on an alternating days basis (i.e. on day 1, 3, 5, 7, 9, 11, and 13), followed by 2) a rest period of 7 days wherein no active agent is administered (totaling a 21 day treatment cycle); or 1) a dosing period of 8-20 days, wherein the at least one immune checkpoint modulator is administered on day one, and the HDAC inhibitor is administered in two treatment cycles of 3-7 days daily administration followed by 1-4 days wherein the HDAC inhibitor is not administered, followed by 2) a rest period of 4-10 days wherein no active agent is administered (totaling a 12-30 day treatment cycle); or 1) a dosing period of 12-18 days, wherein the at least one immune checkpoint modulator is administered on day one, and the HDAC inhibitor is administered in two treatment cycles of 4-6 days daily administration followed by 2-3 days wherein the HDAC inhibitor is not administered, followed by 2) a rest period of 5-8 days wherein no active agent is administered (totaling a 17-26 day treatment cycle); or 1) a dosing period of 14 days, wherein the at least one immune checkpoint modulator is administered on day one, and the HDAC inhibitor is administered in two treatment cycles of 5 days daily administration followed by 2 days wherein the HDAC inhibitor is not administered, followed by 2) a rest period of 7 days wherein no active agent is administered (totaling a 21 day treatment cycle).

Said respective latter schemes, in particular the respective 21-day treatment cycle regimes provide for a particularly desirable tolerability while still enabling for therapeutic benefits and patient compliance.

In other particular embodiments the treatment may involve repeating treatment cycles of 1) a dosing period of 7-10, particularly 7 days, wherein the at least one immune checkpoint modulator is administered on day one and the HDAC inhibitor is administered daily, followed by 2) a rest period of 4-7, particularly 7 days wherein no active agent is administered (totaling a 14 day treatment cycle); or 1) a dosing period of 7-10, particularly 7 days, wherein the at least one immune checkpoint modulator is administered on day one, and the HDAC inhibitor is administered on an alternating days basis (i.e. on day 1, 3, 5, 7 (and 9)), followed by 2) a rest period of 4-7, particularly 7 days wherein no active agent is administered (totaling a 14 day treatment cycle).

In other particular embodiments the treatment may involve repeating treatment cycles of 1) a dosing period of 21 days, wherein the at least one immune checkpoint modulator is administered on day one, and the HDAC inhibitor is administered daily, followed by 2) a rest period of 7 days wherein no active agent is administered (totaling a 28 day treatment cycle); or 1) a dosing period of 21 days, wherein the at least one immune checkpoint modulator is administered on day one and the HDAC inhibitor is administered on days 1-5, 8-12 and 15-19, followed by 2) a rest period of 7 days wherein no active agent is administered (totaling a 28 day treatment cycle); or 1) a dosing period of 21 days, wherein the at least one immune checkpoint modulator is administered on day one, and the HDAC inhibitor is administered on an alternating days basis (i.e. on day 1, 3, 5, 7, 9, 11, 13, 15, 17, 19 and 21), followed by 2) a rest period of 7 days wherein no active agent is administered (totaling a 28 day treatment cycle).

In particular embodiments the treatment may involve a first treatment cycle, wherein only the HDAC inhibitor in administered, (i.e. no immune checkpoint modulator is administered). Said first treatment cycle (which can also be called a priming cycle) is then followed by the treatment with the combination of the at least one immune checkpoint modulator and the HDAC inhibitor as detailed herein, in particular in the form of the treatment cycles as described above. For instance, said first treatment cycle may involve 1, 2, 3, or 4 weeks of daily administration, or alternatively administration on an alternating days basis, of the HDAC inhibitor, optionally followed by a period, in particular one week, wherein no drug is administered. Said treatment cycle may otherwise (except for the administration of the immune checkpoint modulator) be equal in duration and dosing of the HDAC inhibitor to the treatment cycles described above. In particular embodiments, the HDAC inhibitor is 4SC-202 and is administered in a dose of 150 to 250, particularly 175 to 225, more particularly 190 to 210, even more particularly 195 to 205, yet even more particularly about 200 mg/day,

or, in alternative particular embodiments, the HDAC inhibitor is 4SC-202 and is administered in a dose of 80 to 120, particularly 90 to 110, more particularly 95 to 105, even more particularly about 100 mg/day,

or, in other alternative particular embodiments, the HDAC inhibitor is 4SC-202 and is administered in a dose of 300 to 500, particularly 350 to 450, more particularly 375 to 425, even more particularly 390 to 410, yet even more particularly 395 to 405, yet even more particularly about 400 mg/day,

wherein the aforementioned daily dose may be administered in two portions (each half of the aforementioned amounts), twice daily, particularly one each in the morning and evening (wherein particularly the evening dose is administered 10-14, more particularly 11-13, even more particularly about 12 hours after the morning dose).

Said first treatment cycle may prime the patients' immune system for the immune checkpoint modulator (e.g. by increasing infiltration of immune cells into the tumor, or by increasing activity of the immune cells in the tumor—which may be determined e.g. via immune histochemistry, methods of which are well known in the field, such as for example the methods described in Arpita Kabiraj et al., Int J Biol Med Res. 2015; 6(3): 5204-5210 and references therein to the specific methods) and may thus lead to better outcome, tolerability, and or shorter duration of treatment with the immune checkpoint modulator. Particularly, said first treatment cycle increases the number of immune cells and/or the ratio of immune cells versus total cell number in said tumor, in particular in the core (CT) and/or the invasive margin (IM) of the said tumor, more particularly by at least 10%, even more particularly by at least 20%, yet even more particularly by at least 50%, yet even more particularly by at least 100%, yet even more particularly by at least a factor of 3, yet even more particularly by at least a factor of 5, yet even more particularly by at least a factor of 10. Said immune cells are particularly t-cells, more particularly CD3+ and/or CD8+ cells.

Said treatment cycle may involve the same schedule of HDAC inhibitor administration as the following treatment cycles (with respect to timing and dosage). Alternatively, said treatment cycle may involve a different schedule of HDAC inhibitor administration than the following treatment cycles (with respect to timing and/or dosage). In a certain embodiment, the dose of HDAC inhibitor administered in said first treatment cycle is optionally higher than the dose of HDAC inhibitor administered in said following treatment cycles (e.g. 50% or more, 100% or more, 200% or more), and/or the frequency of HDAC inhibitor administration is higher than in said following treatment cycles (e.g. daily administration in the first treatment cycle, administration every other day in the following treatment cycles), and/or the periods of consecutive daily administration of HDAC inhibitor in said first treatment is longer than in said following treatment cycles (e.g. a dosing period of 1-3, particularly 2-3, more particularly 2 weeks wherein the HDAC inhibitor is administered daily in the first treatment cycle, and a dosing period of 1-3, particularly 2-3, more particularly 2 weeks wherein the HDAC inhibitor is administered in 1-3, particularly 2-3, more particularly 2 treatment cycles of 5 days daily administration followed by 2 days wherein the HDAC inhibitor is not administered in the following treatment cycles). The reduction of HDAC inhibitor administration (dose, duration, frequency) in the following cycles may improve tolerability of the HDAC inhibitor/immune checkpoint modulator combination treatment.

In a particular embodiment of the first treatment cycle, the HDAC inhibitor is administered daily for 14 or 7 days, more particularly 14 days, followed by 7 days wherein no active agent is administered.

In particular embodiments, on dosing days of the at least one immune checkpoint modulator, in particular Pembrolizumab, the HDAC inhibitor, particularly 4SC-202 is to be administered after the administration of the at least one immune checkpoint modulator.

References and claims to the use of a certain compound for the manufacture of a medicament for the treatment of cancer in to be used combination with a certain second agent in their general and specific forms likewise relate to the use of said compound for the manufacture of a medicament for the treatment of cancer in combination with said second agent; to methods of treating said disease or medical condition, said method comprising administering a therapeutically effective and tolerable amount of said certain compound to a subject in need thereof, and administering a therapeutically effective and tolerable amount of said second agent to said subject; to methods of treating said disease or medical condition, said method comprising administering a therapeutically effective and tolerable amount of said certain compound to a subject in need thereof, said certain compound to be used in combination with said second agent; to compositions comprising said certain compound for the treatment of said disease or medical condition in combination with said second agent; to compositions comprising said certain compound for the treatment of said disease or medical condition, said composition to be used in combination with said second agent; to said certain compound for use in the treatment of said disease or medical condition in combination with said second agent; said certain compound for use in the treatment of said disease or medical condition to be used in combination with said second agent; and vice versa.

### Examples

The following examples serve to illustrate the invention further without restricting it.

A) Preclinical Murine In Vivo Study

In vivo murine tumor models were conducted with a combination of 4SC-202 and murine anti-PD-1 and anti-PD-L1 antibodies, respectively. 1×10⁶ CT26 cells or 30-50 mg C38 tumor fragments (both human colon carcinoma cell lines) were subcutaneous implantation into the right flank of female C57Bl/6 (C38 tumor fragments) or Balb/c (CT26 cells) mice, respectively, at day 0 of the experiment.

Protocol C38: At D10, when tumors reached a mean volume of 142±27 mm³, 160 mice out of 224, were randomized according to their individual tumor volume into 8 groups of 20 mice each.

The treatments were initiated at D11 as follows:
Animals from group 1 received twice daily PO administrations of vehicle for 32.5 consecutive days from D11 to D43 (2Q1 Dx32.5),
Animals from group 2 received one daily PO administration of 4SC-202 at 20 mg/kg/adm for 33 consecutive days from D11 to D43 (Q1 Dx33),
Animals from group 3 received one IP injection of anti-PD1 at 10 mg/kg twice weekly for two consecutive weeks (at D11, D14, D17 and D21: TWx2),
Animals from group 4 received one daily PO administration of 4SC-202 at 20 mg/kg/adm for 33 consecutive days from D11 to D43 (Q1 Dx33) in combination with one IP injection of anti-PD1 at 10 mg/kg twice weekly for two consecutive weeks (at D11, D14, D17 and D21: TWx2),
Animals from group 5 received twice daily PO administrations of 4SC-202 at 20 mg/kg/adm 32.5 consecutive days from D11 to D43 (2Q1 Dx32.5),
Animals from group 6 received twice daily PO administrations of 4SC-202 at 20 mg/kg/adm 32.5 consecutive days from D11 to D43 (2Q1 Dx32.5) in combination with one IP injection of anti-PD1 at 10 mg/kg twice weekly for two consecutive weeks (at D11, D14, D17 and D21: TWx2),
Animals from group 7 received one daily PO administration of 4SC-202 at 60 mg/kg/adm for 27 consecutive days from D11 to D37 (Q1 Dx27),
Animals from group 8 received one daily PO administration of 4SC-202 at 60 mg/kg/adm for 27 consecutive days from D11 to D37 (Q1 Dx27) in combination with one IP injection of anti-PD1 at 10 mg/kg twice weekly for two consecutive weeks (at D11, D14, D17 and D21: TWx2).

Protocol CT26: At D7, when tumors reached a mean volume of 73±21 mm³, 80 mice out of 104, were randomized according to their individual tumor volume into 4 groups of 20 mice each. The treatments were initiated at D8 as follows:
Animals from group 1 received twice daily PO administrations of vehicle for 16.5 consecutive days from D8 to D24 (2Q1 Dx16.5),
Animals from group 2 received one IP injection of anti-PD-L1 at 10 mg/kg every two other days for 8 injections (at D8, D10, D12, D14, D16, D18, D20 and D22: Q2Dx8),
Animals from group 3 received one daily PO administration of 4SC-202 at 20 mg/kg/adm for 19.5 consecutive days from D8 to D27 (2Q1 Dx19.5),
Animals from group 4 received twice daily PO administrations of 4SC-202 at 20 mg/kg/adm for 33 consecutive days from D8 to D40 (2Q1Dx33) in combination with one IP injection of anti-PD-L1 at 10 mg/kg every two other days for 8 injections (at D8, D10, D12, D14, D16, D18, D20 and D22: Q2Dx8).

For both groups (C38 and CT26) 4SC-202 Tosylate was suspended in 2% methocel solution at 2 or 6 mg/ml (above administered concentrations refer to the calculation for 4SC-202 free base). 4SC-202 was administered by oral gavage (per os, PO) via a gavage tube. Anti-PD-1 or anti PD-L1 antibody was diluted with PBS in order to reach final concentration of 1 mg/ml. Animal housing and experimental procedures were realized according to the French and European Regulations and NRC Guide for the Care and Use of Laboratory Animals.

Monitoring of mice body weights and tumor volume twice a week. Monitoring of viability and behaviour every day. Tumor width and length were measured using a digital caliper twice a week and estimated tumor volumes were calculated from measured tumor length and width using the formula Estimated tumor volume=width²×length/2. Experiments and measurements were either terminated at indicated days (tumor growth graphs) or continued until animals had to be sacrificed to monitor their survival (Kaplan-Meier plots).

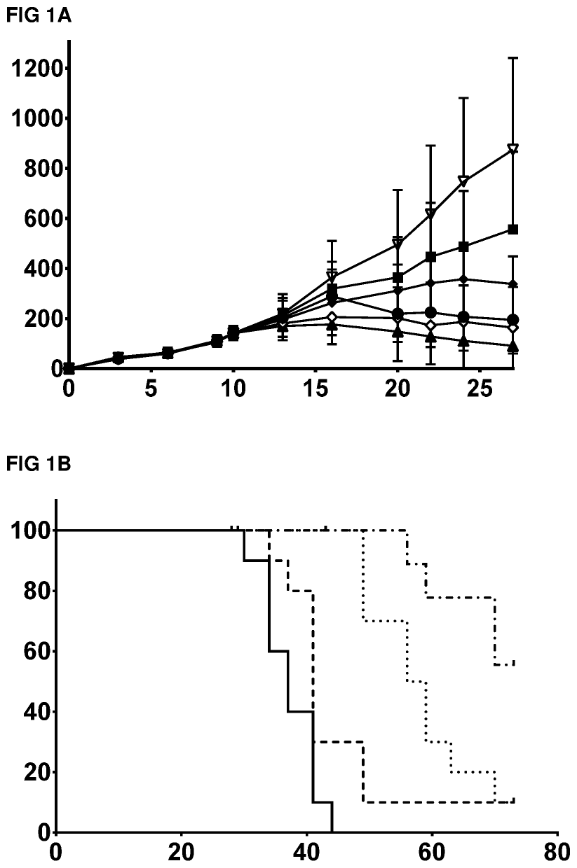

Results are shown in FIGS. 1 (CD38) and 2 (CT26). No significant anti-tumor activity was observed for mice treated with antibody when compared to mice treated with vehicle. In contrast, an increase of the antitumor activity was observed for mice treated with 4SC-202 at 20 mg/kg alone, and for the combination. This demonstrates that reduction of tumor growth and survival rate are improved in particular with the combination of 4SC-202 and the immune checkpoint modulator.

B) Food Effect—Dog Study

A pharmacokinetic profile for 4SC-202 was determined in an animal study comprising three dogs (male Beagle). To allow for better comparison of the fasted/fed values and eliminate inter-subject variability, each dog first received a first dose of 4SC-202 in the fasted state, and after a wash-out phase of one week, received another dose of 4SC-202 in the fed state. After each respective dose, the plasma pK of 4SC-202 was determined

Before the first dosing, the dogs were fasted overnight and food was returned after the 4 h post-dose blood sampling collections. On each dosing day, the dogs received a single intramuscular injection of freshly prepared Pentagastrin (6 μg/kg and 0.05 mL/kg) 30-33 min prior to oral treatment with approx. 60 mg/kg 4SC-202 (tablet as detailed herein below); for fed animals, these steps were omitted and the dogs received their usual meal approx. 30 min prior to dosing. The number of tablets (4SC-202 tosylate corresponding to 100 mg 4SC-202 free base per tablet) was rounded to the nearest whole tablet based on dose and animal pre-dose body weight. Immediately after dosing the animals received 20 mL of water after oral gavage or were offered 20 mL of water to assist swallowing of the tablets.

Blood samples (1 ml per sample in K₂EDTA as anticoagulant) were collected by venipuncture of a peripheral vein and placed on wet ice immediately after collection until centrifugation as 2000*g for 10 min at 2-8° C. to isolate plasma. The resulting plasma was transferred into individual polypropylene tubes in a 96-well plate format and immediately placed on dry ice until storage at −20° C. before measurement. Blood samples were collected from each animal prior to dosing, and at 0.5, 1, 1.5, 2, 2.5, 3, 4, 6, 10, 12, and 24 h following oral dosing.

The concentration of 4SC-202 (free base) in plasma samples was determined by first precipitating the samples with three volume equivalents of acetonitrile containing the internal standard. After filtration using a 96-well protein precipitation plate, an aliquot of 2 μL of the sample was injected onto the HPLC system. The calibration applied was 1.00 to 1000 ng/mL 4SC-202 (free base) in human plasma (K3-EDTA).

Instruments:

HPLC pump—1200 series binary pumps—Agilent Technologies Inc, Santa Clara, Calif., USA; Autosampler—CTC PAL—CTC Analytics AG, Zwingen, Switzerland; Mass spectrometer—TSQ Vantage—Thermo Fisher Scientific, San Jose, Calif., USA; Liquid handling—Freedom Evo—Tecan, Mannedorf, Switzerland. Software: LCquan 2.5.6—Thermo Fisher Scientific, San Jose, Calif., USA; Xcalibur 2.0.7—ThermoFisher Scientific, San Jose, Calif., USA; Evoware 2.3—Tecan, Mannedorf, Switzerland

Preparation of Calibration Samples and Quality Control Samples:

For the preparation of calibration samples, 4SC-202 tosylate salt was dissolved in dimethylsulfoxide to a concentration of 1.00 mg/mL free base. For the preparation of QC samples, a second solution with a concentration of 1.00 mg/mL free base was used. Working solutions were prepared by serial dilutions in a mixture of dimethylsulfoxide/acetonitrile (50/50, v/v) to concentrations 50 times higher than the corresponding concentrations in matrix. The concentrations were calculated under consideration of purity and salt factor where applicable. All concentrations are expressed as those of the free base. A light instability was observed and solutions were handled under light protected conditions. The concentrations of the calibration are (ng/mL): 1.00, 2.00, 5.00, 20.00, 100, 500, 750, 1000. The concentrations of the QC are (ng/mL): 13.00, 20.00, 750 For preparation of the CAL and QC, the matrix was fortified with the working solutions using a ratio of 98/2 (v/v). For the preparation of the internal standard solution, 4SC-201-d6 mesylate salt was dissolved in dimethylsulfoxide to a concentration of 1.00 mg/mL of the free base. Further dilutions to a final concentration of 100 ng/mL were done in acetonitrile. The concentration was calculated under consideration of purity and salt factor.

Sample Preparation:

An aliquot of 150 μL of the internal standard solution was transferred to a 96-well protein precipitation plate and an aliquot of 50 μL human plasma were added. After mixing (5 minutes at 700 rpm), a slight vacuum was applied for the filtration step into a deep well plate. Note: In case of limited sample volume where an automated sample processing will not be possible the respective sample was processed by manual pipetting. The volume ratios were kept constant. After protein precipitation the samples were centrifuged for about 10 minutes at approximately 50000 g. The temperature of the centrifuge was set to 8° C. An aliquot of the supernatant was transferred to the deep well plate.

LC-MS/MS conditions: The analysis was done by separation using reversed phase chromatography followed by detection with triple-stage quadrupole MSIMS in the selected reaction monitoring mode. LC: Mobile Phase A—0.1% aqueous acetic acid; Mobile Phase B methanol containing 0.1% acetic acid; Column: YMC Pro C4, 2.1×50 mm, 5 μm (YMC Co. Ltd., Kyoto, Japan); Injection volume 2 μL (10 μL in a 2 μL sample loop); Column temperature 40° C.

Gradient:

| Time | Flow | Phase A | Phase B |
|---|---|---|---|
| [min] | [mL/min] | [%] | [%] |
| 0.00 | 0.700 | 100.0 | 0.0 |
| 0.50 | 0.700 | 100.0 | 0.0 |
| 2.50 | 0.700 | 5.0 | 95.0 |
| 4.50 | 0.700 | 5.0 | 95.0 |
| 4.51 | 0.700 | 100.0 | 0.0 |
| 5.01 | 0.700 | 100.0 | 0.0 |

Mass Spectrometry:

Ion source: HESI; Polarity: Positive; Voltage [V]: 2500; Sheath gas [au]: 60; Sweep gas [au]: 0; Auxiliary gas [au]: 5; Vaporizer [° C.]: 350; Capillary temperature [° C.]: 350; Collision gas pressure [mTorr]: 1.0 to 1.5. Autosampler: Wash 1—ethanol/water (1/1, v/v); Wash 2—acetonitrile/methanol/2-propanol (1/1/1, v/v/v); Tray temperature—8±5° C.

Data Acquisition and Processing:

The concentration of the analyte was calculated using the internal standardization method. The area ratio of analyte to internal standard against the concentration of calibration samples were used for quantification. The acquisition and processing of data results was performed using LCquan 2.5.6 and Xcalibur 2.0.7. Microsoft Office Excel was used for calculation and statistical evaluation of CAL and QC concentration data. For 4SC-202 (free base) the fitting of data was done with a weighting factor of 1/x² using a linear regression with the method of least squares.

Pharmacokinetic parameters were derived by non-compartmental pharmacokinetic analysis using WinNonlin Version 7.0 or higher.

Tablet:

4SC-202 tosylate 138.4 mg; microcrystalline cellulose 308.8 mg; calcium carbonate 308.8 mg; sodium croscarmellose 24.0 mg; magnesium stearate 12.0 mg; colloidal anhydrous silica 8.0 mg.

Dosing Scheme for First and Second Dose

| | Weight | No. of | Administered | Administered |
|---|---|---|---|---|
| Dog No. | (kg) | tablets | dose (mg) | dose (mg/kg) |
| 1 | 8.60 | 5 | 500 | 58.1 |
| 2 | 10.60 | 6 | 600 | 56.6 |
| 3 | 8.40 | 5 | 500 | 59.5 |

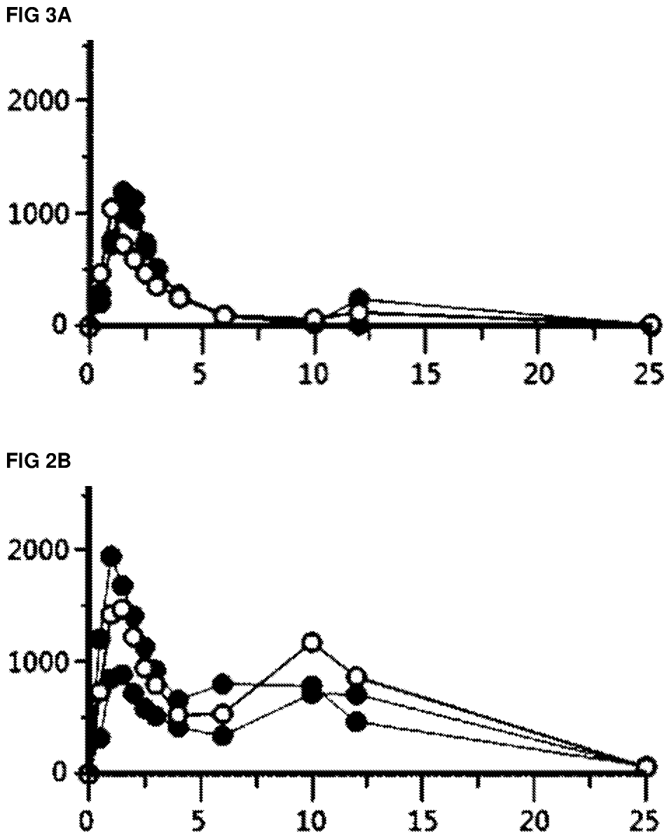

Results are shown in FIG. 3 and in the following table:

| | AUC geometric mean | CV(%) geometric mean |
|---|---|---|
| fasted | 3980.69 | 24.15 |
| fed | 13944.67 | 17.32 |

CV % = coefficient of variation

The AUC increase of the fed state is about 3.5-fold over the fasted state.

C) Clinical Study

The study is a Phase Ib/II open-label, multi-center study with a priming cycle of 4SC-202 to evaluate the safety, tolerability and preliminary efficacy of combination treatment with 4SC-202 and Pembrolizumab. A dose expansion cohort at the Recommended Phase Two Dose (RPTD) is added. The dose escalation phase follows a modified “rolling six” design. Cohorts of 10 patients receive pre-defined escalating doses of 4SC-202 in combination with Pembrolizumab. Subjects are patients with unresectable stage III or stage IV cutaneous melanoma, as per AJCC (Version 8) staging system (must have been histologically confirmed at least once during course of disease) and patients with metastatic tumor of unknown primary site and histology of melanoma.

Treatment consists of a first treatment cycle of 4SC-202 monotherapy (Cycle 1) at the dose of the current dose level followed by further cycles of combination therapy of 4SC-202 and Pembrolizumab (Cycle 2 and following). The monotherapy cycle allows the epigenetic changes and immune-modulation in the tumor microenvironment to occur before initiation of Pembrolizumab. Patients are treated in cycles of 21 days. In each cycle, patients are dosed with 4SC-202 for 14 days on (day 1-14) and 7 days off (day 15-21) treatment. Starting from Cycle 2, patients receive Pembrolizumab each cycle on day 1 (i.e. Q3W, every 3 weeks) at a dose of 2 mg/kg bodyweight. Treatment continues for 34 cycles (102 weeks), until progression of disease, unacceptable toxicity or withdrawal of consent, whichever is earlier. Dose levels (DL) of 4SC-202 (during first treatment cycle only administration of 4SC-202 at the respective dose level): DL1: 100 mg once daily 4SC-202 p.o (14 day on/7 day off schedule)+2 mg/kg Pembrolizumab i.v.; DL2: 200 mg once daily 4SC-202 p.o (14 day on/7 day off schedule)+2 mg/kg Pembrolizumab i.v.; DL3: 200 mg twice daily 4SC-202 p.o (14 day on/7 day off schedule)+2 mg/kg Pembrolizumab i.v. 4SC-202 tablets are to be taken within 2 h after a light breakfast in the morning and in the case of BID application schedules, tablets are to be taken in the morning within 30 min after a light breakfast and in the evening within 30 min after dinner.

Tablet:

4SC-202 tosylate 138.4 mg; microcrystalline cellulose 308.8 mg; calcium carbonate 308.8 mg; sodium croscarmellose 24.0 mg; magnesium stearate 12.0 mg; colloidal anhydrous silica 8.0 mg. Pembrolizumab is administered at a fixed dose of 2 mg/kg body weight on Day 1 of Cycle 2 and following cycles. It is provided as vials of powder containing 50 mg of Pembrolizumab. After reconstitution with sterile deionized water (e.g. milliQ grade) or aqueous NaCl 0.9, or glucose 5% solution, 1 mL of concentrate will contain 25 mg of Pembrolizumab. Pembrolizumab will be administered intravenously over 30 minutes every Day 1 of a Cycle (starting with Cycle 2). An infusion volume of 100-200 ml aqueous NaCl 0.9, or glucose 5% solution is to be used.

Dose reductions in individual patients for 4SC-202 are possible depending on the occurrence of adverse events. No dose reductions for Pembrolizumab are allowed. Dose limiting toxicities are evaluated during the DLT period: the first 3 cycles, i.e. the 4SC-202 monotherapy cycle (cycle 1) and the following 2 cycles of 4SC-202 and Pemrolizumab combination therapy (cycle 2 and 3). Patients who receive at least 11 administrations of the full planned daily dose of 4SC-202 and at least 2 doses of Pembrolizumab, but who have not experienced any DLT, are considered non-evaluable for DLT assessment; these patients are replaced.

A non-tolerated dose level is a dose level with an observed incidence of patients with at least 1 DLT during the DLT period of: 2 in the first 3, 4, 5 or 6 patients; 3 in the first 7, 8 or 9 patients; 4 in the first 10 patients. The MTD is considered the dose level below the non-tolerated dose level.

The recommended phase 2 dose (RPTD) for the expansion cohort is defined after reaching the MTD or after completion of enrollment to the highest dose level, taking results of safety, efficacy, PD, PK and modulation of tumor microenvironment into account. If DL1 is considered non-tolerable, then the sponsor can evaluate a dose level of 4SC-202 100 mg QAD (every alternate day) for 14 days on and 7 days off treatment, in combination with Pembrolizumab 2 mg/kg i.v. Q3W.

Expansion:

Depending on the results of the evaluation of the data on safety, efficacy, PK and PD, an RPTD will be selected and further 10 patients are enrolled in an expansion cohort at the RPTD or other selected dose level. The dosing follows the same schedule as specified for dose escalation.

Duration of Treatment:

Patients are treated at the respective dose level of 4SC-202 and Pembrolizumab for a maximum of 102 weeks (or 34 cycles, whatever is earlier) or until (whatever is earlier): Non-acceptable toxicity effects persist despite toxicity measures, as per judgement of the investigator; Patient wishes to discontinue the treatment; If a DL is considered non-tolerable, patients may continue at their current dose level at the discretion of the investigator if the benefit/risk ratio is still considered positive for the individual patient and the patient has not experienced a DLT; If a patient has experienced a DLT, the patient may continue treatment at the next lower DL of 4SC-202 at the discretion of the investigator. Dose reductions of Pembrolizumab are not allowed; Progressive disease. Patients tolerating the study drug, showing signs of clinical benefit despite showing progressive disease (PD) at tumor assessment may continue treatment until next tumor assessment for confirmation of PD or as long as they show signs of clinical benefit as per judgment of investigator Follow-up for survival and adverse events occur for individual patients for a maximum of 122 weeks (±2 weeks) after first dose of study treatment.

After permanent discontinuation of both study treatments or after the 102 weeks/34 cycles treatment period (whichever is earlier), patients undergo the end of treatment visit (EOT) within 28 days after the last study drug administration and are subsequently followed-up for survival until 122 weeks after Cycle 1 Day 1. Last SAE/AE and pregnancy information are collected up to 20 weeks (±2 weeks) after the last study drug administration.

After the last follow-up for survival, 122 weeks (±2 weeks) after Cycle 1 Day 1, patients undergo end of study (EOS) where the last survival and anti-cancer information within the study is captured. During this contact, last information with regard to potential AEs is collected for patients who completed the 102 weeks treatment period.

Definition of DLTs

All toxicities are graded according to the National Cancer Institute (NCI)-CTCAE Version 4.03. Safety assessments are performed at each cycle. For escalation and MTD determination purposes, the occurrence of DLTs during the first 3 Cycles of treatment (DLT period from C1 D1 to C3D21) is relevant. A DLT is defined as any of the following conditions, considered to be related to any of the two study drugs:

Hematologic toxicities: Neutropenia (Grade 4 neutropenia lasting >7 days; Febrile neutropenia of any duration); Thrombocytopenia (Grade 3 thrombocytopenia of any duration with any sign of clinically significant or hemoglobin relevant bleeding; Grade 4 thrombocytopenia of any duration (with or without any signs of bleeding)); Anaemia (Grade 3 anemia lasting >7 days; o Grade 4 anemia of any duration). Non-hematologic toxicities: Any Grade 4 non-hematologic toxicity of any duration; A clinically relevant non-hematologic toxicity Grade 3 EXCEPT for Diarrhea, nausea or vomiting for no more than 3 days duration after initiation of adequate medical treatment; Rash of the acneiform, pustular or maculopapular type, which resolves to Grade ≤2 within 7 days of study drug interruption and initiation of adequate medical treatment (whichever is later)—a recurrence at the same dose level after resumption of study drug is considered a DLT; Hyperglycaemia that can be controlled with insulin and resolves to Grade ≤2 within 7 days of study drug interruption; Hypothyroidism that can be managed with replacement therapy; Hyperthyroidism that can be managed with symptomatic therapy and resolves to Grade ≤2 within 7 days of study drug interruption; Fatigue which resolves to grade ≤2 within 7 days of study drug interruption and does not recur at the same level with resumption of study drug at the same dose (with optimal medical management); Oral mucositis/stomatitis which resolves to Grade ≤2 within 7 days of study drug interruption and does not recur at the same level with resumption of study drug at the same dose (with optimal medical management); A non-hematologic clinical laboratory AE that is asymptomatic and rapidly reversible (returns to baseline or to ≤Grade 1 within 7 days). Treatment must be held in the face of such Grade 3 non-hematologic laboratory adverse events unless and until it is clear that the event is not causally related to study drugs. General: Inability to begin the next dosing of 4SC-202 within 14 days (counted relative to day 1 of the next scheduled cycle) due to unresolved toxicity; Inability to begin the next dosing of Pembrolizumab within 28 days (counted relative to day 1 of the next scheduled cycle) due to unresolved toxicity; Recurrent/persistent Grade 2 toxicities which in the judgement of the investigator or sponsor or both are dose limiting; Any study drug related toxicity necessitating dose reduction of 4SC-202 during Cycle 1-3. For the DLT relevant calculation of AE duration, the day of onset is counted as day 1, the end day is not counted.

Criteria for Evaluation

Primary Endpoint: Safety and tolerability of the combination of 4SC-202 and Pembrolizumab is assessed from adverse events, laboratory tests, vital signs, ECGs, ECOG PS, physical examination and assessment of concomitant medications. Adverse events are evaluated using the National Cancer Institute (NCI) Common Terminology Criteria for Adverse Events (CTCAE) Version 4.03 for grading.

Secondary Endpoints: Preliminary anti-tumor efficacy of combination treatment with 4SC-202 and Pembrolizumab in terms of response rates and survival when administered in combination is determined using irRECIST v1.1. Anti-tumor efficacy is assessed by calculating the following parameters: Objective Response Rate (ORR); Best Overall Response (BOR); Disease Control Rate (DCR); Duration of Response (DOR); Progression Free Survival (PFS); Time to Progression (TTP); Overall Survial (OS).

Exploratory Endpoints: Exploratory endpoints are measured by the following parameters: Extent of HDAC-related pharmacodynamic changes in peripheral blood pre- and post dosing; Quantification of immune cell infiltration using immunohistochemistry (IHC) of sequential biopsies to analyze the tumor and tumor microenvironment and dynamics in sequential biopsies pre- and post dosing (IHC methods are well known in the field, for a review see e.g. Arpita Kabiraj et al., Int J Biol Med Res. 2015; 6(3): 5204-5210 and references therein to the specific methods); Quantification of alterations in gene expression in the tumor and tumor microenvironment in sequential biopsies pre- and post dosing under treatment; Ribonucleic acid (RNA)-profiling in biopsies and peripheral blood pre- and post dosing; Relationship between systemic exposure of 4SC-202 and relevant metabolites and PD pharmacodynamic markers, immune related biomarkers and gene expression; Relationship between efficacy endpoints and systemic exposure of 4SC-202, immune related biomarkers and gene expression

Number of Patients In each dose cohort approximately 10 patients are included, with up to 3 dose cohorts. An expansion cohort of another 10 patients is added in the defined RPTD dose level cohort

Inclusion Criteria

All inclusion criteria must be met during screening, unless otherwise stated.

1. Male or female patients, age ≥18 years at screening date

2. ECOG PS 0-1

3. Patients with unresectable stage III or stage IV cutaneous melanoma, as per AJCC (Version 8) staging system (must have been histologically confirmed at least once during course of disease)

4. Patients with metastatic tumor of unknown primary site and histology of melanoma are eligible

5. Patients must be primary refractory or non-responding to anti-PD-1 monotherapy as last systemic cancer directed treatment consisting of at least 2 cycles The following definitions apply: I. Primary refractory: Patients not achieving a response of SD or better (i.e. not achieving disease control) during anti-PD-1 therapy or within 6 months after the last dose of such treatment II. Non-responding: Patients not having achieved a response (CR or PR), and not having achieved a stable disease of a duration of more than 6 months

6. Patients must have progressed during or after anti-PD-1 therapy

7. Patients must have had anti-PD-1 therapy as the last systemic cancer directed treatment and must have received the last anti-PD-1 administration within 6 months prior to screening

8. Patients must have been tested for BRAF V600 mutation status

9. Patients with BRAF-mutated melanoma must have had a BRAF-mutation directed therapy, unless they were not considered eligible (e.g. due to contraindications) to such treatment

10. Measurable disease by CT or MRI per irRECIST 1.1 criteria, with longest diameter for non-nodal lesions ≥10 mm and ≥15 mm in short axis for nodal lesions

11. At least one tumor site (either primary site or metastasis) must be accessible for sequential biopsies and patient must consent to the 3 mandatory biopsies. (The sponsor may waive this requirement in individual cases)

Exclusion Criteria:

1. Patients not consenting to use adequate contraception as required per protocol.

2. Patients currently participating or who have participated in a study of an investigational agent or who are using an investigational device or have done so within 28 days of the first dose of study drug. (A patient in the Survival Follow-up phase of an investigational agent where no further treatment is expected is eligible) Disease/patient related:

3. Patients who achieved, during or after prior anti-PD-1 therapy, a response of CR or PR; or SD of a duration of more than 6 months (counted from the first dose of prior anti-PD1 therapy to first date of documented progression)

4. Life expectancy below 3 months

5. Patients with uveal or mucosal melanoma

6. Patients with symptomatic brain metastases/CNS involvement

7. Patients with inadequate organ function, defined as: a) Absolute neutrophil count (ANC)<1500/μL b) Hemoglobin (Hb)<9 g/dL c) Platelet count <100,000/μL d) Potassium outside of normal limits and not correctable with supplements e) Serum creatinine >1.5×ULN or eGFR <50 mL/min (as per cockroft-gault formula). f) ALT and/or AST >2.5×ULN. g) Serum total bilirubin >1.5×ULN. h) LDH >5×ULN. Prior/concurrent treatment related:

8. Remaining relevant toxicity (excluding alopecia, fatigue) to previous therapy has not resolved to Grade 1 (applicable at screening)

9. Patients with a history of anti-PD-1/immune-related adverse drug reactions of Grade 4 or Grade 3 and a high risk of re-occurrence (as judged by the investigator)

10. Prior treatment with a HDAC or LSD1 inhibitor or both

11. Prior treatment with anti-PD-L1 or anti-PD-L2 agents (prior treatment with antiCTLA4-agents is allowed)

12. Patients with precedent systemic anti-cancer therapy including chemotherapy, endocrine therapy, immunotherapy or use of other investigational agents prior first study drug administration. (A wash-out period of 5 half-lives or 4 weeks (whatever is shorter) must be respected before first study drug administration. For anti-PD-1 therapy no washout is required)

13. Therapy with agents known to prolong the QT interval and increase the risk for Torsades de Pointes, such as certain antibiotics (i.e. erythromycin, clarithromycin), antidepressants (i.e. doxepin, amitryptilin) or neuroleptics (i.e. haloperidol, clozapin)

14. Patients who have received a live vaccine within 28 days prior to anticipated first dose of study drug. Medical History related:

15. Subjects with a condition requiring systemic treatment with either corticosteroids (>10 mg daily prednisone equivalents) or other systemic immunosuppressive medications (e.g. methotrexate, azathioprine, mTOR inhibitors, Interferons, mycophenolate, anti-TNF agents, and other) within 14 days of study drug administration. Inhaled or topical steroids, and adrenal replacement doses <10 mg daily prednisone equivalents are permitted in the absence of active autoimmune disease. (Administration of steroids through a route known to result in minimal systemic exposure (topical, intranasal, intra-ocular, intra-articular or inhalation) are acceptable. Steroids as premedication for hypersensitivity reactions e.g. CT contrast are also acceptable)

16. Patients with any immunodeficiency disorder

17. Patients with any active, known or suspected autoimmune disease that might deteriorate when receiving an immunostimulatory agent as judged by the investigator

18. Patients with a marked baseline prolongation of QT/QTc interval, e.g., repeated demonstration of a QTc interval >450 msec (Grade 1 NCI-CTCAE); Long-QTSyndrome (QTcF is applicable)

19. Patients with any active gastrointestinal disorder that could interfere with the absorption of 4SC-202 (as per judgement of the investigator), such as ulcerative colitis, Crohn's disease, diabetic gastroparesis, or other syndromes characterized by malabsorption

20. Patients who are unable to take oral medication

21. Patients with a history of other malignancies unless having undergone definitive treatment more than 5 years prior to entry into the study and without evidence of recurrent malignant disease. Patients with basal cell carcinoma of the skin; superficial carcinoma of the bladder; carcinoma of the prostate or cervical intraepithelial neoplasia are eligible for the study if the disease has been clinically stable within the previous 3 years and no treatment is required

22. Patients with any other medical, psychiatric or social condition, which in the opinion of the investigator would preclude participation in the trial, pose an undue medical hazard, interfere with the conduct of the trial or interfere with interpretation of the trial results

23. Women who are pregnant or lactating or who are planning on becoming pregnant during the trial or for 90 days after completion of the trial

24. Patients with known HIV, acute or chronic active hepatitis B (defined as positive titers for HBsAg, anti-HBc-IgM or DNA) or Hepatitis C

25. Patients with an active systemic infection

26. Patients with major surgery within the last 4 weeks.

27. Patients with history or current evidence of clinically relevant allergies or hypersensitivity.

28. Patients with significant current cardiovascular disease including: a. Unstable angina pectoris within 6 months prior to screening b. Uncontrolled hypertension c. Congestive heart failure (New York Heart Association (NYHA) Class III or IV) related to primary cardiac disease d. Conditions requiring anti-arrhythmic therapy (patients with status post pace maker implantation can be included) e. Symptomatic ischemic or severe valvular heart disease, or a myocardial infarction within 6 months prior to the trial entry.

Tumor Assessments

Tumor assessments by physical examination and tumor imaging. CT scan or magnetic resonance imaging (MRI, if CT is not feasible due to safety or medical issue) of the chest, abdomen, pelvis and all other known sites of disease during screening, after end of Cycle 4 (within ±7 days of C5D1) and thereafter at the end of each 4th cycle, i.e. every 12 weeks (within ±7 days of Day 1 of Cycles 9, 13, 17 and so on). The last tumor assessment after 102 weeks (or 34 cycles, whatever is earlier) of completed treatment. The tumor assessment at EOT does not need to be repeated if a prior assessment has been performed within 6 weeks of EOT.

If pre-study images are available, then these can be used if the images are not older than 6 weeks relative to first treatment. In patients with known CNS/brain metastases, a MRI should be performed during screening. The same imaging technique as used at Screening should be used throughout the study, unless not possible due to safety (e.g. renal insufficiency) or if, after discussion with the investigator, the sponsor waives this requirement for individual assessments. Tumor response at each post-screening assessment is determined by the Investigator, based on irRECIST 1.131. Unscheduled imaging may be performed based on clinical indication and daily practice as based on Investigator's assessment. The Immune Related RECIST Criteria (irRECIST 1.1) is applicable for assessment of tumor response for the purposes of decision making for discontinuation of study therapy due to disease progression. The irRECIST response assessments are specifically described in the radiology manual. Tumor imaging assessments should continue until documented disease progression regardless of study treatment discontinuation or until subsequent treatment. Patients tolerating the study drug, showing signs of clinical benefit but showing progressive disease (irPD) at a tumor assessment may continue treatment until next tumor assessment for confirmation of irPD. Radiological confirmation of irCR is not mandatory.

1. A method for the treatment of cancer, comprising administering to a subject in need thereof an effective amount of an HDAC inhibitor of formula I in combination with at least one immune checkpoint modulator, 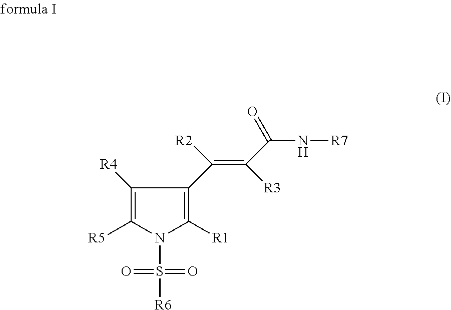
in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene,
either Q1 is substituted by R61 and/or R62, and is Aa1, Hh1, Ha1, Ha2, Ha3, Ha4 or Ah1, or Q1 is unsubstituted, and is Ha2, Ha3 or Ha4,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, —U-T3-N(R613)R614, -T4-Het3, or —V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups,
which are selected independently from a group consisting of phenyl and naphthyl, and which are linked together via a single bond,
Hh1 is a bisheteroaryl radical made up of two heteroaryl groups,
which are selected independently from a group consisting of monocyclic 5- or 6-membered heteroaryl radicals comprising one or two heteroatoms, each of which is selected from the group consisting of nitrogen, oxygen and sulfur, and
which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cyc1, in which Cyc1 is a ring system of formula Ia 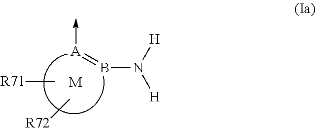
in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur.

2. The method according to claim 1, wherein the HDAC inhibitor is (E)-N-(2-aminophenyl)-3-(1-((4-(1-methyl-1H-pyrazol-4-yl)phenyl) sulfonyl)-1H-pyrrol-3-yl)acrylamide (also known as 4SC-202).

3. The method according to claim 2, wherein (E)-N-(2-aminophenyl)-3-(1-((4-(1-methyl-1H-pyrazol-4-yl)phenyl) sulfonyl)-1H-pyrrol-3-yl)acrylamide is administered in a dose of 150 to 250 mg/day, wherein the aforementioned daily doses are optionally administered in two portions, twice daily.

4. The method according to claim 1, wherein the at least one immune checkpoint modulator is selected from the group consisting of a) inhibitors of anti-inflammatory immune checkpoints including PD-1, CTLA-4, A2AR, B7-H3 (also known as CD276), B7-H4 (also known as VTCN1), BTLA, IDO, KIR, LAG3, TIM-3, VISTA (V-domain Ig suppressor of T cell activation) and their respective ligands including PD-L1, PD-L2, and galectin), and b) agonists of pro-inflammatory immune checkpoints including CD27, CD40, OX40, GITR, CD137, CD28, ICOS, and their respective ligands, including CD70 CD80, CD86, CD40L, CD137 ligand, OX40L, GITR ligand and ICOSL.

5. The method according to claim 1, wherein the at least one immune checkpoint modulator is selected from the group consisting of Ipilimumab, pembrolizumab, avelumab, nivolumab, durvalumab, tremelimumab, BCD-100 (Biocad), PDR-001 (Novartis), REGN-2810 (Regeneron), Camrelizumab (Shanghai Hengrui), SHR-1210 (Incyte), AGEN-2034 (Agenus), BGBA-317 (BeiGene), BMS-936559 (ViiV Healthcare), CX-072 (CytomX), CX-188 (CytomX), GNS-1480 (Genosco/Yuhan), IBI-308 (Eli Lilly/Innovent), JNJ-63723283 (J&J), JS-001 (Shanghai Junshi), MEDI-0680 (MedImmune), AMP-224 (MedImmune), BGB-A317 (BeiGene/Celgene), BI-754091 (Boehringer), CA-170 (Curis/Aurigene), CBT-501 (CBT Pharma), Genolimzunab (Genor), CBT-502 (CBT Pharma), FAZ-053 (Novartis), GLS-010 (Harbin/Wuxi/Arcus), AB122 (Harbin/Wuxi/Arcus), LY-3300054 (Eli Lilly), KN-035 (AlphaMab), M-7824 (Merck KGaA), MAG-012 (MacroGenics), MGD-013 (MacroGenics), PF-06801591 (Pfizer), SHR-1316 (Jiangsu Hengrui), TSR-042 (Tesaro), CS-1001 (CStone Pharma), HLX-10 (Shanghai Henlius), MCLA-145 (Merus/Incyte), AM-0001 (ARMO Bio), AVA-004 (Avacta), STI-al014 (Lee's Pharma/Sorrento), hAb-21 (Suzhou Stainwei), AK103 (Akeso Bio), AK104 (Akeso Bio), AK105 (Akeso Bio), AK106 (Akeso Bio), AK112 (Akeso Bio), BBI (Boston Biomedicals), BH-2922 (Beijing Hanmi), BH-2941 (Beijing Hanmi), BH 2950 (Beijing Hanmi), CA-327 (Curis/Aurigene), CBA-0710 (Sorrento), CK-301 (TG therapeutic), ENUM-244C8 (Enumeral), FS-118 (F-star Alpha/Merck KGaA), HTI-1316 (Hengrui Therapeutics), IKT-201 (Icell Kealex), IKT-202 (Icell Kealex), vaccinia virus expressing checkpoint inhibitor (Icell Kealex), JS-003 (Shanghai Junshi), JTX-4014 (Jounce/Celgene), KD033 (Kadmon/Jinghua Pharma), KY-1003 (Kymab), MCLA-134 (Merus), MSB-2311 (MABSPACE Bio), PRS-332 (Pieris/Servier), RXI-762 (Rxi Pharmaceuticals), SN-PD07 (Synovel), SN-PDL01 (Synovel), STI-A1110 (Sorrento/Servier), XmAb20717 (Xencor), AT16201 (AIMM), HLX-20 (Shanghai Henlius), IMM-1802 (ImmuneOnco Biopharma Shanghai), IMM-25 (ImmuneOnco Biopharma Shanghai), IMM-2502 (ImmuneOnco Biopharma Shanghai), IMM-2503 (ImmuneOnco Biopharma Shanghai), IMM-2504 (ImmuneOnco Biopharma Shanghai), CDX-1127 (Celldex Therapeutics NKTR-214 (Nektar Therapeutics), MEDI0562 (AstraZeneca), MEDI6469 (AstraZeneca), MEDI6383(AstraZeneca), MGA271 (MacroGenics), Lirilumab, and atezolizumab.

6. The method according to claim 5, wherein pembrolizumab is administered in a dose of 2 mg/kg, or in a dose of 200 mg, nivolumab is administered in a dose of 3 mg/kg, or in a dose of 240 mg or in a dose of 480 mg, ipilimumab is administered in a dose of 3 mg/kg or in a dose of 10 mg/kg, avelumab is administered in a dose of 10 mg/kg, atezolizumab is administered in a dose of 1200 mg, durvalumab is administered in a dose of 1500 mg, tremelimumab is administered in a dose of 1 mg/kg, in a dose of 75 mg.

7. The method according to claim 1, wherein the HDAC inhibitor is administered on days 1 to 14, or on days 1, 3, 5, 7, and 9, and the at least one immune checkpoint modulator is administered on day 1 in a 21-day treatment cycle, or wherein the HDAC inhibitor is administered on days 1 to 7, or on days 1, 3, and 5, and the at least one immune checkpoint modulator is administered on day 1 in a 14-day treatment cycle.

8. The method according to claim 1, wherein the treatment comprises a first treatment cycle wherein only the HDAC inhibitor is administered prior to administering the HDAC inhibitor and the immune checkpoint modulator.

9. The method according to claim 1, wherein the treatment comprises administering the HDAC inhibitor to the patient having said cancer in the non-fasted state.

10. The method according to claim 1, wherein said cancer is a solid tumor.

11. The method according to claim 1, wherein said cancer is selected from the group consisting of melanoma including ocular, uveal and skin melanoma, head and neck, renal, NSCLC, microsatellite-instable carcinoma including lynch syndrome including gastroesophageal and colorectal, urothelial carcinoma including bladder, merkel cell carcinoma, hodgkin lymphoma, gastric, oesophageal, non-hodgkin lymphoma, SCLC, sarkoma, mesothelioma, glioblastoma, microsatellite stable including gastroesophageal and colorectal, pancreas, HCC, prostata, basal cell carcinoma, CTCL, and squamous cell carcinoma.

12. The method according to claim 1, wherein said cancer is a refractory, non-responding or relapsed to immune checkpoint modulator therapy.

13. The method according to claim 1, wherein the patient having said cancer has received at least one prior systemic treatment against said cancer.

14. The method according to claim 1, wherein said cancer is resistant to immune checkpoint modulator therapy.
